SEQUENCE LISTING (74 sequences)

SEQ ID NO: 1 - DNA, 3381 bp, Homo sapiens
agaggaggaggaccggtcgtcgctgctgctgctgtcgccgcccgcggccaccgcctctcagacccagcagatcccaggcgggtccctggggtctgtgctgctgccagccgccaggttcgatgcccgggaggcggcggccgcggcggcggcggcgggggtgctgtacggaggggacgatgcccagggcatgatggcggcgatgctgtcccacgcctacggccccggcggttgtggggcggcggcggccgccctgaacggggagcaggcggccctgctccggagaaagagcgtcaacaccaccgagtgcgtcccggtgcccagctccgagcacgtcgccgagatcgtcggccgccagggttgtaaaattaaagcactgagagccaagacaaacacgtatatcaagactcctgttcgtggtgaagagcccatttttgttgtcactggaaggaaagaagatgttgccatggccaaaagagagatcctctcagctgcagagcacttctccatgattcgtgcatctcgaaacaaaaatgggcctgccctgggaggattatcatgtagtcctaatctgcccggtcaaaccaccgtccaagtcagggtcccttatcgtgtggtaggattagtggttggacccaaaggagcaactattaaaagaattcagcagcagacccacacctacatagtaactccgagcagagataaggaacctgtctttgaagtgacagggatgcctgaaaatgttgaccgagcacgggaagaaatagaaatgcatattgccatgcgtacaggaaactatatagagctcaatgaagagaatgatttccattacaatggtaccgatgtaagctttgaaggtggcactcttggctctgcgtggctctcctccaatcctgttcctcctagccgcgcaagaatgatatccaattatcgaaatgatagttccagttctctaggaagtggctctacagattcctactttggaagcaataggctggctgactttagtccaacaagcccatttagcacaggaaacttctggtttggagatacactaccatctgtaggctcagaagacctagcagttgactctcctgcctttgactctttaccaacatctgctcaaactatctggactccatttgaaccagttaacccactctctggctttgggagtgatccttctggtaacatgaagactcagcgcagaggaagtcagccatctactcctcgtctgtctcctacatttcctgagagcatagaacatccacttgctcggagggttaggagcgacccacctagtacaggcaaccatgttggccttccaatatatatccctgctttttctaatggtaccaatagttactcctcttccaatggtggttccacctctagctcacctccagaatcaagacgaaagcatgactgtgtgatttgctttgagaatgaggttattgctgccctagttccatgtggccacaacctcttctgcatggaatgtgccaacaagatctgtgaaaagagaacgccatcatgtccagtttgccagacagctgttactcaggcaatccaaattcactcttaactatatatatatacataaatactatatctctatatggactcgtaaaggcatgggtataatggtaccccccagtaaacttcctaatgatttcttatgactgttatcaggctttattgggattaggctaaagttgttagtaaacttataaaaggctgctatggtaacactaaacctaagtggtctcttgtctattagtttggtttgaattattagtactatcctgtagacccagagacatagtttatataagaattgctaaagctgaagttcaacttggctgagtgaagataatcataggttgtgtgagcctatgaaaaagtgtatacgtctaagatttcaaaacaatgggtcccaaagcctaaccactttaagagtttatggagggtacttggcattacagacgattcatacacttccagtgctgccttctttacactgccagttttgacaaaacaggtttgttttttattttacaacaacatatgcctaattctgcaggattgcaagtaactttttaatgcattgtgattacttattggtaatgatagggctgatggcagtttactagatcactggttataatttgggacaaaaactgctacatcaactttcatctcgcccagagtgctcaaggctggtatgatcagtggatcaggaatgcaattgtgaattcctgcccattgcctctcttggtgaatgtggaaatggccacctgggttttcccatatcaggaagggctttgggatggcacctatattggctgataattgaggatgcaaacattccattcattagtgtgatcgagctgttaatttttagactatagatcaaaatgtgaaacattttatgttcaatccatatttgtcttgcacattataaatatatttttattttttagtaatttaggggagggaggagggagaaagggataatgatgcccttggcataattcacaaaaacagctgtgacaacctccaatcagtttacttcatttcaaaactatttccaatcacaaggaaagatttatttaaaatatactcgtacatttcacctgtggatgtctataacttcatcctcagtatgttcccaaatctgtgctggcattgaaaggacaaaacattatactagtgggtttttctactaattattttttgaagcattattttcccaacacaaaagagcttttttctcggtataatgaaaattgaaatcctatgtgtattcaatagtaaatagacaaattttattttttatttccacttgaagagttacatttcgtataaaagtttacaaataacggtttttattttgattttttcagtataaaaaaagttgccttgatggcatattatgatgtaatgctaattgcttgtaggatagtaaatggtcagtattgaaacctaatctctagctgccgtcttgtagatatgaacgaatgttcaccaagcatgtattttgtattttgttgcattgtacactgcaactaataagccaaggaatcgacatatattaggtgcgtgtactgtttctaaaaaccacaaactaagaatgataaattatcaatatagtttagtatttgctaattttactacactcttttgttatgtatatgtagggaagtcatagggattataaattcaatttgagtaaaatttaaaaccatatattttatgataaagggcctttaacttaagatggccaaagcactgatattatatatttgctgtaaagagaattataagagttttatttttctgatattaaaagttacttaataaagacttgtttccattaacttg

SEQ ID NO: 2 - protein, 464 aa, Homo sapiens
MMAAMLSHAYGPGGCGAAAAALNGEQAALLRRKSVNTTECVPVPSSEHVAEIVGRQGCKIKALRAKTNTYIKTPVRGEEPIFVVTGRKEDVAMAKREILSAAEHFSMIRASRNKNGPALGGLSCSPNLPGQTTVQVRVPYRVVGLVVGPKGATIKRIQQQTHTYIVTPSRDKEPVFEVTGMPENVDRAREEIEMHIAMRTGNYIELNEENDFHYNGTDVSFEGGTLGSAWLSSNPVPPSRARMISNYRNDSSSSLGSGSTDSYFGSNRLADFSPTSPFSTGNFWFGDTLPSVGSEDLAVDSPAFDSLPTSAQTIWTPFEPVNPLSGFGSDPSGNMKTQRRGSQPSTPRLSPTFPESIEHPLARRVRSDPPSTGNHVGLPIYIPAFSNGTNSYSSSNGGSTSSSPPESRRKHDCVICFENEVIAALVPCGHNLFCMECANKICEKRTPSCPVCQTAVTQAIQIHS

SEQ ID NO: 3 - DNA, 3022 bp, Homo sapiens
caagacaaacacgtatatcaagactcctgttcgtggtgaagagcccatttttgttgtcactggaaggaaagaagatgttgccatggccaaaagagagatcctctcagctgcagagcacttctccatgattcgtgcatctcgaaacaaaaatgggcctgccctgggaggattatcatgtagtcctaatctgcccggtcaaaccaccgtccaagtcagggtcccttatcgtgtggtaggattagtggttggacccaaaggagcaactattaaaagaattcagcagcagacccacacctacatagtaactccgagcagagataaggaacctgtctttgaagtgacagggatgcctgaaaatgttgaccgagcacgggaagaaatagaaatgcatattgccatgcgtacaggaaactatatagagctcaatgaagagaatgatttccattacaatggtaccgatgtaagctttgaaggtggcactcttggctctgcgtggctctcctccaatcctgttcctcctagccgcgcaagaatgatatccaattatcgaaatgatagttccagttctctaggaagtggctctacagattcctactttggaagcaataggctggctgactttagtccaacaagcccatttagcacaggaaacttctggtttggagatacactaccatctgtaggctcagaagacctagcagttgactctcctgcctttgactctttaccaacgtctgctcaaactatctggactccatttgaaccagttaacccactctctggctttgggagtgatccttctggtaacatgaagactcagcgcagaggaagtcagccatctactcctcgtctgtctcctacatttcctgagagcatagaacatccacttgctcggagggttaggagcgacccacctagtacaggcaaccatgttggccttccaatatatatccctgctttttctaatggtaccaatagttactcctcttccaatggtggttccacctctagctcacctccagaatcaagacgaaagcacgactgtgtgatttgctttgagaatgaggttattgctgccctagttccatgtggccacaacctcttctgcatggaatgtgccaacaagatctgtgaaaagagaacgccatcatgtccagtttgccagacagctgttactcaggcaatccaaattcactcttaactatatatatatacataaatactatatctctatatggactcgtaaaggcatgggtataatggtaccccccagtaaacttcctaatgatttcttatgactgttatcaggctttattgggattaggctaaagttgttagtaaacttataaaaggctgctatggtaacactaaacctaagtggtctcttgtctattagtttggtttgaattattagtactatcctgtagacccagagacatagtttatataagaattgctaaagctgaagttcaacttggctgagtgaagataatcataggttgtgtgagcctatgaaaaagtgtatacgtctaagatttcaaaacaatgggtcccaaagcctaaccactttaagagtttatggagggtacttggcattacagacgattcatacacttccagtgctgccttctttacactgccagttttgacaaaacaggtttgttttttattttacaacaacatatgcctaattctgcaggattgcaagtaactttttaatgcattgtgattacttattggtaatgatagggctgatggcagtttactagatcactggttataatttgggacaaaaactgctacatcaactttcatctcgcccagagtgctcaaggctggtatgatcagtggatcaggaatgcaattgtgaattcctgcccattgcctctcttggtgaatgtggaaatggccacctgggttttcccatatcaggaagggctttgggatggcacctatattggctgataattgaggatgcaaacattccattcattagtgtgatcgagctgttaatttttagactatagatcaaaatgtgaaacattttatgttcaatccatatttgtcttgcacattataaatatatttttattttttagtaatttaggggagggaggagggagaaagggataatgatgcccttggcataattcacaaaagcagctgtgacaacctccaatcagtttacttcatttcaaaactatttccaatcacaaggaaagatttatttaaaatatactcgtacatttcacctgtggatgtctataacttcatcctcagtatgttcccaaatctgtgctggcattgaaaggacaaaacattatactagtgggtttttctactaattattttttgaagcattattttcccaacacaaaagagcttttttctcggtataatgaaaattgaaatcctatgtgtattcaatagtaaatagacaaattttattttttatttccacttgaagagttacatttcgtataaaagtttacaaataacggtttttattttgattttttcagtataaaaaaagttgccttgatggcatattatgatgtaatgctaattgcttgtaggatagtaaatggtcagtattgaaacctaatctctagctgccgtcttgtagatatgaacgaatgttcaccaagcatgtattttgtattttgttgcattgtacactgcaactaataagccaaggaatcgacatatattaggtgcgtgtactgtttctaaaaaccacaaactaagaatgataaattatcaatatagtttagtatttgctaattttactacactcttttgttatgtatatgtagggaagtcatagggattataaattcaatttgagtaaaatttaaaaccatatattttatgataaagggcctttaacttaagatggccaaagcactgatattatatatttgctgtaaagagaattataagagttttatttttctgatattaaaagttacttgataaagacttgtttccattaacttgaaaaaaaaaaaaaaaaaaaaaa

SEQ ID NO: 4 - protein, 372 aa, Homo sapiens
MAKREILSAAEHFSMIRASRNKNGPALGGLSCSPNLPGQTTVQVRVPYRVVGLVVGPKGATIKRIQQQTHTYIVTPSRDKEPVFEVTGMPENVDRAREEIEMHIAMRTGNYIELNEENDFHYNGTDVSFEGGTLGSAWLSSNPVPPSRARMISNYRNDSSSSLGSGSTDSYFGSNRLADFSPTSPFSTGNFWFGDTLPSVGSEDLAVDSPAFDSLPTSAQTIWTPFEPVNPLSGFGSDPSGNMKTQRRGSQPSTPRLSPTFPESIEHPLARRVRSDPPSTGNHVGLPIYIPAFSNGTNSYSSSNGGSTSSSPPESRRKHDCVICFENEVIAALVPCGHNLFCMECANKICEKRTPSCPVCQTAVTQAIQIHS

SEQ ID NO: 5 - DNA, 1513 bp, Homo sapiens
cctgaacggggagcaggcggccctgctccggagaaagagcgtcaacaccaccgagtgcgtcccggtgcccagctccgagcacgtcgccgagatcgtcggccgccagggttgtaaaattaaagcactgagagccaagacaaacacgtatatcaagactcctgttcgtggtgaagagcccatttttgttgtcactggaaggaaagaagatgttgccatggccaaaagagagatcctctcagctgcagagcacttctccatgattcgtgcatctcgaaacaaaaatgggcctgccctgggaggattatcatgtagtcctaatctgcccggtcaaaccaccgtccaagtcagggtcccttatcgtgtggtaggattagtggttggacccaaaggagcaactattaaaagaattcagcagcagacccacacctacatagtaactccgagcagagataaggaacctgtctttgaagtgacagggatgcctgaaaatgttgaccgagcacgggaagaaatagaaatgcatattgccatgcgtacaggaaactatatagagctcaatgaagagaatgatttccattacaatggtaccgatgtaagctttgaaggtggcactcttggctctgcgtggctctcctccaatcctgttcctcctagccgcgcaagaatgatatccaattatcgaaatgatagttccagttctctaggaagtggctctacagattcctactttggaagcaataggctggctgactttagtccaacaagcccatttagcacaggaaacttctggtttggagatacactaccatctgtaggctcagaagacctagcagttgactctcctgcctttgactctttaccaacatctgctcaaactatctggactccatttgaaccagttaacccactctctggctttgggagtgatccttctggtaacatgaagactcagcgcagaggaagtcagccatctactcctcgtctgtctcctacatttcctgagagcatagaacatccacttgctcggagggttaggagcgacccacctagtacaggcaaccatgttggccttccaatatatatccctgctttttctaatggtaccaatagttactcctcttccaatggtggttccacctctagctcacctccagaatcaagacgaaagcacgactgtgtgatttgctttgagaatgaggttattgctgccctagttccatgtggccacaacctcttctgcatggaatgtgccaacaagatctgtgaaaagagaacgccatcatgtccagtttgccagacagctgttactcaggcaatccaaattcactcttaactatatatatatacataaatactatatctctatatggactcgtaaaggcatgggtataatggtaccccccagtaaacttcctaatgatttcttatgactgttatcaggctttattgggattaggctaaagttgttagtaaacttataaaaggctgctatggtaacactaaaaaaaaaaaa

SEQ ID NO: 6 - protein, 372 aa, Homo sapiens; identical to SEQ ID NO: 4

SEQ ID NO: 7 - DNA, 2286 bp, Homo sapiens
aactatgtggactccattttgaccagttaacccattctcgtggctttgggagtatccttctggtaacatgaagactcagcgcagaggaagtcagccatctactcctcgtctgtctcctacatttcctgagagcatagaacatccacttgctcggagggttaggagcgacccacctagtacaggcaaccatgttggccttccaatatatatccctgctttttctaatggtaccaatagttactcctcttccaatggtggttccacctctagctcacctccagaatcaagacgaaagcacgactgtgtgatttgctttgagaatgaggttattgctgccctagttccatgtggccacaacctcttctgcatggaatgtgccaacaagatctgtgaaaagagaacgccatcatgtccagtttgccagacagctgttactcaggcaatccaaattcactcttaactatatatatatacataaatactatatctctatatggactcgtaaaggcatgggtataatggtaccccccagtaaacttcctaatgatttcttatgactgttatcaggctttattgggattaggctaaagttgttagtaaacttataaaaggctgctatggtaacactaaacctaagtggtctcttgtctattagtttggtttgaattattagtactatcctgtagacccagagacatagtttatataagaattgctaaagctgaagttcaacttggctgagtgaagataatcataggttgtgtgagcctatgaaaaagtgtatacgtctaagatttcaaaacaatgggtcccaaagcctaaccactttaagagtttatggagggtacttggcattacagacgattcatacacttccagtgctgccttctttacactgccagttttgacaaaacaggtttgttttttattttacaacaacatatgcctaattctgcaggattgcaagtaactttttaatgcattgtgattacttattggtaatgatagggctgatggcagtttactagatcactggttataatttgggacaaaaactgtacatcaactttcatctcgcccagagtgtcaaggctggtatgatcagtggatcaggaatgcaattgtgaattcctgcccattgcctctcttggtgaatgtggaaatggccacctgggttttcccatatcaggaagggctttgggatagcacctatattggctgataattgaggatgcaaacattccatcattagtgtgatcgagctgttaatttttagactatagatcaaaatgtgaaacattttatgttcaatccatatttgtcttgcacattataaatatatttttattttttagtaatttaggggagggaggagggagaaagggataatgatgcccttggcataatttcacaaaagcagctgtgacaacctccaatcagtttacttcatttcaaaactatttccaatcacaaggaaagatttatttaaaatatactcgtacatttcacctgtggatgtctataacttcatcctcagtatgttcccaaatctgtgctggcattgaaaggacaaaacattatactagtgggtttttctactaattattttttgaagcattattttcccaacacaaaagagcttttttctcggtataatgaaaattgaaatcctatgtgtattcaatagtaaatagacaaattttattttttatttccacttgaagagttacatttcgtataaaagtttacaaataacggtttttattttgattttttcagtataaaaaaagttgccttgatggcatattatgatgtaatgctaattgcttgtaggatagtaaatggtcagtattgaaacctaatctctagctgccgtcttgtagatatgaacgaatgttcaccaagcatgtattttgtattttgttgcattgtacactgcaactaataagccaaggaatcgacatatattaggtgcgtgtactgtttctaaaaaccacaaactaagaatgataaattatcaatatagtttagtatttgctaattttactacactcttttgttatgtatatgtagggaagtcatagggattataaattcaatttgagtaaaatttaaaaccatatattttatgataaagggcctttaacttaagatggccaaagcactgatattatatatttgctgtaaagagaattataagagttttatttttctgatattaaaagttacttaataaagacttgtttccattaacttgaaaaaaaaaaaaaaaaaaaaaaaaaaaaa

SEQ ID NO: 8 - protein, 130 aa, Homo sapiens
MKTQRRGSQPSTPRLSPTFPESIEHPLARRVRSDPPSTGNHVGLPIYIPAFSNGTNSYSSSNGGSTSSSPPESRRKHDCVICFENEVIAALVPCGHNLFCMECANKICEKRTPSCPVCQTAVTQAIQIHS

SEQ ID NO: 9 - DNA, 2286 bp, Homo sapiens; identical to SEQ ID NO: 7

SEQ ID NO: 10 - protein, 130 aa, Homo sapiens; identical to SEQ ID NO: 8

SEQ ID NO: 11 - DNA, 2446 bp, Homo sapiens
taggaagtggctctacagattcctactttggaagcaataggctggctgactttagtccaacaagcccatttagcacaggaaacttctggtttggagatacactaccatctgtaggctcagaagacctagcagttgactctcctgcctttgactctttaccaacatctgctcaaactatctggactccatttgaaccagttaacccactctctggctttgggagtgatccttctggtaacatgaagactcagcgcagaggaagtcagccatctactcctcgtctgtctcctacatttcctgagagcatagaacatccacttgctcggagggttaggagcgacccacctagtacaggcaaccatgttggccttccaatatatatccctgctttttctaatggtaccaatagttactcctcttccaatggtggttccacctctagctcacctccagaatcaagacgaaagcacgactgtgtgatttgctttgagaatgaggttattgctgccctagttccatgtggccacaacctcttctgcatggaatgtgccaacaagatctgtgaaaagagaacgccatcatgtccagtttgccagacagctgttactcaggcaatccaaattcactcttaactatatatatatacataaatactatatctctatatggactcgtaaaggcatgggtataatggtaccccccagtaaacttcctaatgatttcttatgactgttatcaggctttattgggattaggctaaagttgttagtaaacttataaaaggctgctatggtaacactaaacctaagtggtctcttgtctattagtttggtttgaattattagtactatcctgtagacccagagacatagtttatataagaattgctaaagctgaagttcaacttggctgagtgaagataatcataggttgtgtgagcctatgaaaaagtgtatacgtctaagatttcaaaacaatgggtcccaaagcctaaccactttaagagtttatggagggtacttggcattacagacgattcatacacttccagtgctgccttctttacactgccagttttgacaaaacaggtttgttttttattttacaacaacatatgcctaattctgcaggattgcaagtaactttttaatgcattgtgattacttattggtaatgatagggctgatggcagtttactagatcactggttataatttgggacaaaaactgctacatcaactttcatctcgcccagagtgctcaaggctggtatgatcagtggatcaggaatgcaattgtgaattcctgcccattgcctctcttggtgaatgtggaaatggccacctgggttttcccatatcaggaagggctttgggatggcacctatattggctgataattgaggatgcaaacattccattcattagtgtgatcgagctgttaatttttagactatagatcaaaatgtgaaacattttatgttcaatccatatttgtcttgcacattataaatatatttttattttttagtaatttaggggagggaggagggagaaagggataatgatgcccttggcataattcacaaaagcagctgtgacaacctccaatcagtttacttcatttcaaaactatttccaatcacaaggaaagatttatttaaaatatactcgtacatttcacctgtggatgtctataacttcatcctcagtatgttcccaaatctgtgctggcattgaaaggacaaaacattatactagtgggtttttctactaattattttttgaagcattattttcccaacacaaaagagcttttttctcggtataatgaaaattgaaatcctatgtgtattcaatagtaaatagacaaattttattttttatttccacttgaagagttacatttcgtataaaagtttacaaataacggtttttattttgattttttcagtataaaaaaagttgccttgatggcatattatgatgtaatgctaattgcttgtaggatagtaaatggtcagtattgaaacctaatctctagctgccgtcttgtagatatgaacgaatgttcaccaagcatgtattttgtattttgttgcattgtacactgcaactaataagccaaggaatcgacatatattaggtgcgtgtactgtttctaaaaaccacaaactaagaatgataaattatcaatatagtttagtatttgctaattttactacactcttttgttatgtatatgtagggaagtcatagggattataaattcaatttgagtaaaatttaaaaccatatattttatgataaagggcctttaacttaagatggccaaagcactgatattatatatttgctgtaaagagaattataagagttttatttttctgatattaaaagttacttaataaagacttgtttccattaacttgaaaaaaaaaaaaaaa

SEQ ID NO: 12 - protein, 130 aa, Homo sapiens; identical to SEQ ID NO: 8

SEQ ID NO: 13 - DNA, 3870 bp, Homo sapiens
agcggtacggaggggacgatgcccagggcatgatggcggcgatgctgtcccacgcctacggccccggcggttgtggggcggcggcggccgccctgaacggggagcaggcggccctgctccggagaaagagcgtcaacaccaccgagtgcgtcccggtgcccagctccgagcacgtcgccgagatcgtcggccgccagggtgagtgcaggggtggggaggtccagctgggcatggccccgggcgtgggtggggagacgagagaaaggagcgtgggacggaaaaggcgaacccggcgaggcccccaagtccaccgcgaaggggtctgggaggagccgagggtcgaccagatgtgaagatgggtcaccttgcgaaaacccacgcttttccgcccgtcttgcctggtaaaaagtgtgaagtttctcgggcgacccagtccggtggtgttcacgttacttgggctgtattcgtggaacaccaggctggcgtggcaccagaaacctcagtgaggtggtaaaaggtcgggctcgaggagttgtgggttcgctgttgggtacggaaagcttcgagattgcaagtttgcgccgccgagtgtcttctaaaagcgttcagcctcccttacaagaacttgttcagatggagaaaagaacagaaatggaggcctgtagacagagctcactggggagggaaggggtgttcaaatggtcggtatcaccgccatcccctgcccctccgctgttgagaaacttagctttggaaaagcagcccaactcactgccgcctatcttaactgtatttctctcggtacgtttcaactctcggaaagacacactgacacactggctgtcttttaaaactgttcagtgtgcaggatgaagaaaaaatggacactaaggtaattaagaaacttcatcggtttatacagttttcagttaattctcctcttcacttgccagcacaccagtactgcagacagactcctcccacattgttcagacaaaggacctgtgtctcagattccaggagtagatttgtgaaggtgggggaaggggatgcccggggtacctctgtatccgacatttttagggaaggtcctaagttctttatggtagcatttcccctttatgaaaggggcagatgttacctgctgccaccgcatgacaatgtggtttagcctggttgttgcatgcctctttactgttaggttgagagcattgttggggtcaaataaagagagatgatggcgctgccgcttaaatgatctcatctttcccaggtggtctaaagtacacataaataagagctctgtcgaaatggtggtttcacatatcagacttggcttatttctttcctatctgtaggattttaataaaaactcatctgaaatctggataaaggagatagcttagttcagactgcctgatgattggcacttacagacactgttcaaagctggacctttcaattgaaaggtagcttatgcttgtcgttcttctggttaatgagtttcagattcctggattgtttggttttggcgatacttagagtaataatccaggagaaaacttttgtttacatgggataaaattacaacatctcaatttggatcagaatgagttgaccattcatgtaagaacactgaggaatcgctatgtaccaagcttgtattttgccaggttttgagtttgtggtattgagaatgtgaagttaattcagactttctgcccttaagaagataacagtctaataaacagtagaatggcactaactatagtgtatttagtatgacaaagtctcaggtaagagaacatctatggagagggccttggaaaatgggtaggaaggagtctaacaaatggagattgaaaaactgtggaggaacaatgtgaggaggggtgtggaggactagcctaggtataattgccaagtagtgttgttttgatacttgaggtctgtaaaggggagtggtggaaaagggggaaggcaggcgttcacaccagaatgtgaagaagcctgaatgccatatgaagatgccacttctcaggacaagttgcttccgtgtgcttatggtaacagtctagggtaacaaaggaagaaaacatttacgtgccagttttactgaaggacaagataacatattttatagatttagtttaatacaaactttagtccttcattatagtagacaccccttaaagcactgctaaatggcccttacttgtgtatgttgttaatctttatagaaatcagttttttaaaagtaaagtgaattccaagtttttattttgtaccagctattacgtgataggaaatgagataaagccttagtttttcttttgagggctgtgtttatttggttaataaggaaatgttgaatttaattatgtcttagatcatggagacaaattgctggctcttccatttaaaccttgtattttcccagagttgacaatactgttaaaagtatgtcatcatcctctatcttaaaagcagaaaatctggtccttttgctgttctgaaccatacaacttgtggatcattgacccctttgccacccccatgtcccagcagcatagtaataaacctgtaaagtgtttgctcactcgtatggggaacgaagtgacttctaatgttcacattcttttacagtattatatttccccacgggagagaatctgtcctaagtaagtaaaacagggcaaatcatttcattttcttgtttgctttgaaggagctaaactttcatccttaagattaaattatccatttctgccattttatgtattacctgaagccataattttctatttgacatctcttaattgattttggaaataattatgtcacagatggagacatttatgacttcagctctgaagcaaagagcaggacaaatggaattctgagtcacatagactagcaactcatgtagatggcctctgagttccagtgtttttctaaaacacaaactaccgcttttgatactgatgaaagttggcagagtgtttgacctcgaatttatactccccagtagcatatggtcatttgtaaatgttaaattggccttgttttttaaagcattgcatctgttgtgaaacaacaatactgtaaaataaagccaggaactgaagaggcttgttgatataatcaaagaagttttagaatatggacatcaaaaataaacctgacaacagattttaagacctaattttataaacttagaacgtctctgtttttaagtatagtacctgcaagacatattaacagtgattaacaggtctaattttagtcagcaggttttgttccaatttgtacagtcatttatatccatttagactaaaaagtgaattaaaatagaactgtatacccattcttaatacattataaattaatactaaatgtcataaattcagttgtttagagacttgtaacccaagattttgtattactattaggataaatgtgtatctttcattagactttaattaatggtctatgtgtgttctctagccacatagtctagaaaagtcgtctctagcttctttttattgtacaccctgtccaaaaaaaatgtgcatactcccaatatctggatatttatgtatgaatagtaagtgtgcttaatatattacacattataaaacaaaattctttattaacaattaatactttacactgtttcaatttgtcttcccaatttcttagggtggaaccacccttcaagctctaacacaactgaaatagggtttatacgtctgtgaccaggagaaaaagagaaagcttggtcttgctgtacataaattttctcttcaaggagaagtacatatttcatgagaataaatagtttcagaatgatgcaaaaaaaaaaaaaaa

SEQ ID NO: 14 - protein, 107 aa, Homo sapiens
MMAAMLSHAYGPGGCGAAAAALNGEQAALLRRKSVNTTECVPVPSSEHVAEIVGRQGECRGGEVQLGMAPGVGGETRERSVGRKRRTRRGPQVHREGVWEEPRVDQM

SEQ ID NO: 15 - DNA, 654 bp, Homo sapiens
agcggatcacaactctgccactgccatcagcaggaacaacccagctttcaccttccactgcttgccatcccaaaggttgtaaaattaaagcactgagagccaagacaaacacgtatatcaagactcctgttcgtggtgaagagcccatttttgttgtcactggaaggaaagaagatgttgccatggccaaaagagagatcctctcagctgcagagcacttctccatgattcgtgcatctcgaaacaaaaatgggcctgccctgggaggattatcatgtagtcctaatctgcccggtcaaaccaccgtccaagtcagggtcccttatcgtgtggtaggattagtggttggacccaaaggagcaactattaaaagaattcagcagcagacccacacctacatagtaactccgagcagagataaggaacctgtctttgaagtgacagggatgcctgaaaatgttgaccgagcacgggaagaaatagaaatgcatattgccatgcgtacaggaaactatatagagctcaatgaagggaatgatttccattacaatggtaccgatgtaagctttgaaggtggcactcttggctctgcgtggctctcctccaatcctgttcctcctagccgcgcaagaatgatatccaattatcgaaatg

SEQ ID NO: 16 - protein, 157 aa, Homo sapiens
MAKREILSAAEHFSMIRASRNKNGPALGGLSCSPNLPGQTTVQVRVPYRVVGLVVGPKGATIKRIQQQTHTYIVTPSRDKEPVFEVTGMPENVDRAREEIEMHIAMRTGNYIELNEGNDFHYNGTDVSFEGGTLGSAWLSSNPVPPSRARMISNYRN

SEQ ID NO: 17 - DNA, 653 bp, Homo sapiens
agcggatcacaactctgccactgccatcagcaggaacaacccagctttcaccttccactgcttgccatcccaaaggttgtaaaattaaagcactgagagccaagacaaacacgtatatcaagactcctgttcgtggtgaagagcccatttttgttgtcactggaaggaaagaagatgttgccatggtcaaaagagagatcctctcagctgcagagcacttctccatgattcgtgcatctcgaaacaaaaatgggcctgccctgggaggattatcatgtagtcctaatctgcccggtcaaaccaccgtccaagtcagggtcccttatcgtgtggtaggattagtggttggacccaaaggagcaactattaagaagaattcagcagcagaccacacctacatagtaactccgagcagagataaggaacctgtctttgaagtgacagggatgcctgaaaatgttgaccgagcacgggaagaaatagaaatgcatattgccatgcgtacaggaaactatatagagctcaatgaagggaatgatttccattacaatggtaccgatgtaagctttgaaggtggcactcttggctctgcgtggtctcctccaatcctgttcctcctagccgcgcaagaatgatatccaattatcgaaatt

SEQ ID NO: 18 - protein, 150 aa, Homo sapiens
MVKREILSAAEHFSMIRASRNKNGPALGGLSCSPNLPGQTTVQVRVPYRVVGLVVGPKGATIKKNSAADHTYIVTPSRDKEPVFEVTGMPENVDRAREEIEMHIAMRTGNYIELNEGNDFHYNGTDVSFEGGTLGSAWSPPILFLLAAQE

SEQ ID NO: 19 - DNA, 801 bp, Homo sapiens
ggaacaacccagctttcaccttccactgcttgccatcccaaaggttgtaaaattaaagcactgagagccaagacaaacacgtatatcaagactcctgttcgtggtgaagagcccatttttgttgtcactggaaggaaagaagatgttgccatggccaaaagagagatcctctcagctgcagagcacttctccatgattcgtgcatctcgaaacaaaaatgggcctgccctgggaggattatcatgtagtcctaatctgcccggtcaaaccaccgtccaagtcagggtcccttatcgtgtggtaggattagtggttggacccaaaggagcaactattaaaagaattcagcagcagacccacacctacatagtaactccgagcagagataaggaacctgtctttgaagtgacagggatgcctgaaaatgttgaccgagcacgggaagaaatagaaatgcatattgccatgcgtacaggaaactatatagagctcaatgaagagaatgatttccattacaatggtaccgatgtaagctttgaaggtggcactcttggctctgcgtggctctcctccaatcctgttcctcctagccgcgcaagaatgatatccaattatcgaaatgatagttccagttctctaggaaagtggctctacagattcctactttggaagcaataggctggctgactttagtccaacaagcccatttagcacaggaaacttctgggtatggagatacactaccatctgtaggctcagaagacctagcagttgactctcctggctttgactctttaccaa

SEQ ID NO: 20 - protein, 175 aa, Homo sapiens
MAKREILSAAEHFSMIRASRNKNGPALGGLSCSPNLPGQTTVQVRVPYRVVGLVVGPKGATIKRIQQQTHTYIVTPSRDKEPVFEVTGMPENVDRAREEIEMHIAMRTGNYIELNEENDFHYNGTDVSFEGGTLGSAWLSSNPVPPSRARMISNYRNDSSSSLGKWLYRFLLWKQ

SEQ ID NO: 21 - DNA, 21 bp, Homo sapiens
aaccaccgtccaagtcagggt

SEQ ID NO: 22 - DNA, 3707 bp, Homo sapiens
atgcccagcggcagctccgcggccctggccctggcggcggccccggcccccctgccgcagccgcccccgccgccgccgccgccaccgccgcctctgccgccgccctcgggcggcccggagctcgagggggacgggctcctgctgagggagcgcttggccgcgctaggcctcgacgaccccagcccggcggagcccggcgccccggcgcttcgggccccggcagcggcggcgcagggccaggcccggcgggcggcggagctgtctccagaggagcgggctccgcccggccggcccggggccccggaggcggccgagctggagctggaagaggaggaggaccggtcgtcgctgctgctgctgtcgccgcccgcggccaccgcctctcagacccagcagatcccaggcgggtccctggggtctgtgctgctgccagccgccaggttcgatgcccgggaggcggcggccgcggcggcggcggcgggggtgctgtacggaggggacgatgcccagggcatgatggcggcgatgctgtcccacgcctacggccccggcggttgtggggcggcggcggccgccctgaacggggagcaggcggccctgctccggagaaagagcgtcaacaccaccgagtgcgtcccggtgcccagctccgagcacgtcgccgagatcgtcggccgccagggttgtaaaattaaagcactgagagccaagacaaacacgtatatcaagactcctgttcgtggtgaagagcccatttttgttgtcactggaaggaaagaagatgttgccatggccaaaagagagatcctctcagctgcagagcacttctccatgattcgtgcatctcgaaacaaaaatgggcctgccctgggaggattatcatgtagtcctaatctgcccggtcaaaccaccgtccaagtcagggtcccttatcgtgtggtaggattagtggttggacccaaaggagcaactattaaaagaattcagcagcagacccacacctacatagtaactccgagcagagataaggaacctgtctttgaagtgacagggatgcctgaaaatgttgaccgagcacgggaagaaatagaaatgcatattgccatgcgtacaggaaactatatagagctcaatgaagagaatgatttccattacaatggtaccgatgtaagctttgaaggtggcactcttggctctgcgtggctctcctccaatcctgttcctcctagccgcgcaagaatgatatccaattatcgaaatgatagttccagttctctaggaagtggctctacagattcctactttggaagcaataggctggctgactttagtccaacaagcccatttagcacaggaaacttctggtttggagatacactaccatctgtaggctcagaagacctagcagttgactctcctgcctttgactctttaccaacatctgctcaaactatctggactccatttgaaccagttaacccactctctggctttgggagtgatccttctggtaacatgaagactcagcgcagaggaagtcagccatctactcctcgtctgtctcctacatttcctgagagcatagaacatccacttgctcggagggttaggagcgacccacctagtacaggcaaccatgttggccttccaatatatatccctgctttttctaatggtaccaatagttactcctcttccaatggtggttccacctctagctcacctccagaatcaagacgaaagcatgactgtgtgatttgctttgagaatgaggttattgctgccctagttccatgtggccacaacctcttctgcatggaatgtgccaacaagatctgtgaaaagagaacgccatcatgtccagtttgccagacagctgttactcaggcaatccaaattcactcttaactatatatatatacataaatactatatctctatatggactcgtaaaggcatgggtataatggtaccccccagtaaacttcctaatgatttcttatgactgttatcaggctttattgggattaggctaaagttgttagtaaacttataaaaggctgctatggtaacactaaacctaagtggtctcttgtctattagtttggtttgaattattagtactatcctgtagacccagagacatagtttatataagaattgctaaagctgaagttcaacttggctgagtgaagataatcataggttgtgtgagcctatgaaaaagtgtatacgtctaagatttcaaaacaatgggtcccaaagcctaaccactttaagagtttatggagggtacttggcattacagacgattcatacacttccagtgctgccttctttacactgccagttttgacaaaacaggtttgttttttattttacaacaacatatgcctaattctgcaggattgcaagtaactttttaatgcattgtgattacttattggtaatgatagggctgatggcagtttactagatcactggttataatttgggacaaaaactgctacatcaactttcatctcgcccagagtgctcaaggctggtatgatcagtggatcaggaatgcaattgtgaattcctgcccattgcctctcttggtgaatgtggaaatggccacctgggttttcccatatcaggaagggctttgggatggcacctatattggctgataattgaggatgcaaacattccattcattagtgtgatcgagctgttaatttttagactatagatcaaaatgtgaaacattttatgttcaatccatatttgtcttgcacattataaatatatttttattttttagtaatttaggggagggaggagggagaaagggataatgatgcccttggcataattcacaaaaacagctgtgacaacctccaatcagtttacttcatttcaaaactatttccaatcacaaggaaagatttatttaaaatatactcgtacatttcacctgtggatgtctataacttcatcctcagtatgttcccaaatctgtgctggcattgaaaggacaaaacattatactagtgggtttttctactaattattttttgaagcattattttcccaacacaaaagagcttttttctcggtataatgaaaattgaaatcctatgtgtattcaatagtaaatagacaaattttattttttatttccacttgaagagttacatttcgtataaaagtttacaaataacggtttttattttgattttttcagtataaaaaaagttgccttgatggcatattatgatgtaatgctaattgcttgtaggatagtaaatggtcagtattgaaacctaatctctagctgccgtcttgtagatatgaacgaatgttcaccaagcatgtattttgtattttgttgcattgtacactgcaactaataagccaaggaatcgacatatattaggtgcgtgtactgtttctaaaaaccacaaactaagaatgataaattatcaatatagtttagtatttgctaattttactacactcttttgttatgtatatgtagggaagtcatagggattataaattcaatttgagtaaaatttaaaaccatatattttatgataaagggcctttaacttaagatggccaaagcactgatattatatatttgctgtaaagagaattataagagttttatttttctgatattaaaagttacttaataaagacttgtttccattaacttg

SEQ ID NO: 23 - protein, 659 aa, Homo sapiens
MPSGSSAALALAAAPAPLPQPPPPPPPPPPPLPPPSGGPELEGDGLLLRERLAALGLDDPSPAEPGAPALRAPAAAAQGQARRAAELSPEERAPPGRPGAPEAAELELEEDEEEGEEAELDGDLLEEEELEEAEEEDRSSLLLLSPPAATASQTQQIPGGSLGSVLLPAARFDAREAAAAAAAAGVLYGGDDAQGMMAAMLSHAYGPGGCGAAAAALNGEQAALLRRKSVNTTECVPVPSSEHVAEIVGRQGCKIKALRAKTNTYIKTPVRGEEPIFVVTGRKEDVAMAKREILSAAEHFSMIRASRNKNGPALGGLSCSPNLPGQTTVQVRVPYRVVGLVVGPKGATIKRIQQQTHTYIVTPSRDKEPVFEVTGMPENVDRAREEIEMHIAMRTGNYIELNEENDFHYNGTDVSFEGGTLGSAWLSSNPVPPSRARMISNYRNDSSSSLGSGSTDSYFGSNRLADFSPTSPFSTGNFWFGDTLPSVGSEDLAVDSPAFDSLPTSAQTIWTPFEPVNPLSGFGSDPSGNMKTQRRGSQPSTPRLSPTFPESIEHPLARRVRSDPPSTGNHVGLPIYIPAFSNGTNSYSSSNGGSTSSSPPESRRKHDCVICFENEVIAALVPCGHNLFCMECANKICEKRTPSCPVCQTAVTQAIQIHS

SEQ ID NO: 24 - DNA, 3779 bp, Homo sapiens
atgcccagcggcagctccgcggccctggccctggcggcggccccggcccccctgccgcagccgcccccgccgccgccgccgccaccgccgcctctgccgccgccctcgggcggcccggagctcgagggggacgggctcctgctgagggagcgcttggccgcgctaggcctcgacgaccccagcccggcggagcccggcgccccggcgcttcgggccccggcagcggcggcgcagggccaggcccggcgggcggcggagctgtctccagaggagcgggctccgcccggccggcccggggccccggaggcggccgagctggagctggaagaggacgaggaggagggggaggaagcggagctggacggagacctgctggaggaggaggagctggaggaagcagaggaggaggaccggtcgtcgctgctgctgctgtcgccgcccgcggccaccgcctctcagacccagcagatcccgggcgggtccctggggtctgtgctgctgccagccgccaggttcgatgcccgggaggcggcggccgcggcggcggcggcgggggtgctgtacggaggggacgatgcccagggcatgatggcggcgatgctgtcccacgcctacggccccggcggttgtggggcggcggcggccgccctgaacggggagcaggcggccctgctccggagaaagagcgtcaacaccaccgagtgcgtcccggtgcccagctccgagcacgtcgccgagatcgtcggccgccagggttgtaaaattaaagcactgagagccaagacaaacacgtatatcaagactcctgttcgtggtgaagagcccatttttgttgtcactggaaggaaagaagatgttgccatggccaaaagagagatcctctcagctgcagagcacttctccatgattcgtgcatctcgaaacaaaaatgggcctgccctgggaggattatcatgtagtcctaatctgcccggtcaaaccaccgtccaagtcagggtcccttatcgtgtggtaggattagtggttggacccaaaggagcaactattaaaagaattcagcagcagacccacacctacatagtaactccgagcagagataaggaacctgtctttgaagtgacagggatgcctgaaaatgttgaccgagcacgggaagaaatagaaatgcatattgccatgcgtacaggaaactatatagagctcaatgaagagaatgatttccattacaatggtaccgatgtaagctttgaaggtggcactcttggctctgcgtggctctcctccaatcctgttcctcctagccgcgcaagaatgatatccaattatcgaaatgatagttccagttctctaggaagtggctctacagattcctactttggaagcaataggctggctgactttagtccaacaagcccatttagcacaggaaacttctggtttggagatacactaccatctgtaggctcagaagacctagcagttgactctcctgcctttgactctttaccaacatctgctcaaactatctggactccatttgaaccagttaacccactctctggctttgggagtgatccttctggtaacatgaagactcagcgcagaggaagtcagccatctactcctcgtctgtctcctacatttcctgagagcatagaacatccacttgctcggagggttaggagcgacccacctagtacaggcaaccatgttggccttccaatatatatccctgctttttctaatggtaccaatagttactcctcttccaatggtggttccacctctagctcacctccagaatcaagacgaaagcacgactgtgtgatttgctttgagaatgaggttattgctgccctagttccatgtggccacaacctcttctgcatggaatgtgccaacaagatctgtgaaaagagaacgccatcatgtccagtttgccagacagctgttactcaggcaatccaaattcactcttaactatatatatatacataaatactatatctctatatggactcgtaaaggcatgggtataatggtaccccccagtaaacttcctaatgatttcttatgactgttatcaggctttattgggattaggctaaagttgttagtaaacttataaaaggctgctatggtaacactaaacctaagtggtctcttgtctattagtttggtttgaattattagtactatcctgtagacccagagacatagtttatataagaattgctaaagctgaagttcaacttggctgagtgaagataatcataggttgtgtgagcctatgaaaaagtgtatacgtctaagatttcaaaacaatgggtcccaaagcctaaccactttaagagtttatggagggtacttggcattacagacgattcatacacttccagtgctgccttctttacactgccagttttgacaaaacaggtttgttttttattttacaacaacatatgcctaattctgcaggattgcaagtaactttttaatgcattgtgattacttattggtaatgatagggctgatggcagtttactagatcactggttataatttgggacaaaaactgctacatcaactttcatctcgcccagagtgctcaaggctggtatgatcagtggatcaggaatgcaattgtgaattcctgcccattgcctctcttggtgaatgtggaaatggccacctgggttttcccatatcaggaagggctttgggatggcacctatattggctgataattgaggatgcaaacattccattcattagtgtgatcgagctgttaatttttagactatagatcaaaatgtgaaacattttatgttcaatccatatttgtcttgcacattataaatatatttttattttttagtaatttaggggagggaggagggagaaagggataatgatgcccttggcataattcacaaaagcagctgtgacaacctccaatcagtttacttcatttcaaaactatttccaatcacaaggaaagatttatttaaaatatactcgtacatttcacctgtggatgtctataacttcatcctcagtatgttcccaaatctgtgctggcattgaaaggacaaaacattatactagtgggtttttctactaattattttttgaagcattattttcccaacacaaaagagcttttttctcggtataatgaaaattgaaatcctatgtgtattcaatagtaaatagacaaattttattttttatttccacttgaagagttacatttcgtataaaagtttacaaataacggtttttattttgattttttcagtataaaaaaagttgccttgatggcatattatgatgtaatgctaattgcttgtaggatagtaaatggtcagtattgaaacctaatctctagctgccgtcttgtagatatgaacgaatgttcaccaagcatgtattttgtattttgttgcattgtacactgcaactaataagccaaggaatcgacatatattaggtgcgtgtactgtttctaaaaaccacaaactaagaatgataaattatcaatatagtttagtatttgctaattttactacactcttttgttatgtatatgtagggaagtcatagggattataaattcaatttgagtaaaatttaaaaccatatattttatgataaagggcctttaacttaagatggccaaagcactgatattatatatttgctgtaaagagaattataagagttttatttttctgatattaaaagttacttaataaagacttgtttccattaacttg

SEQ ID NO: 25 - DNA, 3770 bp, Homo sapiens
atgcccagcggcagctccgcggccctggccctggcggcggccccggcccccctgccgcagccgcccccgccgccgccgccgccaccgccgcctctgccgccgccctcgggcggcccggagctcgagggggacgggctcctgctgagggagcgcttggccgcgctaggcctcgacgaccccagcccggcggagcccggcgccccggcgcttcgggccccggcagcggcggcgcagggccaggcccggcgggcggcggagctgtctccagaggagcgggctccgcccggccggcccggggccccggaggcggccgagctggagctggaagaggacgaggaggagggggaggaagcggagctggacggagacctgctggaggaggaggagctggaggaagcagaggaggaggaccggtcgtcgctgctgctgctgtcgccgcccgcggccaccgcctctcagacccagcagatcccgggcgggtccctggggtctgtgctgctgccagccgccaggttcgatgcccgggaggcggcggcggcggcgggggtgctgtacggaggggacgatgcccagggcatgatggcggcgatgctgtcccacgcctacggccccggcggttgtggggcggcggcggccgccctgaacggggagcaggcggccctgctccggagaaagagcgtcaacaccaccgagtgcgtcccggtgcccagctccgagcacgtcgccgagatcgtcggccgccagggttgtaaaattaaagcactgagagccaagacaaacacgtatatcaagactcctgttcgtggtgaagagcccatttttgttgtcactggaaggaaagaagatgttgccatggccaaaagagagatcctctcagctgcagagcacttctccatgattcgtgcatctcgaaacaaaaatgggcctgccctgggaggattatcatgtagtcctaatctgcccggtcaaaccaccgtccaagtcagggtcccttatcgtgtggtaggattagtggttggacccaaaggagcaactattaaaagaattcagcagcagacccacacctacatagtaactccgagcagagataaggaacctgtctttgaagtgacagggatgcctgaaaatgttgaccgagcacgggaagaaatagaaatgcatattgccatgcgtacaggaaactatatagagctcaatgaagagaatgatttccattacaatggtaccgatgtaagctttgaaggtggcactcttggctctgcgtggctctcctccaatcctgttcctcctagccgcgcaagaatgatatccaattatcgaaatgatagttccagttctctaggaagtggctctacagattcctactttggaagcaataggctggctgactttagtccaacaagcccatttagcacaggaaacttctggtttggagatacactaccatctgtaggctcagaagacctagcagttgactctcctgcctttgactctttaccaacatctgctcaaactatctggactccatttgaaccagttaacccactctctggctttgggagtgatccttctggtaacatgaagactcagcgcagaggaagtcagccatctactcctcgtctgtctcctacatttcctgagagcatagaacatccacttgctcggagggttaggagcgacccacctagtacaggcaaccatgttggccttccaatatatatccctgctttttctaatggtaccaatagttactcctcttccaatggtggttccacctctagctcacctccagaatcaagacgaaagcacgactgtgtgatttgctttgagaatgaggttattgctgccctagttccatgtggccacaacctcttctgcatggaatgtgccaacaagatctgtgaaaagagaacgccatcatgtccagtttgccagacagctgttactcaggcaatccaaattcactcttaactatatatatatacataaatactatatctctatatggactcgtaaaggcatgggtataatggtaccccccagtaaacttcctaatgatttcttatgactgttatcaggctttattgggattaggctaaagttgttagtaaacttataaaaggctgctatggtaacactaaacctaagtggtctcttgtctattagtttggtttgaattattagtactatcctgtagacccagagacatagtttatataagaattgctaaagctgaagttcaacttggctgagtgaagataatcataggttgtgtgagcctatgaaaaagtgtatacgtctaagatttcaaaacaatgggtcccaaagcctaaccactttaagagtttatggagggtacttggcattacagacgattcatacacttccagtgctgccttctttacactgccagttttgacaaaacaggtttgttttttattttacaacaacatatgcctaattctgcaggattgcaagtaactttttaatgcattgtgattacttattggtaatgatagggctgatggcagtttactagatcactggttataatttgggacaaaaactgctacatcaactttcatctcgcccagagtgctcaaggctggtatgatcagtggatcaggaatgcaattgtgaattcctgcccattgcctctcttggtgaatgtggaaatggccacctgggttttcccatatcaggaagggctttgggatggcacctatattggctgataattgaggatgcaaacattccattcattagtgtgatcgagctgttaatttttagactatagatcaaaatgtgaaacattttatgttcaatccatatttgtcttgcacattataaatatatttttattttttagtaatttaggggagggaggagggagaaagggataatgatgcccttggcataattcacaaaagcagctgtgacaacctccaatcagtttacttcatttcaaaactatttccaatcacaaggaaagatttatttaaaatatactcgtacatttcacctgtggatgtctataacttcatcctcagtatgttcccaaatctgtgctggcattgaaaggacaaaacattatactagtgggtttttctactaattattttttgaagcattattttcccaacacaaaagagcttttttctcggtataatgaaaattgaaatcctatgtgtattcaatagtaaatagacaaattttattttttatttccacttgaagagttacatttcgtataaaagtttacaaataacggtttttattttgattttttcagtataaaaaaagttgccttgatggcatattatgatgtaatgctaattgcttgtaggatagtaaatggtcagtattgaaacctaatctctagctgccgtcttgtagatatgaacgaatgttcaccaagcatgtattttgtattttgttgcattgtacactgcaactaataagccaaggaatcgacatatattaggtgcgtgtactgtttctaaaaaccacaaactaagaatgataaattatcaatatagtttagtatttgctaattttactacactcttttgttatgtatatgtagggaagtcatagggattataaattcaatttgagtaaaatttaaaaccatatattttatgataaagggcctttaacttaagatggccaaagcactgatattatatatttgctgtaaagagaattataagagttttatttttctgatattaaaagttacttaataaagacttgtttccattaacttg

SEQ ID NO: 26 - protein, 659 aa, Homo sapiens; identical to SEQ ID NO: 23

SEQ ID NO: 27 - protein, 656 aa, Homo sapiens [sequence omitted]

SEQ ID NO: 28 - DNA, 21 bp, Artificial Sequence
  siRNA
ccaccguccaagucagggutt

SEQ ID NO: 29 - DNA, 21 bp, Artificial Sequence
  siRNA
acccugacuuggacgguggtt

SEQ ID NO: 30 - DNA, 21 bp, Artificial Sequence
  siRNA
ugauaguuccaguucucuatt

SEQ ID NO: 31 - DNA, 21 bp, Artificial Sequence
  siRNA
uagagaacuggaacuaucatt

SEQ ID NO: 32 - DNA, 21 bp, Artificial Sequence
  siRNA
uaguuccaguucucuaggatt

SEQ ID NO: 33 - DNA, 21 bp, Artificial Sequence
  siRNA
uccuagagaacuggaacuatt

SEQ ID NO: 34 - DNA, 21 bp, Artificial Sequence
  siRNA
ggaaguggcucuacagauutt

SEQ ID NO: 35 - DNA, 21 bp, Artificial Sequence
  siRNA
aaucuguagagccacuucctt

SEQ ID NO: 36 - DNA, 21 bp, Artificial Sequence
  siRNA
guggcucuacagauuccuatt

SEQ ID NO: 37 - DNA, 21 bp, Artificial Sequence
  siRNA
uaggaaucuguagagccactt

SEQ ID NO: 38 - DNA, 21 bp, Artificial Sequence
  siRNA
cuuuaguccaacaagcccatt

SEQ ID NO: 39 - DNA, 21 bp, Artificial Sequence
  siRNA
ugggcuuguuggacuaaagtt

SEQ ID NO: 40 - DNA, 21 bp, Artificial Sequence
  siRNA
guccaacaagcccauuuagtt

SEQ ID NO: 41 - DNA, 21 bp, Artificial Sequence
  siRNA
cuaaaugggcuuguuggactt

SEQ ID NO: 42 - DNA, 21 bp, Artificial Sequence
  siRNA
agcccauuuagcacaggaatt

SEQ ID NO: 43 - DNA, 21 bp, Artificial Sequence
  siRNA
uuccugugcuaaaugggcutt

SEQ ID NO: 44 - DNA, 21 bp, Artificial Sequence
  siRNA
gcccauuuagcacaggaaatt

SEQ ID NO: 45 - DNA, 21 bp, Artificial Sequence
  siRNA
uuuccugugcuaaaugggctt

SEQ ID NO: 46 - DNA, 21 bp, Artificial Sequence
  siRNA
accaguuaacccacucucutt

SEQ ID NO: 47 - DNA, 21 bp, Artificial Sequence
  siRNA
agagaguggguuaacuggutt

SEQ ID NO: 48 - DNA, 21 bp, Artificial Sequence
  siRNA
ccauguuggccuuccaauatt

SEQ ID NO: 49 - DNA, 21 bp, Artificial Sequence
  siRNA
uauuggaaggccaacauggtt

SEQ ID NO: 50 - protein, 737 aa, Artificial Sequence
  fusion protein
MPSGSSAALALAAAPAPLPQPPPPPPPPPPPLPPPSGGPELEGDGLLLRERLAALGLDDPSPAEPGAPALRAPAAAAQGQARRAAELSPEERAPPGRPGAPEAAELELEEDEEEGEEAELDGDLLEEEELEEAEEEDRSSLLLLSPPAATASQTQQIPGGSLGSVLLPAARFDAREAAAAAGVLYGGDDAQGMMAAMLSHAYGPGGCGAAAAALNGEQAALLRRKSVNTTECVPVPSSEHVAEIVGRQGCKIKALRAKTNTYIKTPVRGEEPIFVVTGRKEDVAMAKREILSAAEHFSMIRASRNKNGPALGGLSCSPNLPGQTTVQVRVPYRVVGLVVGPKGATIKRIQQQTHTYIVTPSRDKEPVFEVTGMPENVDRAREEIEMHIAMRTGNYIELNEENDFHYNGTDVSFEGGTLGSAWLSSNPVPPSRARMISNYRNDSSSSLGSGSTDSYFGSNRLADFSPTSPFSTGNFWFGDTLPSVGSEDLAVDSPAFDSLPTSAQTIWTPFEPVNPLSGFGSDPSGNMKTQRRGSQPSTPRLSPTFPESIEHPLARRVRSDPPSTGNHVGLPIYIPAFSNGTNSYSSSNGGSTSSSPPESRRKHDCVICFENEVIAALVPCGHNLFCMECANKICEKRTPSCPVCQTAVTQAIQIHSMLIKVKTLTGKEIEIDIEPTDKVERIKERVEEKEGIPPQQQRLIYSGKQMNDEKTAADYKILGGSVLHLVLALRGGGGLRQ

SEQ ID NO: 51 - protein, 545 aa, Artificial Sequence
  fusion protein
MMAAMLSHAYGPGGCGAAAAALNGEQAALLRRKSVNTTECVPVPSSEHVAEIVGRQGCKIKALRAKTNTYIKTPVRGEEPIFVVTGRKEDVAMAKREILSAAEHFSMIRASRNKNGPALGGLSCSPNLPGQTTVQVRVPYRVVGLVVGPKGATIKRIQQQTHTYIVTPSRDKEPVFEVTGMPENVDRAREEIEMHIAMRTGNYIELNEENDFHYNGTDVSFEGGTLGSAWLSSNPVPPSRARMISNYRNDSSSSLGSGSTDSYFGSNRLADFSPTSPFSTGNFWFGDTLPSVGSEDLAVDSPAFDSLPTSAQTIWTPFEPVNPLSGFGSDPSGNMKTQRRGSQPSTPRLSPTFPESIEHPLARRVRSDPPSTGNHVGLPIYIPAFSNGTNSYSSSNGGSTSSSPPESRRKHDCVICFENEVIAALVPCGHNLFCMECANKICEKRTPSCPVCQTAVTQAIQIHSMLIKVKTLTGKEIEIDIEPTDKVERIKERVEEKEGIPPQQQRLIYSGKQMNDEKTAADYKILGGSVLHLVLALRGGGGLRQ

SEQ ID NO: 52 - protein, 737 aa, Artificial Sequence
  fusion protein
MLIKVKTLTGKEIEIDIEPTDKVERIKERVEEKEGIPPQQQRLIYSGKQMNDEKTAADYKILGGSVLHLVLALRGGGGLRQMPSGSSAALALAAAPAPLPQPPPPPPPPPPPLPPPSGGPELEGDGLLLRERLAALGLDDPSPAEPGAPALRAPAAAAQGQARRAAELSPEERAPPGRPGAPEAAELELEEDEEEGEEAELDGDLLEEEELEEAEEEDRSSLLLLSPPAATASQTQQIPGGSLGSVLLPAARFDAREAAAAAGVLYGGDDAQGMMAAMLSHAYGPGGCGAAAAALNGEQAALLRRKSVNTTECVPVPSSEHVAEIVGRQGCKIKALRAKTNTYIKTPVRGEEPIFVVTGRKEDVAMAKREILSAAEHFSMIRASRNKNGPALGGLSCSPNLPGQTTVQVRVPYRVVGLVVGPKGATIKRIQQQTHTYIVTPSRDKEPVFEVTGMPENVDRAREEIEMHIAMRTGNYIELNEENDFHYNGTDVSFEGGTLGSAWLSSNPVPPSRARMISNYRNDSSSSLGSGSTDSYFGSNRLADFSPTSPFSTGNFWFGDTLPSVGSEDLAVDSPAFDSLPTSAQTIWTPFEPVNPLSGFGSDPSGNMKTQRRGSQPSTPRLSPTFPESIEHPLARRVRSDPPSTGNHVGLPIYIPAFSNGTNSYSSSNGGSTSSSPPESRRKHDCVICFENEVIAALVPCGHNLFCMECANKICEKRTPSCPVCQTAVTQAIQIHS

SEQ ID NO: 53 - protein, 545 aa, Artificial Sequence
  fusion protein
MLIKVKTLTGKEIEIDIEPTDKVERIKERVEEKEGIPPQQQRLIYSGKQMNDEKTAADYKILGGSVLHLVLALRGGGGLRQMMAAMLSHAYGPGGCGAAAAALNGEQAALLRRKSVNTTECVPVPSSEHVAEIVGRQGCKIKALRAKTNTYIKTPVRGEEPIFVVTGRKEDVAMAKREILSAAEHFSMIRASRNKNGPALGGLSCSPNLPGQTTVQVRVPYRVVGLVVGPKGATIKRIQQQTHTYIVTPSRDKEPVFEVTGMPENVDRAREEIEMHIAMRTGNYIELNEENDFHYNGTDVSFEGGTLGSAWLSSNPVPPSRARMISNYRNDSSSSLGSGSTDSYFGSNRLADFSPTSPFSTGNFWFGDTLPSVGSEDLAVDSPAFDSLPTSAQTIWTPFEPVNPLSGFGSDPSGNMKTQRRGSQPSTPRLSPTFPESIEHPLARRVRSDPPSTGNHVGLPIYIPAFSNGTNSYSSSNGGSTSSSPPESRRKHDCVICFENEVIAALVPCGHNLFCMECANKICEKRTPSCPVCQTAVTQAIQIHS

SEQ ID NO: 54 - DNA, 87 bp, Artificial Sequence
  primer
ccggggatccggcatgatggcggcgatgctgtcccacgcctacggccccggcggttgtggggcggcggcagccgccctgaacgggga

SEQ ID NO: 55 - DNA, 20 bp, Artificial Sequence
  primer
ggtgtgggtctgctgctgaa

SEQ ID NO: 56 - DNA, 19 bp, Artificial Sequence
  primer
ccatgattcgtgcatctcg

SEQ ID NO: 57 - DNA, 37 bp, Artificial Sequence
  primer
ccggtctagactcgagagagtgaatttggattgcctg

SEQ ID NO: 58 - DNA, 33 bp, Artificial Sequence
  primer
ccggggatccgaaatgatggcggcgatgctgtc

SEQ ID NO: 59 - DNA, 21 bp, Homo sapiens
aatgatagttccagttctcta

SEQ ID NO: 60 - DNA, 21 bp, Homo sapiens
gatagttccagttctctagga

SEQ ID NO: 61 - DNA, 21 bp, Homo sapiens
taggaagtggctctacagatt

SEQ ID NO: 62 - DNA, 21 bp, Homo sapiens
aagtggctctacagattccta

SEQ ID NO: 63 - DNA, 21 bp, Homo sapiens
gactttagtccaacaagccca

SEQ ID NO: 64 - DNA, 21 bp, Homo sapiens
tagtccaacaagcccatttag

SEQ ID NO: 65 - DNA, 21 bp, Homo sapiens
caagcccatttagcacaggaa

SEQ ID NO: 66 - DNA, 21 bp, Homo sapiens
aagcccatttagcacaggaaa

SEQ ID NO: 67 - DNA, 21 bp, Homo sapiens
gaaccagttaacccactctct

SEQ ID NO: 68 - DNA, 21 bp, Homo sapiens
aaccatgttggccttccaata

SEQ ID NO: 69 - protein, 7 aa, Artificial Sequence
  motif
RPDPTAP

SEQ ID NO: 70 - protein, 7 aa, Artificial Sequence
  motif
RPLPVAP

SEQ ID NO: 71 - protein, 7 aa, Artificial Sequence
  motif
RPEPTAP

SEQ ID NO: 72 - protein, 4 aa, Artificial Sequence
  motif
YEDL

SEQ ID NO: 73 - protein, 7 aa, Artificial Sequence
  motif
PTAPPEY

SEQ ID NO: 74 - protein, 10 aa, Artificial Sequence
  motif
RPEPTAPPEE

(19) United States
(12) Patent Application Publication
(10) Pub. No.: US 2007/0141716 A1
(43) Pub. Date: Jun. 21, 2007

(54) Pem-3-like compositions and related methods thereof

(76) Inventors: Tsvika Greener, Ness-Ziona (IL); Danny Ben-Avraham, Zichron Jackov (IL)

Correspondence Address: FISH & NEAVE IP GROUP;ROPES & GRAY LLP, ONE INTERNATIONAL PLACE, BOSTON, MA, 02110-2624, US

(21) Appl. No.: 10/559,011

(22) PCT Filed: May 28, 2004
(86) PCT No.: PCT/US04/16865

§ 371 Date: Jul. 5, 2006

Related U.S. Application Data
(60) Provisional application No. 60/474,474, filed on May 30, 2003.
(60) Provisional application No. 60/530,833, filed on Dec. 18, 2003.
(60) Provisional application No. 60/537,310, filed on Jan. 16, 2004.

(51) Int. Cl. G01N 33/00

(52) U.S. Cl. 436/89

(57) ABSTRACT

The application discloses methods and compositions relating to PEM-3-like polypeptides and nucleic acids involved in a variety of biological processes, including viral reproduction.

### BACKGROUND

Potential drug target validation involves determining whether a DNA, RNA or protein molecule is implicated in a disease process and is therefore a suitable target for development of new therapeutic drugs. Drug discovery, the process by which bioactive compounds are identified and characterized, is a critical step in the development of new treatments for human diseases. The landscape of drug discovery has changed dramatically due to the genomics revolution. DNA and protein sequences are yielding a host of new drug targets and an enormous amount of associated information.

The identification of genes and proteins involved in various disease states or key biological processes, such as inflammation and immune response, is a vital part of the drug design process. Many diseases and disorders could be treated or prevented by decreasing the expression of one or more genes involved in the molecular etiology of the condition if the appropriate molecular target could be identified and appropriate antagonists developed. For example, cancer, in which one or more cellular oncogenes become activated and result in the unchecked progression of cell cycle processes, could be treated by antagonizing appropriate cell cycle control genes. Furthermore many human genetic diseases, such as Huntington's disease, and certain prior conditions, which are influenced by both genetic and epigenetic factors, result from the inappropriate activity of a polypeptide as opposed to the complete loss of its function. Accordingly, antagonizing the aberrant function of such mutant genes would provide a means of treatment. Additionally, infectious diseases such as HIV have been successfully treated with molecular antagonists targeted to specific essential retroviral proteins such as HIV protease or reverse transcriptase. Drug therapy strategies for treating such diseases and disorders have frequently employed molecular antagonists which target the polypeptide product of the disease gene(s). However the discovery of relevant gene or protein targets is often difficult and time consuming.

One area of particular interest is the identification of host genes and proteins that are co-opted by viruses during the viral life cycle. The serious and incurable nature of many viral diseases, coupled with the high rate of mutations found in many viruses, makes the identification of antiviral agents a high priority for the improvement of world health. Genes and proteins involved in a viral life cycle are also appealing as a subject for investigation because such genes and proteins will typically have additional activities in the host cell and may play a role in other non-viral disease states.

Viral maturation requires the proteolytic processing of the Gag proteins and the activity of the host proteins. It is believed that cellular machineries for exo/endocytosis and for ubiquitin conjugation may be involved in the maturation. In particular, the assembly, budding and subsequent release of retroid viruses and RNA viruses such as various retroviruses, rhabdoviruses, lentiviruses, and filoviruses depends on the Gag polyprotein. After its synthesis, Gag is targeted to the plasma membrane where it induces budding of nascent virus particles.

The role of ubiquitin in virus assembly was suggested by Dunigan et al. (1988, Virology 165, 310, Meyers et al. 1991, Virology 180, 602), who observed that mature virus particles were enriched in unconjugated ubiquitin. More recently, it was shown that proteasome inhibitors suppress the release of HIV-1, HIV-2 and virus-like particles derived from SIV and RSV Gag. Also, inhibitors affect Gag processing and maturation into infectious particles (Schubert et al 2000, PNAS 97, 13057, Harty et al. 2000, PNAS 97, 13871, Strack et al. 2000, PNAS 97, 13063, Patnaik et al. 2000, PNAS 97, 13069).

It is well known in the art that ubiquitin-mediated proteolysis is the major pathway for the selective, controlled degradation of intracellular proteins in eukaryotic cells. Ubiquitin modification of a variety of protein targets within the cell appears to be important in a number of basic cellular functions such as regulation of gene expression, regulation of the cell-cycle, modification of cell surface receptors, biogenesis of ribosomes, and DNA repair. One major function of the ubiquitin-mediated system is to control the half-lives of cellular proteins. The half-life of different proteins can range from a few minutes to several days, and can vary considerably depending on the cell-type, nutritional and environmental conditions, as well as the stage of the cell-cycle.

Targeted proteins undergoing selective degradation, presumably through the actions of a ubiquitin-dependent proteosome, are covalently tagged with ubiquitin through the formation of an isopeptide bond between the C-terminal glycyl residue of ubiquitin and a specific lysyl residue in the substrate protein. This process is catalyzed by a ubiquitin-activating enzyme (E1) and a ubiquitin-conjugating enzyme (E2), and in some instances may also require auxiliary substrate recognition proteins (E3s). Following the linkage of the first ubiquitin chain, additional molecules of ubiquitin may be attached to lysine side chains of the previously conjugated moiety to form branched multi-ubiquitin chains.

The conjugation of ubiquitin to protein substrates is a multi-step process. In an initial ATP requiring step, a thioester is formed between the C-terminus of ubiquitin and an internal cysteine residue of an E1 enzyme. Activated ubiquitin is then transferred to a specific cysteine on one of several E2 enzymes. Finally, these E2 enzymes donate ubiquitin to protein substrates. Substrates are recognized either directly by ubiquitin-conjugated enzymes or by associated substrate recognition proteins, the E3 proteins, also known as ubiquitin ligases.

The vesicular trafficking systems are the major pathways for the distribution of proteins among cell organelles, the plasma membrane and the extracellular medium. The vesicular trafficking systems may be directly or indirectly involved in a variety of disease states. The major vesicle trafficking systems in eukaryotic cells include those systems that are mediated by clathrin-coated vesicles and coatomer-coated vesicles. Clathrin-coated vesicles are generally involved in transport, such as in the case of receptor mediated endocytosis, between the plasma membrane and the early endosomes, as well as from the trans-Golgi network to endosomes. Coatomer-coated vesicles include coat protein I (COP-I) coated vesicles and COP-II coated vesicles, both of which tend to mediate transport of a variety of molecules between the ER and Golgi cisternae. In each case, a vesicle is formed by budding out from a portion of membrane that is coated with coat proteins, and the vesicle sheds its coat prior to fusing with the target membrane.

Clathrin coats assemble on the cytoplasmic face of a membrane, forming pits that ultimately pinch off to become vesicles. Clathirin itself is composed of two subunits, the clathrin heavy chain and the clathrin light chain, that form the clathrin triskelion. Clathrins associate with a host of other proteins, including the assembly protein, AP180, the adaptor complexes (AP1, AP2, AP3 and AP4), beta-arrestin, arrestin 3, auxilin, epsin, Eps15, v-SNAREs, amphiphysins, dynamin, synaptojanin and endophilin. The adaptor complexes promote clathrin cage formation, and help connect clathrin up to the membrane, membrane proteins, and many of the preceding components. API associates with clathrin coated vesicles derived from the trans-Golgi network and contains γ, β, μ1 and σ1 polypeptide chains. AP2 associates with endocytic clathrin coated vesicles and contains α, β2, μ2, and σ2 polypeptides. Interactions between the clathrin complex and other proteins are mediated by a variety of domains found in the complex proteins, such as SH3 (Src homology 3) domains, PH (pleckstrin homology) domains, EH domains and NPF domains. (Marsh et al. (1999) Science 285:215-20; Pearse et al. (2000) Curr Opin Struct Biol 10(2):220-8).

Coatomer-coated vesicle formation is initiated by recruitment of a small GTPase (e.g., ARF or SAR) by its cognate guanine nucleotide excahnge factor (e.g., SEC12, GEA1, GEA2). The initial complex is recognized by a coat protein complex (COPI or COPII). The coat then grows across the membrane, and various cargo proteins become entrapped in the growing network. The membrane ultimately bulges and becomes a vesicle. The coat proteins stimulate the GTPase activity of the GTPase, and upon hydrolysis of the GTP, the coat proteins are released from the complex, uncoating the vesicle. Other proteins associated with coatomer coated vesicles include v-SNAREs, Rab GTPases and various receptors that help recruit the appropriate cargo proteins. (Springer et al. (1999) Cell 97:145-48).

### SUMMARY

In certain aspects, the invention relates to novel PEM-3-like nucleic acids and proteins encoded thereby. In certain aspects, the invention relates to methods and compositions employing human PEM-3-like nucleic acids and proteins. In certain embodiments, PEM-3-like proteins play a role in viral maturation. Optionally, PEM-3-like protein acts in the assembly or trafficking of complexes that mediate viral release. In one embodiment, PEM-3-like polypeptides may stimulate ubiquitination of certain proteins or stimulate membrane fusion or both. As one of skill in the art can readily appreciate, a PEM-3-like protein may form multiple different complexes at different times.

Described herein are methods for identifying an antiviral agent comprising: (a) providing a PEM-3-like polypeptide and a test agent; and (b) identifying a test agent that interacts with the PEM-3-like polypeptide. In certain embodiments, the PEM-3-like polypeptide is selected from the group consisting of: SEQ ID NOS: 2, 4, 6, 8, 10, 12, 14, 16, 18, 20, 23, 26 and 27 and fragments comprising at least 20 consecutive amino acids of any of SEQ ID NOS: 2, 4, 6, 8, 10, 12, 14, 16, 18, 20, 23, 26 and 27. In certain further embodiments, the PEM-3-like polypeptide is expressed in a cell. In additional embodiments, the PEM-3 -like polypeptide is a purified polypeptide. In a preferred embodiment, the PEM-3-like polypeptide comprises a domain selected from the group consisting of: a KH domain and a RING domain. In certain embodiments, the test agent binds to a domain selected from the group consisting of: a KH domain and a RING domain. In further embodiments, the test agent is a polypeptide, an antibody, a small molecule, or a peptidomiinetic.

In additional embodiments, the application relates to methods for identifying an antiviral agent comprising: (a) providing a PEM-3-like nucleic acid and a test agent; and (b) identifying a test agent that binds to the PEM-3-like nucleic acid. In certain further embodiments, the PEM-3-like nucleic acid is selected from the group consisting of SEQ ID NOS: 1, 3, 5, 7, 9, 11, 13, 15, 17, 19, 22, 24 and 25. In additional embodiments, the test agent is selected from the group consisting of: a ribonucleic acid, an antisense oligonucleotide, an RNAi construct, a DNA enzyme, and a ribozyme. In further embodiments, the binding of the test agent to said PEM-3-like nucleic acid decreases the level of a PEM-3-like transcript. In additional embodiments, the methods of the present application further comprise administering a composition comprising the test agent to a cell transfected with at least a portion of a viral genome and measuring the effect of the test agent on the production of viral or virus-like particles. In other embodiments, the antiviral agent is effective against a virus selected from the group consisting of: an envelope virus, a retroid virus and a RNA virus. In certain embodiments, a) the retroid virus is a lentivirus; b) the retroid virus is an HIV1 lentivirus; c) the RNA virus is a filovirus; or d) the RNA virus is an ebola filovirus.

The present application further relates to a method for inhibiting infection in a subject in need thereof, comprising administering an effective amount of an agent that inhibits a PEM-3-like protein activity. In certain embodiments, the agent inhibits the ubiquitin ligase activity of the PEM-3-like polypeptide. In additional embodiments, the agent is selected from the group consisting of: a small molecule, an antibody, a fragment of an antibody, a peptidomimetic, and a polypeptide. In yet other embodiments, the agent inhibits the interaction between a PEM-3-like polypeptide and a PEM-3-like-AP. In certain embodiments, the PEM-3-like-AP is selected from the group consisting of: an El, an E2, a PEM-3-like polypeptide, a ubiquitin, and a NEDD8. In certain aspects, the E2 is selected from the group consisting of: UBCH5, UBC13, and UBC12. In additional aspects, the El is APP-BP1/Uba3. In yet additional embodiments, the agent is selected from the group consisting of: an antisense oligonucleotide, an RNAi construct, a DNA enzyme, and a ribozyme. In certain embodiments, the agent decreases the level of PEM-3-like mRNA. Examples of RNAi constructs that may be used to target a PEM-3-like polypeptide include the nucleic acid sequences depicted in any of SEQ ID NOS: 28-49.

In certain embodiments, the application relates to an isolated antibody, or fragment thereof, specifically immunoreactive with an epitope of a sequence selected from the group consisting of: SEQ ID NOS: 2, 4, 6, 8, 10, 12, 14, 16, 18, 20, 23, 26 and 27. In certain embodiments, the antibody disrupts the interaction between a polypeptide of SEQ ID NOS: 2, 4, 6, 8, 10, 12, 14, 16, 18, 20, 23, 26 and 27 and a PEM-3-like-AP. In additional embodiments, said antibody is selected from the group consisting of: a polyclonal antibody, a monoclonal antibody, an Fab fragment and a single chain antibody. In additional embodiments, said antibody is labeled with a detectable label. In further embodiments, the PEM-3-like-AP is selected from the group consisting of: an El, an E2, a PEM-3-like polypeptide, a ubiquitin, and a NEDD8. Examples of E2s include UBCH5, UBC13, and UBC12. An example of an El is APP-BP1/Uba3. The present application also provides kits for detecting a human PEM-3-like polypeptide comprising (a) an antibody an isolated antibody, or fragment thereof, specifically immunoreactive with an epitope of a sequence selected from the group consisting of: SEQ ID NOS: 2, 4, 6, 8, 10, 12, 14, 16, 18, 20, 23, 26 and 27, and (b) a detectable label for detecting said antibody. In certain embodiments, the antibody disrupts the interaction between a polypeptide of SEQ ID NOS: 2, 4, 6, 8, 10, 12, 14, 16, 18, 20, 23, 26 and 27 and a PEM-3-like-AP.

The application further relates to a method of inhibiting viral maturation comprising inhibiting a ubiquitin-related activity of a PEM-3-like polypeptide. In certain embodiments, the PEM-3-like polypeptide is selected from the group consisting of: SEQ ID NOS: 2, 4, 6, 8, 10, 12, 14, 16, 18, 20, 23, 26 and 27 and fragments comprising at least 20 consecutive amino acids of any of SEQ ID NOS: 2, 4, 6, 8, 10, 12, 14, 16, 18, 20, 23, 26 and 27. In certain further embodiments, the method comprises inhibiting an activity of the RING domain of the PEM-3-like polypeptide. In additional embodiments, viral maturation is inhibited by administering an agent selected from the group consisting of: a small molecule, an antibody, a peptidomimetic, and a polypeptide. In yet other embodiments, viral maturation is inhibited by administering an agent selected from the group consisting of an antisense oligonucleotide, an RNAi construct, a DNA enzyme, and aribozyme. Examples of RNAi constructs include any of the nucleic acid sequences depicted in SEQ ID NOS: 28-49. In certain embodiments, the method comprises inhibiting viral maturation of a virus selected from the group consisting of: an envelope virus, a retroid virus or an RNA virus. In certain embodiments, (a) the retroid virus is a lentivirus; (b) the retroid virus is an HIV1 lentivirus; (c) the RNA virus is a filovirus; or (d) the RNA virus is an ebola filovirus.

The present application further relates to a method for testing a ubiquitin-related activity of a PEM-3-like polypeptide comprising: (a) forming a mixture compatible with the ubiquitin-related activity comprising: a ubiquitin; an El; an E2; and a PEM-3-like polypeptide; and (b) detecting whether said ubiquitin binds to said PEM-3-like polypeptide. In certain embodiments, the PEM-3-like polypeptide is selected from the group consisting of SEQ ID NOS: 2, 4, 6, 8, 10, 12, 14, 16, 18, 20, 23, 26 and 27. In additional embodiments, the mixture further comprises a PEM-3-like-AP. In certain embodiments, the method further comprises detecting whether said ubiquitin binds to said PEM-3-like-AP. In certain embodiments, the ubiquitin is detectably labeled. In additional embodiments, the PEM-3-like polypeptide is detectably labeled. In certain embodiments, a label is selected from the group consisting of: radioisotopes, fluorescent compounds, enzymes, and enzyme co-factors. In additional embodiments, the mixture further comprises NEDD8. In additional embodiments, the PEM-3-like polypeptide is neddylated. In yet other embodiments, the PEM-3-like polypeptide is a fusion protein comprising a NEDD8 polypeptide. In certain embodiments, the NEDD8 polypeptide is fused to the N-terminus of the PEM-3-like polypeptide. IN certain embodiments, the NEDD8 polypeptide is fused to the C-terminus of the PEM-3-like polypeptide. In additional embodiments, the PEM-3-like fusion protein comprises amino acid sequence selected from the group consisting of SEQ ID NOS: 50-53.

In additional embodiments, the present application provides an assay for identifying an inhibitor of a ubiquitin-related activity of a PEM-3-like polypeptide, comprising: (a) providing a ubiquitin-conjugating system comprising a ubiquitin; E2; and a PEM-3-like polypeptide, under conditions which promote ubiquitination of the PEM-3-like polypeptide; (b) contacting the ubiquitin-conjugating system with a test agent; (c) measuring a level of ubiquitination of the PEM-3-like polypeptide in the presence of the test agent; and (d) comparing the measured level of ubiquitination in the presence of the test agent with a suitable reference, wherein a decrease in ubiquitination of the PEM-3-like polypeptide in the presence of the candidate agent is indicative of an inhibitor of ubiquitination of the regulatory protein. In certain embodiments, the ubiquitin-conjugating system further comprises a PEM-3-like-AP. In certain embodiments, the ubiquitin is provided in a form selected from the group consisting of: an unconjugated ubiquitin, in which case the ubiquitin-conjugating system further comprises an E1 and adenosine triphosphate; an activated E1:ubiquitin conjugate; and an activated E2:ubiquitin thioester complex. In additional embodiments, the ubiquitin is detectably labeled. In other embodiments, the PEM-3-like polypeptide is detectably labeled. In further embodiments, the ubiquitin-conjugating system further comprises NEDD8. In certain embodiments, the PEM-3-like polypeptide is neddylated. In certain embodiments, the PEM-3-like polypeptide is a fusion protein comprising a NEDD8 polypeptide. In certain embodiments, the NEDD8 polypeptide is fused to the N-terminus of the PEM-3-like polypeptide. In certain embodiments, the NEDD8 polypeptide is fused to the C-terminus of the PEM-3-like polypeptide. In additional embodiments, the PEM-3-like fusion protein comprises amino acid sequence selected from the group consisting of: SEQ ID NOS: 50-53.

The subject application additionally relates to a therapeutic composition comprising an inhibitor of any one of a PEM-3-like polypeptide and a pharmaceutically acceptable excipient. In certain embodiments, the inhibitor is selected from the group consisting of: a small molecule, an antibody, a polypeptide, and a peptidomimetic. In certain embodiments, the inhibitor disrupts the interaction between a PEM-3-like polypeptide and PEM-3-like-AP and/or inhibits a ubiquitin-related activity of a PEM-3-like polypeptide. In additional embodiments, the inhibitor is selected from the group consisting of: an antisense oligonucleotide, a DNA enzyme, an RNAi construct, and a ribozyme. In certain further embodiments, the RNAi construct is selected from the group consisting of: SEQ ID NOS: 28-49.

In other embodiments, the application relates to a composition comprising a PEM-3-like polypeptide and ubiquitin. The present application additionally relates to a PEM-3-like polypeptide-ubiquitin conjugate. In certain embodiments, the application relates to a composition comprising a PEM-3-like polypeptide and a NEDD8 polypeptide. In yet other embodiments, the application relates to a PEM-3-like polypeptide-NEDD8 conjugate. In further embodiments, the application relates to a composition comprising a PEM-3-like polypeptide and an E2. An example of an E2 is UBC12.

In certain embodiments, the application provides a fusion protein comprising a PEM-3-like polypeptide and a NEDD8 polypeptide. In certain embodiments, the NEDD8 polypeptide is fused to the N-terminus of the PEM-3-like polypeptide. In other embodiments, the NEDD8 polypeptide is fused to the C-terminus of the PEM-3-like polypeptide. In yet other embodiments, the PEM-3-like fusion protein comprises amino acid sequence selected from the group consisting of: SEQ ID NOS: 50-53.

The application additionally relates to a complex comprising a PEM-3-like polypeptide and a PEM-3-like-AP. In certain embodiments, the PEM-3-like-AP is selected from the group consisting of: an E1, an E2, a PEM-3-like polypeptide, a ubiquitin, and a NEDD8. In certain embodiments, the E2 is selected from the group consisting of: UBCH5, UBC13, and UBC12. In yet other embodiments, the E1 is APP-BP1/Uba3.

In additional embodiments, the application relates to a method of inhibiting viral infection comprising administering an agent to a subject in need thereof wherein said agent inhibits PEM-3-like-protein-mediated viral release. The application further relates to a method of identifying targets for therapeutic intervention comprising identifying a polypeptide that associates with a PEM-3-like polypeptide.

In certain embodiments, the application relates to a method for evaluating the anti-viral potential of a compound comprising: (a) forming a mixture comprising a ubiquitin; an E1; an E2; and a PEM-3-like polypeptide; (b) adding a test agent; and (c) detecting ubiquitin-ligase activity of said PEM-3-like polypeptide, wherein a compound that decreases the ligase activity of said PEM-3-like polypeptide is a potential anti-viral agent. In additional embodiments, the mixture further comprises NEDD8. In certain further embodiments, the PEM-3-like polypeptide is neddylated. In additional embodiments, the PEM-3-like polypeptide is a fusion protein comprising a NEDD8 polypeptide. In certain embodiments, the NEDD8 polypeptide is fused to the N-terminus of the PEM-3-like polypeptide. In other embodiments, the NEDD8 polypeptide is fused to the C-terminus of the PEM-3-like polypeptide. In yet other embodiments, the PEM-3-like fusion protein comprises amino acid sequence selected from the group consisting of: SEQ ID NOS: 50-53.

The application additionally relates to isolated PEM-3-like nucleic acid comprising a nucleic acid sequence at least 85% identical to the nucleic acid sequence depicted in SEQ ID NO: 22. In certain embodiments, the nucleic acid comprises the nucleic acid sequence depicted in SEQ ID NO: 22. In additional embodiments, the application relates to an isolated PEM-3-like polypeptide comprising the amino acid sequence depicted in SEQ ID NO: 23.

In further embodiments, the application relates to an isolated PEM-3-like nucleic acid comprising a nucleic acid sequence at least 85% identical to the nucleic acid sequence depicted in SEQ ID NO: 24. In certain embodiments, the nucleic acid comprises the nucleic acid sequence depicted in SEQ ID NO: 24. In certain further embodiments, the application relates to an isolated PEM-3-like polypeptide comprising the amino acid sequence depicted in SEQ ID NO: 26.

In yet other embodiments, the application relates to an isolated PEM-3-like nucleic acid comprising a nucleic acid sequence at least 85% identical to the nucleic acid sequence depicted in SEQ ID NO: 25. In certain embodiments, the nucleic acid comprises the nucleic acid sequence depicted in SEQ ID NO: 25. In further embodiments, the application relates to an isolated PEM-34-like polypeptide comprising the amino acid sequence depicted in SEQ ID NO: 27.

In some aspects, the invention provides nucleic acid sequences and proteins encoded thereby, methods employing nucleic acid sequences and proteins encoded thereby, as well as oligonucleotides derived from the nucleic acid sequences, antibodies directed to the encoded proteins, screening assays to identify agents that modulate PEM-3-like protein, and diagnostic methods for detecting cells infected with a virus, preferably an enveloped virus, RNA virus and particularly a retrovirus.

In one aspect, the invention provides an isolated nucleic acid comprising a nucleotide sequence which hybridizes under stringent conditions to a sequence of SEQ ID NOs: 22, 24 and/or 25 or a sequence complementary thereto. In another aspect, the invention provides methods employing an isolated nucleic acid comprising a nucleotide sequence which hybridizes under stringent conditions to a sequence of SEQ ID NOS: 1, 3, 5, 7, 9, 11, 13, 15, 17, 19, 22, 24 and/or 25 or a sequence complementary thereto. In a related embodiment, the nucleic acid is at least about 80%, 90%, 95%, or 97-98%, or 100% identical to a sequence corresponding to at least about 12, at least about 15, at least about 25, at least about 40, at least about 100, at least about 300, or at least about 500 consecutive nucleotides up to the full length of SEQ ID NOS: 1, 3, 5, 7, 9, 11, 13, 15, 17, 19, 22, 24 and/or 25 , or a sequence complementary thereto.

In other embodiments, the invention provides a nucleic acid comprising a nucleotide sequence which hybridizes under stringent conditions to a sequence of SEQ ID NOS: 22, 24 and/or 25, or a nucleotide sequence that is at least about 80%, 90%, 95%, or 97-98%, or 100% identical to a sequence corresponding to at least about 12, at least about 15, at least about 25, at least about 40, at least about 100, at least about 300, or at least about 500 consecutive nucleotides up to the full length of SEQ ID NOS: 22, 24 and/or 25, or a sequence complementary thereto, and a transcriptional regulatory sequence operably linked to the nucleotide sequence to render the nucleotide sequence suitable for use as an expression vector. In another embodiment, the nucleic acid may be included in an expression vector capable of replicating in a prokaryotic or eukaryotic cell. In a related embodiment, the invention provides a host cell transfected with the expression vector.

In other embodiments, the invention provides methods employing a nucleic acid comprising a nucleotide sequence which hybridizes under stringent conditions to a sequence of SEQ ID NOS: 1, 3, 5, 7, 9, 11, 13, 15, 17, 19, 22, 24 and/or 25, or a nucleotide sequence that is at least about 80%, 90%, 95%, or 97-98%, or 100% identical to a sequence corresponding to at least about 12, at least about 15, at least about 25, at least about 40, at least about 100, at least about 300, or at least about 500 consecutive nucleotides up to the full length of SEQ ID NOS: 1, 3, 5, 7, 9, 11, 13, 15, 17, 19, 22, 24 and/or 25, or a sequence complementary thereto, and a transcriptional regulatory sequence operably linked to the nucleotide sequence to render the nucleotide sequence suitable for use as an expression vector. In another embodiment, the nucleic acid may be included in an expression vector capable of replicating in a prokaryotic or eukaryotic cell. In a related embodiment, the invention provides a host cell transfected with the expression vector.

In yet another embodiment, the invention provides a substantially pure nucleic acid which hybridizes under stringent conditions to a nucleic acid probe corresponding to at least about 12, at least about 15, at least about 25, or at least about 40 consecutive nucleotides up to the full length of SEQ ID NOS: 22, 24 and/or 25, or a sequence complementary thereto or up to the full length of the gene of which said sequence is a fragment. The invention also provides an antisense oligonucleotide analog which hybridizes under stringent conditions to at least 12, at least 25, or at least 50 consecutive nucleotides up to the full length of SEQ ID NOS: 22, 24 and/or 25, or a sequence complementary thereto.

In yet another embodiment, the invention provides methods employing a substantially pure nucleic acid which hybridizes under stringent conditions to a nucleic acid probe corresponding to at least about 12, at least about 15, at least about 25, or at least about 40 consecutive nucleotides up to the full length of SEQ ID NOS: 1, 3, 5, 7, 9, 11, 13, 15, 17, 19, 22, 24 and/or 25, or a sequence complementary thereto or up to the full length of the gene of which said sequence is a fragment. The invention also provides an antisense oligonucleotide analog which hybridizes under stringent conditions to at least 12, at least 25, or at least 50 consecutive nucleotides up to the full length of SEQ ID NOS: 1, 3, 5, 7, 9, 11, 13, 15, 17, 19, 22, 24 and/or 25, or a sequence complementary thereto.

In a further embodiment, the invention provides a nucleic acid comprising a nucleic acid encoding an amino acid sequence as set forth in any of SEQ ID NOS: 23, 26 or 27 or a nucleic acid complement thereof. In a related embodiment, the invention provides methods employing a nucleic acid comprising a nucleic acid encoding an amino acid sequence as set forth in any of SEQ ID NOS: 2, 4, 6, 8, 10, 12, 14, 16, 18, 20, 23, 26 or 27 or a nucleic acid complement thereof. In a related embodiment, the encoded amino acid sequence is at least about 80%, 90%, 95%, or 97-98%, or 100% identical to a sequence corresponding to at least about 12, at least about 15, at least about 25, or at least about 40, or at least about 100 consecutive amino acids up to the full length of any of SEQ ID NOS: 2, 4, 6, 8, 10, 12, 14, 16, 18, 20, 23, 26 or 27.

In another embodiment, the invention provides a probe/primer comprising a substantially purified oligonucleotide, said oligonucleotide containing a region of nucleotide sequence which hybridizes under stringent conditions to at least about 12, at least about 15, at least about 25, or at least about 40 consecutive nucleotides of sense or antisense sequence selected from SEQ ID NOS: 22, 24 and/or 25. In another embodiment, the invention provides methods employing a probe/primer comprising a substantially purified oligonucleotide, said oligonucleotide containing a region of nucleotide sequence which hybridizes under stringent conditions to at least about 12, at least about 15, at least about 25, or at least about 40 consecutive nucleotides of sense or antisense sequence selected from SEQ ID NOS: 1, 3, 5, 7, 9, 11, 13, 15, 17, 19, 22, 24 and/or 25, or a sequence complementary thereto. In preferred embodiments, the probe selectively hybridizes with a target nucleic acid. In another embodiment, the probe may include a label group attached thereto and able to be detected. The label group may be selected from radioisotopes, fluorescent compounds, enzymes, and enzyme co-factors. The invention further provides arrays of at least about 10, at least about 25, at least about 50, or at least about 100 different probes as described above attached to a solid support.

In another aspect, the invention provides PEM-3-like polypeptides. In one embodiment, the invention pertains to the use of a polypeptide including an amino acid sequence encoded by a nucleic acid comprising a nucleotide sequence which hybridizes under stringent conditions to a sequence of SEQ ID NOS: 22, 24 and/or 25, or a sequence complementary thereto, or a fragment comprising at least about 25, or at least about 40 amino acids thereof

In another aspect, the invention provides methods employing PEM-3-like polypeptides. In one embodiment, the invention pertains to the use of a polypeptide including an amino acid sequence encoded by a nucleic acid comprising a nucleotide sequence which hybridizes under stringent conditions to a sequence of SEQ ID NOS: 1, 3, 5, 7, 9, 11, 13, 15, 17, 19, 22, 24 and/or 25, or a sequence complementary thereto, or a fragment comprising at least about 25, or at least about 40 amino acids thereof.

In a preferred embodiment, the invention relates to a PEM-3-like polypeptide that comprises a sequence that is identical with or homologous to any of SEQ ID NOS: 23, 26 or 27. For instance, a PEM-3-like polypeptide preferably has an amino acid sequence at least 60% homologous to a polypeptide represented by any of SEQ ID NOS: 23, 26 or 27 and polypeptides with higher sequence homologies of, for example, 80%, 90% or 95% are also contemplated. The PEM-3-like polypeptide can comprise a full length protein, such as represented in the sequence listings, or it can comprise a fragment of, for instance, at least 5, 10, 20, 50, 100, 150, 200, 250, 300, 400 or 500 or more amino acids in length.

In a preferred embodiment, the invention relates to methods employing a PEM-3-like polypeptide that comprises a sequence that is identical with or homologous to any of SEQ ID NOS: 2, 4, 6, 8, 10, 12, 14, 16, 18, 20, 23, 26 or 27. For instance, a PEM-3-like polypeptide preferably has an amino acid sequence at least 60% homologous to a polypeptide represented by any of SEQ ID NOS: 2, 4, 6, 8, 10, 12, 14, 16, 18, 20, 23, 26 or 27 and polypeptides with higher sequence homologies of, for example, 80%, 90% or 95% are also contemplated. The PEM-3-like polypeptide can comprise a full length protein, such as represented in the sequence listings, or it can comprise a fragment of, for instance, at least 5, 10, 20, 50, 100, 150, 200, 250, 300, 400 or 500 or more amino acids in length.

In another preferred embodiment, the invention features the use of a purified or recombinant polypeptide fragment of a PEM-3-like polypeptide, which polypeptide has the ability to modulate, e.g., mimic or antagonize, an activity of a wild-type PEM-3-like protein. Preferably, the polypeptide fragment comprises a sequence identical or homologous to an amino acid sequence designated in any of SEQ ID NOS: 2, 4, 6, 8, 10, 12, 14, 16, 18, 20, 23, 26 or 27.

In certain embodiments, the invention relates to methods that employ PEM-3-LIKE polypeptides that can be either an agonist (e.g., mimics), or alternatively, an antagonist of a biological activity of a naturally occurring form of the protein, e.g., the polypeptide is able to modulate the intrinsic biological activity of a PEM-3-like protein or a PEM-3-like protein complex, such as an enzymatic activity, binding to other cellular components, cellular compartmentalization, membrane reorganization and the like.

The subject methods can employ proteins that can also be provided as chimeric molecules, such as in the form of fusion proteins. For instance, the PEM-3-like polypeptide can be provided as a recombinant fusion protein which includes a second polypeptide portion, e.g., a second polypeptide having an amino acid sequence unrelated (heterologous) to PEM-3-like protein, e.g., the second polypeptide portion is NEDD8, e.g., the second polypeptide portion is glutathione-S-transferase, e.g., the second polypeptide portion is an enzymatic activity such as alkaline phosphatase, e.g., the second polypeptide portion is an epitope tag, etc.

Yet another aspect of the present invention concerns the use of an immunogen comprising a PEM-3-like polypeptide in an immunogenic preparation, the immunogen being capable of eliciting an immune response specific for the PEM-3-like polypeptide; e.g., a humoral response, e.g., an antibody response; e.g., a cellular response. In preferred embodiments, the immunogen comprises an antigenic determinant, e.g., a unique determinant, from a protein represented by SEQ ID NOS: 2, 4, 6, 8, 10, 12, 14, 16, 18, 20, 23, 26 or 27.

In yet another aspect, this invention provides antibodies immunoreactive with one or more PEM-3-like polypeptides. In one embodiment, antibodies are specific for a KH domain or a RING domain derived from a PEM-3-like polypeptide. In a more specific embodiment, the domain is part of an amino acid sequence set forth in SEQ ID NOS: 2, 4, 6, 8, 10, 12, 14, 16, 18, 20, 23, 26 or 27. In a set of exemplary embodiments, an antibody binds to one or more KH domains. In another exemplary embodiment, an antibody binds to a RING domain. In another embodiment, the antibodies are immunoreactive with one or more proteins having an amino acid sequence that is at least 80% identical, at least 90% identical or at least 95% identical to an amino acid sequence as set forth in SEQ ID NOS: 2, 4, 6, 8, 10, 12, 14, 16, 18, 20, 23, 26 or 27. In other embodiments, an antibody is immunoreactive with one or more proteins having an amino acid sequence that is 85%, 90%, 95%, 98%, 99% or identical to an amino acid sequence as set forth in SEQ ID NOS: 2, 4, 6, 8, 10, 12, 14, 16, 18, 20,23, 26 or 27.

In certain embodiments, the invention relates to methods that employ PEM-3-like nucleic acids that include a transcriptional regulatory sequence, e.g., at least one of a transcriptional promoter or transcriptional enhancer sequence, which regulatory sequence is operably linked to the PEM-3-like sequence. Such regulatory sequences can be used to render the PEM-3-like sequence suitable for use as an expression vector.

In certain embodiments, the invention relates to methods to identify an antiviral agent. In certain aspects, the invention relates to a method to identify an antiviral agent wherein the agent is identified by its ability to interact with and/or modulate an activity of a PEM-3-like polypeptide.

In yet another aspect, the invention provides an assay for screening test compounds for inhibitors, or alternatively, potentiators, of an interaction between a PEM-3-like polypeptide and a PEM-3-like-polypeptide-associated protein (PEM-3-like-AP) such as a late domain region of an RNA virus such as a retrovirus. An exemplary method includes the steps of (i) combining PEM-3-like-AP, a PEM-3-like polypeptide, and a test compound, e.g., under conditions wherein, but for the test compound, the PEM-3-like polypeptide and PEM-3-like-AP are able to interact; and (ii) detecting the formation of a complex which includes the PEM-3-like polypeptide and a PEM-3-like-AP. A statistically significant change, such as a decrease, in the formation of the complex in the presence of a test compound (relative to what is seen in the absence of the test compound) is indicative of a modulation, e.g., inhibition, of the interaction between the PEM-3-like polypeptide and PEM-3-like-AP.

In a further embodiment, the invention provides an assay for identifying a test compound which inhibits or potentiates the interaction of a PEM-3-like polypeptide to a PEM-3-like-AP, comprising (a) forming a reaction mixture including PEM-3-like polypeptide, a PEM-3-like-AP; and a test compound; and detecting binding of said PEM-3-like polypeptide to said PEM-3-like-AP; wherein a change in the binding of said PEM-3-like polypeptide to said PEM-3-like-AP in the presence of the test compound, relative to binding in the absence of the test compound, indicates that said test compound potentiates or inhibits binding of said PEM-3-like polypeptide to said PEM-3-like-AP.

In an additional embodiment, the invention relates to a method for identifying modulators of protein complexes, comprising (a) forming a reaction mixture comprising a PEM-3-like polypeptide, a PEM-3-like-AP; and a test compound; (b) contacting the reaction mixture with a test agent, and (c) determining the effect of the test agent for one or more activities. Exemplary activities include a change in the level of the protein complex, a change in the enzymatic activity of the complex, where the reaction mixture is a whole cell, a change in the plasma membrane localization of the complex or a component thereof or a change in the interaction between the PEM-3-like polypeptide and the PEM-3-like-AP.

An additional embodiment is a screening assay to identify agents that inhibit or potentiate the interaction of a PEM-3-like polyp eptide and a PEM-3-like-AP, comprising providing a two-hybrid assay system including a first fusion protein comprising a PEM-3-like polypeptide portion of SEQ ID NOS: 2, 4, 6, 8, 10, 12, 14, 16, 18, 20, 23, 26 or 27, and a second fusion protein comprising a PEM-3-like-AP portion, under conditions wherein said two hybrid assay is sensitive to interactions between the PEM-3-like polypeptide portion of said first fusion protein and said PEM-3-like-AP portion of said second polypeptide; measuring a level of interactions between said fusion proteins in the presence and in the absence of a test agent; and comparing the level of interaction of said fusion proteins, wherein a decrease in the level of interaction is indicative of an agent that will inhibit the interaction between a PEM-3-like polypeptide and a PEM-3-like-AP.

In additional aspects, the invention provides isolated protein complexes including a combination of a PEM-3-like polypeptide and at least one PEM-3-like-AP. In certain embodiments, a PEM-3-like complex is related to clathrin-coated vesicle formation. In a further embodiment, a PEM-3-like complex comprises a viral protein, such as Gag.

In an additional aspect, the invention provides nucleic acid therapies for manipulating PEM-3-like polypeptides. In one embodiment, the invention provides a method employing a ribonucleic acid comprising between 5 and 500 consecutive nucleotides of a nucleic acid sequence that is at least 90%, 95%, 98%, 99% or optionally 100% identical to a sequence of SEQ ID NOS: 1, 3, 5, 7, 9, 11, 13, 15, 17, 19, 22, 24 and/or 25 or a complement thereof. Optionally the ribonucleic acid comprises at least 10, 15, 20, 25, or 30 consecutive nucleotides, and no more than 1000, 750, 500 and 250 consecutive nucleotides of a PEM-3-like nucleic acid. In certain embodiments the ribonucleic acid is an RNAi oligomer or a ribozyme. Preferably, the ribonucleic acid decreases the level of a PEM-3-like mRNA.

The invention also features transgenic non-human animals, e.g., mice, rats, rabbits, goats, sheep, dogs, cats, cows, or non-human primates, having a transgene, e.g., animals which include (and preferably express) a heterologous form of the PEM-3-like gene described herein. Such a transgenic animal can serve as an animal model for studying viral infections such as HIV infection or for use in drug screening for viral infections.

In further aspects, the invention provides compositions for the delivery of a nucleic acid therapy, such as, for example, compositions comprising a liposome and/or a pharmaceutically acceptable excipient or carrier.

The practice of the present invention will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Molecular Cloning A Laboratory Manual, 2nd Ed., ed. by Sambrook, Fritsch and Maniatis (Cold Spring Harbor Laboratory Press: 1989); DNA Cloning, Volumes I and II (D. N. Glover ed., 1985); Oligonucleotide Synthesis (M. J. Gait ed., 1984); Mullis et al. U.S. Pat. No. 4,683,195; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells

(J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Manipulating the Mouse Embryo, (Cold Spring Harbor Laboratory Press, Cold Spring Harbor, N.Y., 1986).

Other features and advantages of the invention will be apparent from the following detailed description, and from the claims.

### BRIEF DESCRIPTION OF THE DRAWINGS

FIG. 1: Human PEM-3-like protein mRNA sequence public gi: 21755617; SEQ ID NO: 1)

FIG. 2: Human PEM-3-like protein amino acid sequence encoded by SEQ ID NO: 1 (SEQ ID NO: 2)

FIG. 3: Human PEM-3-like protein mRNA sequence (public gi: 21734163; SEQ ID NO: 3)

FIG. 4: Human PEM-3-like protein amino acid sequence encoded by SEQ ID NO: 3 (SEQ ID NO: 4)

FIG. 5: Human PEM-3-like protein mRNA sequence (public gi: 21438819; SEQ ID NO: 5)

FIG. 6: Human PEM-3-like protein amino acid sequence (public gi: 21438820; SEQ ID NO: 6)

FIG. 7: Human PEM-3-like protein mRNA sequence (public gi: 7706165; SEQ ID NO: 7)

FIG. 8: Human PEM-3-like protein amino acid sequence (public gi: 7706166; SEQ ID NO: 8)

FIG. 9: Human PEM-3-like protein mRNA sequence (public gi: 7582297; SEQ ID NO: 9)

FIG. 10: Human PEM-3-like protein amino acid sequence (public gi: 7582298; SEQ ID NO: 10)

FIG. 11: Human PEM-3-like protein mRNA sequence (public gi: 27370677; SEQ ID NO: 11)

FIG. 12: Human PEM-3-like protein amino acid sequence encoded by SEQ ID NO: 11 (SEQ ID NO: 12)

FIG. 13: Human PEM-3-like protein mRNA sequence (public gi: 21432052; SEQ ID NO: 13)

FIG. 14: Human PEM-3-like protein amino acid sequence encoded by SEQ ID NO: 13 (SEQ ID NO: 14)

FIG. 15: Human PEM-3-like protein mRNA sequence (public gi: 15250817; SEQ ID NO: 15)

FIG. 16: Human PEM-3-like protein amino acid sequence encoded by SEQ ID NO: 15 (SEQ ID NO: 16)

FIG. 17: Human PEM-3-like protein mRNA sequence (public gi: 15250983; SEQ ID NO: 17)

FIG. 18: Human PEM-3-like protein amino acid sequence encoded by SEQ ID NO: 17 (SEQ ID NO: 18)

FIG. 19: Human PEM-3-like protein mRNA sequence (public gi: 15345043; SEQ ID NO: 19)

FIG. 20: Human PEM-3-like protein amino acid sequence encoded by SEQ ID NO: 19 (SEQ ID NO: 20)

FIG. 21: Sequence analysis of PEM-3-like protein.

FIG. 22: Protein sequence alignment of the different alternative splicing of human PEM-3-like protein.

FIG. 23: Protein domains and motifs of SEQ ID NO: 2.

FIG. 24: Protein domains and motifs of SEQ ID NO: 4.

FIG. 25: Protein domains and motifs of SEQ ID NO: 6.

FIG. 26: Protein domains and motifs of SEQ ID NO: 8.

FIG. 27: Protein domains and motifs of SEQ ID NO: 10.

FIG. 28: Protein domains and motifs of SEQ ID NO: 12.

FIG. 29: Protein domains and motifs of SEQ ID NO: 14.

FIG. 30: Protein domains and motifs of SEQ ID NO: 16.

FIG. 31: Protein domains and motifs of SEQ ID NO: 18.

FIG. 32: Protein domains and motifs of SEQ ID NO: 20.

FIG. 33: PEM-3-like protein affects the release of virus-like particles (“VLP”) from cells at steady state. A) Western Blot analysis of VLP release from cells. B) Quantification of viral budding.

FIG. 34: Human PEM-3-like protein mRNA sequence (SEQ ID NO: 22).

FIG. 35: Human PEM-3-like protein amino acid sequence encoded by SEQ ID NO: 22 (SEQ ID NO: 23).

FIG. 36: Domain analysis of PEM-3-LIKE protein (SEQ ID NO: 23).

FIG. 37: Human PEM-3-like protein mRNA sequence (SEQ ID NO: 24).

FIG. 38: Human PEM-3-like protein mRNA sequence (SEQ ID NO: 25).

FIG. 39: Human PEM-3-like protein amino acid sequence encoded by SEQ ID NO: 24 (SEQ ID NO: 26).

FIG. 40: Human PEM-3-like protein amino acid sequence encoded by SEQ ID NO: 25 (SEQ ID NO: 27).

FIG. 41: Domain analysis of PEM-3-LIKE protein (SEQ ID NO: 26).

FIG. 42: Reverse transcriptase (“RT”) activity in VLP secreted from cells treated with indicated siRNAs. HeLa SS6 cell cultures (in triplicates) were transfected with siRNA targeting PEM-3-like protein or with a control siRNA. Following gene silencing by siRNA, cells were transfected with pNLenvl, encoding an envelope-deficient subviral Gag-Pol expression system and RT activity in VLP released into the culture medium was determined. Cells treated with PEM-3-like-specific siRNA reduced RT activity by 90 percent.

FIG. 43: SEM analysis of cells transfected with pNLenv-1 and control or PEM-3-like RNAi. Scanning electron microscopy (SEM) revealed numerous cell surface-tethered virus particles, consistent with inhibition of virus release. Pre-treatment with PEM-3-like siRNA ablated virus budding, indicating that it functions independently of the virus L-domain and upstream of virus budding at the cell membrane (compare control and PEM-3-like RNAi).

FIG. 44: PEM-3-like is important for HIV-1 infectivity. A. Hela SS6 cells were co-transfected with plasmids encoding HIV-1 (see materials and methods) and RNAi directed against PEM-3-like or control RNAi. Twenty four hours post transfection viruses were collected and used to infect target HEK 293T cells. Percent infection was determined by FACS analysis of GFP-positive cells. B. Hela SS6 cells were co-trasnfected with control or PEM-3-like specific RNAi and a plasmid encoding GFP-PEM-3-like tester plasmid to detect the efficiency of PEM-3-like reduction. The upper panels depict GFP fluorescence and the lower panel phase micrsocopy.

FIG. 45: PEM-3-like is a ubiquitin protein ligase. GST-PEM-3-like was incubated with and E1 and E2 (two different concentrations, UbcH6c or UBC13/Uev1, as indicated above each lane) in a complete ubiquitination reaction. In control reactions, GST-PEM-3-like was omitted. At the end of ubiquitination, twenty-five percent of the reaction was removed and analyzed by SDS-PAGE and immunoblot analysis for the appearance of free ubiquiitn chans (left upper panel) and PEM-3-like (left lower panel). To the rest of the reaction GSH-agarose beads were added to separate GST-PEM-3-like from the reaction and anlyze its ubiquitination and levels by SDS-PAGE and immunoblot analysis (right, upper and lower panels, respectively).

FIG. 46: PEM-3-like is an E3: conjugates ubiquitin to itself and forms free ubiquitin chains with UBC13/Uev1.

FIG. 47: Immunoblot of PEM-3-like protein.

### DETAILED DESCRIPTION OF THE INVENTION

1. Definitions

The term “binding” refers to a direct association between two molecules, due to, for example, covalent, electrostatic, hydrophobic, ionic and/or hydrogen-bond interactions under physiological conditions.

“Cells,” “host cells” or “recombinant host cells” are terms used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A “chimeric protein” or “fusion protein” is a fusion of a first amino acid sequence encoding a polypeptide with a second amino acid sequence defining a domain foreign to and not substantially homologous with any domain of the first amino acid sequence. A chimeric protein may present a foreign domain which is found (albeit in a different protein) in an organism which also expresses the first protein, or it may be an “interspecies”, “intergenic”, etc. fusion of protein structures expressed by different kinds of organisms.

The terms “compound”, “test compound” and “molecule” are used herein interchangeably and are meant to include, but are not limited to, peptides, nucleic acids, carbohydrates, small organic molecules, natural product extract libraries, and any other molecules (including, but not limited to, chemicals, metals and organometallic compounds).

The phrase “conservative amino acid substitution” refers to grouping of amino acids on the basis of certain common properties. A functional way to define common properties between individual amino acids is to analyze the normalized frequencies of amino acid changes between corresponding proteins of homologous organisms (Schulz, G. B. and R. H. Schirmer., Principles of Protein Structure, Springer-Verlag). According to such analyses, groups of amino acids may be defined where amino acids within a group exchange preferentially with each other, and therefore resemble each other most in their impact on the overall protein structure (Schulz, G. E. and R. H. Schirmer, Principles of Protein Structure, Springer-Verlag). Examples of amino acid groups defined in this manner include:
(i) a charged group, consisting of Glu and Asp, Lys, Arg and His,
(ii) a positively-charged group, consisting of Lys, Arg and His,
(iii) a negatively-charged group, consisting of Glu and Asp,
(iv) an aromatic group, consisting of Phe, Tyr and Trp,
(v) a nitrogen ring group, consisting of His and Trp,
(vi) a large aliphatic nonpolar group, consisting of Val, Leu and Ile,
(vii) a slightly-polar group, consisting of Met and Cys,
(viii) a small-residue group, consisting of Ser, Thr, Asp, Asn, Gly, Ala, Glu, Gln and Pro,
(ix) an aliphatic group consisting of Val, Leu, Ile, Met and Cys, and
(x) a small hydroxyl group consisting of Ser and Thr.

In addition to the groups presented above, each amino acid residue may form its own group, and the group formed by an individual amino acid may be referred to simply by the one and/or three letter abbreviation for that amino acid commonly used in the art.

A “conserved residue” is an amino acid that is relatively invariant across a range of similar proteins. Often conserved residues will vary only by being replaced with a similar amino acid, as described above for “conservative amino acid substitution”.

The term “domain” as used herein refers to a region of a protein that comprises a particular structure and/or performs a particular function.

“Homology” or “identity” or “similarity” refers to sequence similarity between two peptides or between two nucleic acid molecules. Homology and identity can each be determined by comparing a position in each sequence which may be aligned for purposes of comparison. When an equivalent position in the compared sequences is occupied by the same base or amino acid, then the molecules are identical at that position; when the equivalent site occupied by the same or a similar amino acid residue (e.g., similar in steric and/or electronic nature), then the molecules can be referred to as homologous (similar) at that position. Expression as a percentage of homology/similarity or identity refers to a function of the number of identical or similar amino acids at positions shared by the compared sequences. A sequence which is “unrelated” or “non-homologous” shares less than 40% identity, though preferably less than 25% identity with a sequence of the present invention. In comparing two sequences, the absence of residues (amino acids or nucleic acids) or presence of extra residues also decreases the identity and homology/similarity.

The term “homology” describes a mathematically based comparison of sequence. similarities which is used to identify genes or proteins with similar functions or motifs. The nucleic acid and protein sequences of the present invention may be used as a “query sequence” to perform a search against public databases to, for example, identify other family members, related sequences or homologs. Such searches can be performed using the NBLAST and XBLAST programs (version 2.0) of Altschul, et al. (1990) J Mol. Biol. 215:403-10. BLAST nucleotide searches can be performed with the NBLAST program, score=100, wordlength=12 to obtain nucleotide sequences homologous to nucleic acid molecules of the invention. BLAST protein searches can be performed with the XBLAST program, score=50, wordlength=3 to obtain amino acid sequences homologous to protein molecules of the invention. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and BLAST) can be used. See http://www.ncbi.nlm.nih.gov.

As used herein, “identity” means the percentage of identical nucleotide or amino acid residues at corresponding positions in two or more sequences when the sequences are aligned to maximize sequence matching, i.e., taking into account gaps and insertions. Identity can be readily calculated by known methods, including but not limited to those described in (Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991; and Carillo, H., and Lipman, D., SLAM J. Applied Math., 48: 1073 (1988). Methods to determine identity are designed to give the largest match between the sequences tested. Moreover, methods to determine identity are codified in publicly available computer programs. Computer program methods to determine identity between two sequences include, but, are not limited to, the GCG program package (Devereux, J., et al., Nucleic Acids Research 12(1): 387 (1984)), BLASTP, BLASTN, and FASTA (Altschul, S. F. et al., J. Molec. Biol. 215: 403-410 (1990) and Altschul et al. Nuc. Acids Res. 25: 3389-3402 (1997)). The BLAST X program is publicly available from NCBI and other sources (BLAST Manual, Altschul, S., et al., NCBI NLM NIH Bethesda, Md. 20894; Altschul, S., et al., J. Mol. Biol. 215: 403-410 (1990). The well known Smith Waterman algorithm may also be used to determine identity.

The term “intron” refers to a portion of nucleic acid that is intially transcribed into RNA but later removed such that it is not, for the most part, represented in the processed mRNA. Intron removal occurs through reactions at the 5′ and 3′ ends, typically referred to as 5′ and 3′ splice sites, respectively. Alternate use of different splice sites results in splice variants. An intron is not necessarily situated between two “exons”, or portions that code for amino acids, but may instead be positioned, for example, between the promoter and the first exon. An intron may be self-splicing or may require cellular components to be spliced out of the mRNA. A “heterologous intron” is an intron that is inserted into a coding sequence that is not naturally associated with that coding sequence. In addition, a heterologous intron may be a generally natural intron wherein one or both of the splice sites have been altered to provide a desired quality, such as increased or descreased splice efficiency. Heterologous introns are often inserted, for example, to improve expression of a gene in a heterologous host, or to increase the production of one splice variant relative to another. As an example, the rabbit beta-globin gene may be used, and is commercially available on the pCI vector from Promega Inc. Other exemplary introns are provided in Lacy-Hulbert et al. (2001) Gene Ther 8(8):649-53.

The term “isolated”, as used herein with reference to the subject proteins and protein complexes, refers to a preparation of protein or protein complex that is essentially free from contaminating proteins that normally would be present with the protein or complex, e.g., in the cellular milieu in which the protein or complex is found endogenously. Thus, an isolated protein complex is isolated from cellular components that normally would “contaminate” or interfere with the study of the complex in isolation, for instance while screening for modulators thereof. It is to be understood, however, that such an “isolated” complex may incorporate other proteins the modulation of which, by the subject protein or protein complex, is being investigated.

The term “isolated” as also used herein with respect to nucleic acids, such as DNA or RNA, refers to molecules in a form which does not occur in nature. Moreover, an “isolated nucleic acid” is meant to include nucleic acid fragments which are not naturally occurring as fragments and would not be found in the natural state.

A “KH domain” or “K homology domain” is a protein domain associated with RNA-binding. The KH domain was first identified as a 45 amino acid repeat in the heterogeneous nuclear ribonucleoprotein K. A KH domain typically contains the consensus RNA-binding motif represented by VIGXXGXXI.

Lentiviruses include primate lentiviruses, e.g., human immunodeficiency virus types 1 and 2 (HIV-1/HIV-2); simian immunodeficiency virus (SIV) from Chiimpanzee (SIVcpz), Sooty mangabey (SIVsmm), African Green Monkey (SIVagm), Syke's monkey (SIVsyk), Mandrill (SIVmnd) and Macaque (SIVmac). Lentiviruses also include feline lentiviruses, e.g., Feline immunodeficiency virus (FIV); Bovine lentiviruses, e.g., Bovine immunodeficiency virus (BIV); Ovine lentiviruses, e.g., Maedi/Visna virus (MVV) and Caprine arthritis encephalitis virus (CAEV); and Equine lentiviruses, e.g., Equine infectious anemia virus (EIAV). All lentiviruses express at least two additional regulatory proteins (Tat, Rev) in addition to Gag, Pol, and Env proteins. Primate lentiviruses produce other accessory proteins including Nef, Vpr, Vpu, Vpx, and Vif. Generally, lentiviruses are the causative agents of a variety of disease, including, in addition to immunodeficiency, neurological degeneration, and arthritis. Nucleotide sequences of the various lentiviruses can be found in Genbank under the following Accession Nos. (from J. M. Coffin, S. H. Hughes, and H. E. Varmus, “Retroviruses” Cold Spring Harbor Laboratory Press, 199,7 p 804): 1) HIV-1: K03455, M19921, K02013, M3843 1, M38429, K02007 and M17449; 2) HIV-2: M30502, J04542, M30895, J04498, M15390, M31113 and L07625; 3) SIV:M29975, M30931, M58410, M66437, L06042, M33262, M19499, M32741, M31345 and L03295; 4) FIV: M25381, M36968 and UI 1820; 5)BIV. M32690; 6)E1AV: M16575, M87581 and U01866; 6)Visna: M10608, M51543, L06906, M60609 and M60610; 7) CAEV: M33677; and 8) Ovine lentivirus M31646 and M34193. Lentiviral DNA can also be obtained from the American Type Culture Collection (ATCC). For example, feline immunodeficiency virus is available under ATCC Designation No. VR-2333 and VR-3112. Equine infectious anemia virus A is available under ATCC Designation No. VR-778. Caprine arthritis-encephalitis virus is available under ATCC Designation No. VR-905. Visna virus is available under ATCC Designation No. VR-779. As used herein, the term “nucleic acid” refers to polynucleotides such as deoxyribonucleic acid (DNA), and, where appropriate, ribonucleic acid (RNA). The term should also be understood to include, as equivalents, analogs of either RNA or DNA made from nucleotide analogs, and, as applicable to the embodiment being described, single-stranded (such as sense or antisense) and double-stranded polynucleotides.

The term “maturation” as used herein refers to the production, post-translational processing, assembly and/or release of proteins that form a viral particle. Accordingly, this includes the processing of viral proteins leading to the pinching off of nascent virion from the cell membrane.

A “membrane associated protein” is meant to include proteins that are integral membrane proteins as well as proteins that are stably associated with a membrane.

The term “p6” or p6gag” is used herein to refer to a protein comprising a viral L domain. Antibodies that bind to a p6 domain are referred to as “anti-p6 antibodies”. p6 also refers to proteins that comprise artificially engineered L domains including, for example, L domains comprising a series of L motifs. The term “Gag protein” or “Gag polypeptide” refers to a polypeptide having Gag activity and preferably comprising an L (or late) domain. Exemplary Gag proteins include a motif such as PXXP, PPXY, RXXPXXP, RPDPTAP, RPLPVAP, RPEPTAP, YEDL, PTAPPEY and/or RPEPTAPPEE. HIV p24 is an exemplary Gag polypeptide.

A “PEM-3-like nucleic acid” is a nucleic acid comprising a sequence as represented in any of SEQ ID NOS: 1, 3, 5, 7, 9, 11, 13, 15, 17, 19, 22, 24 and 25 as well as any of the variants described herein.

A “PEM-3-like polypeptide” or “PEM-3-like protein” is a polypeptide comprising a sequence as represented in any of SEQ ID NOS: 2, 4, 6, 8, 10, 12, 14, 16, 18, 20, 23, 26 and 27 as well as any of the variations described herein.

A “PEM-3-like-polypeptide-associated protein” or “PEM-3-like-AP” refers to a protein capable of interacting with and/or binding to a PEM-3-like polypeptide. Generally, the PEM-3-like-AP may interact directly or indirectly with the PEM-3-like polypeptide.

A “profile” is used herein to indicate an aggregate of information regarding a preparation of cell or membrane surface proteins. A profile will comprise, at minimum, information regarding the presence or absence of such proteins. More typically, a profile will comprise information regarding the presence or absence of a plurality of such proteins. In addition, a profile may contain other information about each identified protein, such as relative or absolute amount of protein present, the degree of post-translational modification, membrane topology, three-dimensional structure, isoelectric point, molecular weight, etc. A “test profile” is a profile obtained from a subject of unknown diagnostic state. A “reference profile” is a profile obtained from subject known to be infected or uninfected.

The terms peptides, proteins and polypeptides are used interchangeably herein.

The term “purified protein” refers to a preparation of a protein or proteins which are preferably isolated from, or otherwise substantially free of, other proteins normally associated with the protein(s) in a cell or cell lysate. The term “substantially free of other cellular proteins” (also referred to herein as “substantially free of other contaminating proteins”) is defined as encompassing individual preparations of each of the component proteins comprising less than 20% (by dry weight) contaminating protein, and preferably comprises less than 5% contaminating protein. Functional forms of each of the component proteins can be prepared as purified preparations by using a cloned gene as described in the attached examples. By “purified”, it is meant, when referring to component protein preparations used to generate a reconstituted protein mixture, that the indicated molecule is present in the substantial absence of other biological macromolecules, such as other proteins (particularly other proteins which may substantially mask, diminish, confuse or alter the characteristics of the component proteins either as purified preparations or in their function in the subject reconstituted mixture). The term “purified” as used herein preferably means at least 80% by dry weight, more preferably in the range of 85% by weight, more preferably 95-99% by weight, and most preferably at least 99.8% by weight, of biological macromolecules of the same type present (but water, buffers, and other small molecules, especially molecules having a molecular weight of less than 5000, can be present). The term “pure” as used herein preferably has the same numerical limits as “purified” immediately above.

A “receptor” or “protein having a receptor function” is a protein that interacts with an extracellular ligand or a ligand that is within the cell but in a space that is topologically equivalent to the extracellular space (e.g., inside the Golgi, inside the endoplasmic reticulum, inside the nuclear membrane, inside a lysosome or transport vesicle, etc.). Exemplary receptors are identified herein by annotation as such in various public databases. Receptors often have membrane domains.

A “recombinant nucleic acid” is any nucleic acid that has been placed adjacent to another nucleic acid by recombinant DNA techniques. A “recombined nucleic acid” also includes any nucleic acid that has been placed next to a second nucleic acid by a laboratory genetic technique such as, for example, tranformation and integration, transposon hopping or viral insertion. In general, a recombined nucleic acid is not naturally located adjacent to the second nucleic acid.

The term “recombinant protein” refers to a protein of the present invention which is produced by recombinant DNA techniques, wherein generally DNA encoding the expressed protein is inserted into a suitable expression vector which is in turn used to transform a host cell to produce the heterologous protein. Moreover, the phrase “derived from”, with respect to a recombinant gene encoding the recombinant protein is meant to include within the meaning of “recombinant protein” those proteins having an amino acid sequence of a native protein, or an amino acid sequence similar thereto which is generated by mutations including substitutions and deletions of a naturally occurring protein.

A “RING domain” or “Ring Finger” is a zinc-binding domain with a defined octet of cysteine and histidine residues. Certain RING domains comprise the consensus sequences as set forth below (amino acid nomenclature is as set forth in Table 1): Cys Xaa Xaa Cys Xaa₁₀₋₂₀ Cys Xaa His Xaa₂₋₅ Cys Xaa Xaa Cys Xaa₁₃₋₅₀ Cys Xaa Xaa Cys or Cys Xaa Xaa Cys Xaa₁₀₋₂₀ Cys Xaa His Xaa₂₋₅ His Xaa Xaa Cys Xaa₁₃₋₅₀ Cys Xaa Xaa Cys. Preferred RING domains of the invention bind to various protein partners to form a complex that has ubiquitin ligase activity. RING domains preferably interact with at least one of the following protein types: F box proteins, E2 ubiquitin conjugating enzymes and cullins.

The term “RNA interference” or “RNAi” refers to any method by which expression of a gene or gene product is decreased by introducing into a target cell one or more double-stranded RNAs which are homologous to the gene of interest (particularly to the messenger RNA of the gene of interest). RNAi may also be achieved by introduction of a DNA:RNA hybrid wherein the antisense strand (relative to the target) is RNA. Either strand may include one or more modifications to the base or sugar-phosphate backbone. Any nucleic acid preparation designed to achieve an RNA interference effect is referred to herein as an “RNAi construct”. RNAi constructs include small interfering RNAs (siRNAs), hairpin RNAs, and other RNA species which can be cleaved in vivo to form siRNAs.

“Small molecule” as used herein, is meant to refer to a composition, which has a molecular weight of less than about 5 kD and most preferably less than about 2.5 kD. Small molecules can be nucleic acids, peptides, polypeptides, peptidomimetics, carbohydrates, lipids or other organic (carbon containing) or inorganic molecules. Many pharmaceutical companies have extensive libraries of chemical and/or biological mixtures comprising arrays of small molecules, often fungal, bacterial, or algal extracts, which can be screened with any of the assays of the invention.

As used herein, the term “specifically hybridizes” refers to the ability of a nucleic acid probe/primer of the invention to hybridize to at least 12, 15, 20, 25, 30, 35, 40, 45, 50 or 100 consecutive nucleotides of a PEM-3-like sequence, or a sequence complementary thereto, or naturally occurring mutants thereof, such that it has less than 15%, preferably less than 10%, and more preferably less than 5% background hybridization to a cellular nucleic acid (e.g., mRNA or genomic DNA) other than the PEM-3-like gene. A variety of hybridization conditions may be used to detect specific hybridization, and the stringency is determined primarily by the wash stage of the hybridization assay. Generally high temperatures and low salt concentrations give high stringency, while low temperatures and high salt concentrations give low stringency. Low stringency hybridization is achieved by washing in, for example, about 2.0×SSC at 50° C., and high stringency is acheived with about 0.2×SSC at 50° C. Further descriptions of stringency are provided below.

As applied to polypeptides, “substantial sequence identity” means that two peptide sequences, when optimally aligned, such as by the programs GAP or BESTFIT using default gap which share at least 90 percent sequence identity, preferably at least 95 percent sequence identity, more preferably at least 99 percent sequence identity or more. Preferably, residue positions which are not identical differ by conservative amino acid substitutions. For example, the substitution of amino acids having similar chemical properties such as charge or polarity are not likely to effect the properties of a protein. Examples include glutamine for asparagine or glutamic acid for aspartic acid.

“Transcriptional regulatory sequence” is a generic term used throughout the specification to refer to DNA sequences, such as initiation signals, enhancers, and promoters, which induce or control transcription of protein coding sequences with which they are operably linked. In preferred embodiments, transcription of a recombinant protein gene is under the control of a promoter sequence (or other transcriptional regulatory sequence) which controls the expression of the recombinant gene in a cell-type in which expression is intended. It will also be understood that the recombinant gene can be under the control of transcriptional regulatory sequences which are the same or which are different from those sequences which control transcription of the naturally-occurring form of the protein.

As used herein, a “transgenic animal” is any animal, preferably a non-human mammal, bird or an amphibian, in which one or more of the cells of the animal contain heterologous nucleic acid introduced by way of human intervention, such as by transgenic techniques well known in the art. The nucleic acid is introduced into the cell, directly or indirectly by introduction into a precursor of the cell, by way of deliberate genetic manipulation, such as by microinjection or by infection with a recombinant virus. The term genetic manipulation does not include classical cross-breeding, or in vitro fertilization, but rather is directed to the introduction of a recombinant DNA molecule. This molecule may be integrated within a chromosome, or it may be extrachromosomally replicating DNA. In the typical transgenic animals described herein, the transgene causes cells to express a recombinant human PEM-3-like protein. The “non-human animals” of the invention include vertebrates such as rodents, non-human primates, sheep, dog, cow, chickens, amphibians, reptiles, etc. Preferred non-human animals are selected from the rodent family including rat and mouse, most preferably mouse, though transgenic amphibians, such as members of the Xenopus genus, and transgenic chickens can also provide important tools for understanding and identifying agents which can affect, for example, embryogenesis and tissue formation. The term “chimeric animal” is used herein to refer to animals in which the recombinant gene is found, or in which the recombinant is expressed in some but not all cells of the animal. The term “tissue specific chimeric animal” indicates that the recombinant human PEM-3-like gene is present and/or expressed in some tissues but not others. As used herein, the term “transgene” means a nucleic acid sequence (encoding, e.g., human PEM-3-like polypeptides), which is partly or entirely heterologous, i.e., foreign, to the transgenic animal or cell into which it is introduced, or, is homologous to an endogenous gene of the transgenic animal or cell into which it is introduced, but which is designed to be inserted, or is inserted, into the animal's genome in such a way as to alter the genome of the cell into which it is inserted (e.g., it is inserted at a location which differs from that of the natural gene or its insertion results in a knockout). A transgene can include one or more transcriptional regulatory sequences and any other nucleic acid, such as introns, that may be necessary for optimal expression of a selected nucleic acid.

As is well known, genes for a particular polypeptide may exist in single or multiple copies within the genome of an individual. Such duplicate genes may be identical or may have certain modifications, including nucleotide substitutions, additions or deletions, which all still code for polypeptides having substantially the same activity.

As used herein, the term “vector” refers to a nucleic acid molecule capable of transporting another nucleic acid to which it has been linked. One type of preferred vector is an episome, i.e., a nucleic acid capable of extra-chromosomal replication. Preferred vectors are those capable of autonomous replication and/expression of nucleic acids to which they are linked. Vectors capable of directing the expression of genes to which they are operatively linked are referred to herein as “expression vectors”. In general, expression vectors of utility in recombinant DNA techniques are often in the form of “plasmids” which refer to circular double stranded DNA loops which, in their vector form are not bound to the chromosome. In the present specification, “plasmid” and “vector” are used interchangeably as the plasmid is the most commonly used form of vector. However, the invention is intended to include such other forms of expression vectors which serve equivalent functions and which become known in the art subsequently hereto.

A “virion” is a complete viral particle; nucleic acid and capsid (and a lipid envelope in some viruses.

TABLE 1
Abbreviations for classes of amino acids*

| Symbol | Category | Amino Acids Represented |
|---|---|---|
| X1 | Alcohol | Ser, Thr |
| X2 | Aliphatic | Ile, Leu, Val |
| Xaa | Any | Ala, Cys, Asp, Glu, Phe, |
| | | Gly, His, Ile, Lys, Leu, Met, |
| | | Asn, Pro, Gln, Arg, Ser, |
| | | Thr, Val, Trp, Tyr |
| X4 | Aromatic | Phe, His, Trp, Tyr |
| X5 | Charged | Asp, Glu, His, Lys, Arg |
| X6 | Hydrophobic | Ala, Cys, Phe, Gly, His, Ile, |
| | | Lys, Leu, Met, Thr, Val, |
| | | Trp, Tyr |
| X7 | Negative | Asp, Glu |
| X8 | Polar | Cys, Asp, Glu, His, Lys, |
| | | Asn, Gln, Arg, Ser, Thr |
| X9 | Positive | His, Lys, Arg |
| X10 | Small | Ala, Cys, Asp, Gly, Asn, |
| | | Pro, Ser, Thr, Val |
| X11 | Tiny | Ala, Gly, Ser |
| X12 | Turnlike | Ala, Cys, Asp, Glu, Gly, |
| | | His, Lys, Asn, Gln, Arg, |
| | | Ser, Thr |
| X13 | Asparagine-Aspartate | Asn, Asp |

*Abbreviations as adopted from http://smart.embl-heidelberg.de/SMART_DATA/alignments/consensus/grouping.html.

2. Overview

In certain aspects, the invention relates to methods and compositions employing human PEM-3-like nucleic acids and proteins. In certain aspects, the invention relates to novel associations between certain disease states and PEM-3-like nucleic acids and proteins. PEM-3-like polypeptides intersect with and regulate a wide range of key cellular functions that may be manipulated by affecting the level of and/or activity of PEM-3-like polypeptides. In certain aspects, the present invention provides methods for identifying diseases that are associated with defects in the PEM-3-like gene and methods for ameliorating such diseases. In further aspects, the invention provides nucleic acid agents (e.g., RNAi probes, antisense), antibody-related agents, small molecules and other agents that affect PEM-3-like protein function. In further aspects, the invention provides methods for identifying agents that affect PEM-3-like protein function. Other aspects and embodiments are described herein.

In certain aspects, the invention relates to PEM-3-like polypeptides that function as E3 enzymes in the ubiquitination system. Accordingly, downregulation or upregulation of PEM-3-like ubiquitin ligase activity can be used to manipulate biological processes that are affected by protein ubiquitination. Downregulation or upregulation may be achieved at any stage of PEM-3-like protein formation and regulation, including transcriptional, translational or post-translational regulation. For example, PEM-3-like transcript levels may be decreased by RNAi targeted at a PEM-3-like gene sequence. As another example, PEM-3-like ubiquitin ligase activity may be inhibited by contacting PEM-3-like protein with an antibody that binds to and interferes with a PEM-3 -like RING domain or a domain of PEM-3-like protein that mediates interaction with a target protein (a protein that is ubiquitinated at least in part because of PEM-3-like protein activity). As another example, PEM-3-like protein activity may be increased by causing increased expression of PEM-3-like polypeptides or an active portion thereof. A ubiquitin ligase, such as PEM-3-like protein, may participate in biological processes including, for example, one or more of the various stages of a viral lifecycle, such as viral entry into a cell, production of viral proteins, assembly of viral proteins and release of viral particles from the cell. PEM-3-like proteins may participate in diseases characterized by the accumulation of ubiquitinated proteins, such as dementias (e.g., Alzheimer's and Pick's), inclusion body myositis and myopathies, polyglucosan body myopathy, and certain forms of amyotrophic lateral sclerosis. PEM-3-like polypeptides may participate in diseases characterized by excessive or inappropriate ubiquitination and/or protein degradation. Certain PEM-3-like polypeptides function as ubiquitin ligases. Accordingly, aspects of the present invention permit one of ordinary skill in the art to identify diseases that are associated with an altered PEM-3-like ubiquitin ligase activity.

In certain embodiments, the application relates to PEM-3 -like polypeptides that are neddylated. In certain further embodiments, the application relates to PEM-3-like polypeptides that are involved in neddylation. NEDD8 is a member of ubiquitin-like proteins, which modify proteins in a manner similar to ubiquitination. Neddylation involves the activity of an E1, e.g., APP-BP1/Uba3, and an E2, e.g., UBC12. In certain embodiments, the application relates to a complex comprising PEM-3-like and NEDD8. In additional embodiments, the application relates to a complex comprising PEM-3-like and an E2, such as UBC12. In further embodiments, the application relates to fusion proteins comprising PEM-3-like and NEDD8 amino acid sequence. For example, the present application provides PEM-3-like polypeptide as a recombinant fusion protein which includes a second polypeptide portion, e.g., the second polypeptide portion is NEDD8.

In certain aspects, the invention relates to the discovery that certain PEM-3-like polypeptides are involved in viral maturation, including the production, post-translational processing, assembly and/or release of proteins in a viral particle. Accordingly, viral infections may be ameliorated by inhibiting an activity (e.g., ubiquitin ligase activity or target protein interaction) of PEM-3-like polypeptides, and in preferred embodiments, the virus is a virus that employs a Gag protein, such as HIV, SIV, Ebola or functional homologs such as VP40 for Ebola. Additional viral species are described in greater detail below.

The protein, SAM68, and homologous proteins containing a KH domain, play an important role in the post-transcriptional regulation of HIV-1 replication. These proteins are involved in the CRMI pathway and have been found to interact with viral RNA. CRM1 is a receptor protein normally involved in the nuclear export of certain RNAs and proteins. HIV-1 matrix (MA), the amino-terminal domain of the Pr55 gag polyprotein, is involved in directing unspliced viral RNA from the nucleus to the plasma membrane. Although MA does not contain the canonical leucine-rich nuclear export signal, nuclear export is mediated through the conserved CRM1p pathway (Dupont, S et al. (1999) Nature 402:681-685). Nuclear export of another retroviral Gag polyprotein, the Rous sarcoma virus Gag polyprotein, is mediated by a CRM1-dependent nuclear export pathway (Scheifele, L Z et al. (2002) Proc Natl Acad Sci USA 99:3944-3949). PEM-3-like protein bears a unique composition of KH domains and RING domains and is predicted to localize to the nucleoplasm and to the cytoplasm. While not wishing to be bound to mechanism, PEM-3-like polypeptides may be involved in the CRMl pathway and may play a role in the post-transcriptional regulation of HIV-1 and in the replication of other viruses.

3. Exemplary Nucleic Acids and Expression Vectors

In certain aspects the invention provides nucleic acids encoding PEM-3-like polypeptides, such as, for example, SEQ ID NOS: 23, 26 and 27. Nucleic acids of the invention are further understood to include nucleic acids that comprise variants of SEQ ID NOS: 22, 24 and 25. In certain aspects the invention provides methods employing nucleic acids encoding PEM-3-like polypeptides, such as, for example, SEQ ID NOS: 2, 4, 6, 8, 10, 12, 14, 16, 18, 20, 23, 26 and 27. Nucleic acids employed by methods of the invention are further understood to include nucleic acids that comprise variants of SEQ ID NOS: 1, 3, 5, 7, 9, 11, 13, 15, 17, 19, 22, 24 and 25. Variant nucleotide sequences include sequences that differ by one or more nucleotide substitutions, additions or deletions, such as allelic variants; and will, therefore, include coding sequences that differ from the nucleotide sequence of the coding sequence designated in SEQ ID NOS: 1, 3, 5, 7, 9, 11, 13, 15, 17, 19, 22, 24 and 25, e.g., due to the degeneracy of the genetic code. In other embodiments, variants will also include sequences that will hybridize under highly stringent conditions to a nucleotide sequence of a coding sequence designated in any of SEQ ID NOS: 1, 3, 5, 7, 9, 11, 13, 15, 17, 19, 22, 24 and 25. Preferred nucleic acids employed by methods of the invention are human PEM-3-like sequences, including, for example, any of SEQ ID NOS: 1, 3, 5, 7, 9, 11, 13, 15, 17, 19, 22, 24 and 25 and variants thereof and nucleic acids encoding an amino acid sequence selected from among SEQ ID NOS: 2, 4, 6, 8, 10, 12, 14, 16, 18, 20, 23, 26 and 27.

One of ordinary skill in the art will understand readily that appropriate stringency conditions which promote DNA hybridization can be varied. For example, one could perform the hybridization at 6.0× sodium chloride/sodium citrate (SSC) at about 45° C., followed by a wash of 2.0× SSC at 50° C. For example, the salt concentration in the wash step can be selected from a low stringency of about 2.0× SSC at 50° C. to a high stringency of about 0.2× SSC at 50° C. In addition, the temperature in the wash step can be increased from low stringency conditions at room temperature, about 22° C., to high stringency conditions at about 65° C. Both temperature and salt may be varied, or temperature or salt concentration may be held constant while the other variable is changed. In one embodiment, the invention provides nucleic acids which hybridize under low stringency conditions of 6×SSC at room temperature followed by a wash at 2×SSC at room temperature.

Isolated nucleic acids which differ from SEQ ID NOS: 22, 24 and 25 due to degeneracy in the genetic code are also within the scope of the invention. Likewise, isolated nucleic acids which differ from SEQ ID NOS: 1, 3, 5, 7, 9, 11, 13, 15, 17, 19, 22, 24 and 25 due to degeneracy in the genetic code are also within the scope being employed by methods of the invention. For example, a number of amino acids are designated by more than one triplet. Codons that specify the same amino acid, or synonyms (for example, CAU and CAC are synonyms for histidine) may result in “silent” mutations which do not affect the amino acid sequence of the protein. However, it is expected that DNA sequence polymorphisms that do lead to changes in the amino acid sequences of the subject proteins will exist among mammalian cells. One skilled in the art will appreciate that these variations in one or more nucleotides (up to about 3-5% of the nucleotides) of the nucleic acids encoding a particular protein may exist among individuals of a given species due to natural allelic variation. Any and all such nucleotide variations and resulting amino acid polymorphisms are within the scope of this invention. Optionally, a PEM-3-like nucleic acid used by a method of the invention will genetically complement a partial or complete PEM-3-like protein loss of function phenotype in a cell. For example, a PEM-3-like nucleic acid employed by a method of the invention may be expressed in a cell in which endogenous PEM-3-like protein has been reduced by RNAi, and the introduced PEM-3-like nucleic acid will mitigate a phenotype resulting from the RNAI. An exemplary PEM-3-like loss of function phenotype is a decrease in virus-like particle production in a cell transfected with a viral vector, optionally an HIV vector.

Another aspect of the invention relates to PEM-3-like nucleic acids that are used for antisense, RNAi or ribozymes. As used herein, nucleic acid therapy refers to administration or in situ generation of a nucleic acid or a derivative thereof which specifically hybridizes (e.g., binds) under cellular conditions with the cellular iRNA and/or genomic DNA encoding one of the subject PEM-3-like polypeptides so as to inhibit production of that protein, e.g., by inhibiting transcription and/or translation. The binding may be by conventional base pair complementarity, or, for example, in the case of binding to DNA duplexes, through specific interactions in the major groove of the double helix.

A nucleic acid therapy construct used by methods of the present invention can be delivered, for example, as an expression plasmid which, when transcribed in the cell, produces RNA which is complementary to at least a unique portion of the cellular mRNA which encodes a PEM-3-like polypeptide. Alternatively, the construct is an oligonucleotide which is generated ex vivo and which, when introduced into the cell causes inhibition of expression by hybridizing with the mRNA and/or genomic sequences encoding a PEM-3-like polypeptide. Such oligonucleotide probes are optionally modified oligonucleotide which are resistant to endogenous nucleases, e.g., exonucleases and/or endonucleases, and is therefore stable in vivo. Exemplary nucleic acid molecules for use as antisense oligonucleotides are phosphoramidate, phosphothioate and methylphosphonate analogs of DNA (see also U.S. Pat. Nos. 5,176,996; 5,264,564; and 5,256,775). Additionally, general approaches to constructing oligomers useful in nucleic acid therapy have been reviewed, for example, by van der Krol et al., (1988) Biotechniques 6:958-976; and Stein et al., (1988) Cancer Res 48:2659-2668.

Accordingly, methods of the invention make use of the modified oligomers that are useful in therapeutic, diagnostic, and research contexts. In therapeutic applications, the oligomers are utilized in a manner appropriate for nucleic acid therapy in general.

In addition to use in therapy, the oligomers employed by methods of the invention may be used as diagnostic reagents to detect the presence or absence of the PEM-3-like DNA or RNA sequences to which they specifically bind, such as for determining the level of expression of a gene of the invention or for determining whether a gene of the invention contains a genetic lesion.

In another aspect of the invention, the invention relates to methods employing nucleic acid that is provided in an expression vector comprising a nucleotide sequence encoding a subject PEM-3-like polypeptide and operably linked to at least one regulatory sequence. Regulatory sequences are art-recognized and are selected to direct expression of the PEM-3-like polypeptide. Accordingly, the term regulatory sequence includes promoters, enhancers and other expression control elements. Exemplary regulatory sequences are described in Goeddel; Gene Expression Technology: Methods in Enzymology, Academic Press, San Diego, Calif. (1990). For instance, any of a wide variety of expression control sequences that control the expression of a DNA sequence when operatively linked to it may be used in these vectors to express DNA sequences encoding a PEM-3-like polypeptide. Such useful expression control sequences, include, for example, the early and late promoters of SV40, tet promoter, adenovirus or cytomegalovirus immediate early promoter, the lac system, the trp system, the TAC or TRC system, T7 promoter whose expression is directed by T7 RNA polymerase, the major operator and promoter regions of phage lambda, the control regions for fd coat protein, the promoter for 3-phosphoglycerate ldnase or other glycolytic enzymes, the promoters of acid phosphatase, e.g., Pho5, the promoters of the yeast α-mating factors, the polyhedron promoter of the baculovirus system and other sequences known to control the expression of genes of prokaryotic or eukaryotic cells or their viruses, and various combinations thereof. It should be understood that the design of the expression vector may depend on such factors as the choice of the host cell to be transformed and/or the type of protein desired to be expressed. Moreover, the vector's copy number, the ability to control that copy number and the expression of any other protein encoded by the vector, such as antibiotic markers, should also be considered.

As will be apparent, the subject gene constructs can be used to cause expression of the subject PEM-3-like polypeptides in cells propagated in culture, e.g., to produce proteins or polypeptides, including fusion proteins or polypeptides, for purification.

This invention also pertains to the use of a host cell transfected with a recombinant gene including a coding sequence for one or more of the subject PEM-3-like polypeptides. The host cell may be any prokaryotic or eukaryotic cell. For example, a polypeptide of the present invention may be expressed in bacterial cells such as E. coli, insect cells (e.g., using a baculovirus expression system), yeast, or mammalian cells. Other suitable host cells are known to those skilled in the art.

Accordingly, the present invention further pertains to methods of producing the subject PEM-3-like polypeptides. For example, a host cell transfected with an expression vector encoding a PEM-3-like polypeptide can be cultured under appropriate conditions to allow expression of the polypeptide to occur. The polypeptide may be secreted and isolated from a mixture of cells and medium containing the polypeptide. Alternatively, the polypeptide may be retained cytoplasmically and the cells harvested, lysed and the protein isolated. A cell culture includes host cells, media and other byproducts. Suitable media for cell culture are well known in the art. The polypeptide can be isolated from cell culture medium, host cells, or both using techniques known in the art for purifying proteins, including ion-exchange chromatography, gel filtration chromatography, ultrafiltration, electrophoresis, and immunoaffinity purification with antibodies specific for particular epitopes of the polypeptide. In a preferred embodiment, the PEM-3-like polypeptide is a fusion protein containing a domain which facilitates its purification, such as a PEM-3-like-protein-GST fusion protein, PEM-3-like-protein-intein fusion protein, PEM-3-like-protein-cellulose binding domain fusion protein, PEM-3-like-protein-polyhistidine fusion protein, etc.

A nucleotide sequence encoding a PEM-3-like polypeptide can be used to produce a recombinant form of the protein via microbial or eukaryotic cellular processes. Ligating the polynucleotide sequence into a gene construct, such as an expression vector, and transforming or transfecting into hosts, either eukaryotic (yeast, avian, insect or mammalian) or prokaryotic (bacterial) cells, are standard procedures.

A recombinant PEM-3-like nucleic acid can be produced by ligating the cloned gene, or a portion thereof, into a vector suitable for expression in either prokaryotic cells, eukaryotic cells, or both. Expression vehicles for production of recombinant PEM-3-like polypeptides include plasmids and other vectors. For instance, suitable vectors for the expression of a PEM-3-like polypeptide include plasmids of the types: pBR322-derived plasmids, pEMBL-derived plasmids, pEX-derived plasmids, pBTac-derived plasmids and pUC-derived plasmids for expression in prokaryotic cells, such as E. coli.

A number of vectors exist for the expression of recombinant proteins in yeast. For instance, YEP24, YIP5, YEP51, YEP52, pYES2, and YRP17 are cloning and expression vehicles useful in the introduction of genetic constructs into S. cerevisiae (see, for example, Broach et al., (1983) in Experimental Manipulation of Gene Expression, ed. M. Inouye Academic Press, p. 83, incorporated by reference herein). These vectors can replicate in E. coli due the presence of the pBR322 ori, and in S. cerevisiae due to the replication determinant of the yeast 2 micron plasmid. In addition, drug resistance markers such as ampicillin can be used.

The preferred mammalian expression vectors contain both prokaryotic sequences to facilitate the propagation of the vector in bacteria, and one or more eukaryotic transcription units that are expressed in eukaryotic cells. The pcDNAI/amp, pcDNAI/neo, pRc/CMV, pSV2gpt, pSV2neo, pSV2-dhfr, pTk2, pRSVneo, pMSG, pSVT7, pko-neo and pHyg derived vectors are examples of mammalian expression vectors suitable for transfection of eukaryotic cells. Some of these vectors are modified with sequences from bacterial plasmids, such as pBR322, to facilitate replication and drug resistance selection in both prokaryotic and eukaryotic cells. Alternatively, derivatives of viruses such as the bovine papilloma virus (BPV-1), or Epstein-Barr virus (pHEBo, pREP-derived and p205) can be used for transient expression of proteins in eukaryotic cells. Examples of other viral (including retroviral) expression systems can be found below in the description of gene therapy delivery systems. The various methods employed in the preparation of the plasmids and transformation of host organisms are well known in the art. For other suitable expression systems for both prokaryotic and eukaryotic cells, as well as general recombinant procedures, see Molecular Cloning A Laboratory Manual, 2nd Ed., ed. by Sambrook, Fritsch and Maniatis (Cold Spring Harbor Laboratory Press, 1989) Chapters 16 and 17. In some instances, it may be desirable to express the recombinant PEM-3-like polypeptide by the use of a baculovirus expression system. Examples of such baculovirus expression systems include pVL-derived vectors (such as pVL1392, pVL1393 and pVL941), pAcUW-derived vectors (such as pAcUW1), and pBlueBac-derived vectors (such as the β-gal containing pBlueBac III.

It is well known in the art that a methionine at the N-terminal position can be enzymatically cleaved by the use of the enzyme methionine aminopeptidase (MAP). MAP has been cloned from E. coli (Ben-Bassat et al., (1987) J. Bacteriol. 169:751-757) and Salmonella typhimurium and its in vitro activity has been demonstrated on recombinant proteins (Miller et al., (1987) PNAS USA 84:2718-1722). Therefore, removal of an N-terminal methionine, if desired, can be achieved either in vivo by expressing such recombinant polypeptides in a host which produces MAP (e.g., E. coli or CM89 or S. cerevisiae), or in vitro by use of purified MAP (e.g., procedure of Miller et al.).

Alternatively, the coding sequences for the polypeptide can be incorporated as a part of a fusion gene including a nucleotide sequence encoding a different polypeptide. This type of expression system can be useful under conditions where it is desirable, e.g., to produce an immunogenic fragment of a PEM-3-like polypeptide. For example, the VP6 capsid protein of rotavirus can be used as an immunologic carrier protein for portions of polypeptide, either in the monomeric form or in the form of a viral particle. The nucleic acid sequences corresponding to the portion of the PEM-3-like polypeptide to which antibodies are to be raised can be incorporated into a fusion gene construct which includes coding sequences for a late vaccinia virus structural protein to produce a set of recombinant viruses expressing fusion proteins comprising a portion of the protein as part of the virion. The Hepatitis B surface antigen can also be utilized in this role as well. Similarly, chimeric constructs coding for fusion proteins containing a portion of a PEM-3-like polypeptide and the poliovirus capsid protein can be created to enhance immunogenicity (see, for example, EP Publication NO: 0259149; and Evans et al., (1989) Nature 339:385; Huang et al., (1988) J. Virol. 62:3855; and Schlienger et al., (1992) J. Virol. 66:2).

The Multiple Antigen Peptide system for peptide-based immunization can be utilized, wherein a desired portion of a PEM-3-like polypeptide is obtained directly from organo-chemical synthesis of the peptide onto an oligomeric branching lysine core (see, for example, Posnett et al., (1988) JBC 263:1719 and Nardelli et al., (1992) J. Immunol. 148:914). Antigenic determinants of a PEM-3-like polypeptide can also be expressed and presented by bacterial cells.

In another embodiment, a fusion gene coding for a purification leader sequence, such as a poly-(His)/enterolinase cleavage site sequence at the N-terminus of the desired portion of the recombinant protein, can allow purification of the expressed fusion protein by affinity chromatography using a Ni²⁺ metal resin. The purification leader sequence can then be subsequently removed by treatment with enterokinase to provide the purified PEM-3-like polypeptide (e.g., see Hochuli et al., (1987) J. Chromatography 411:177; and Janknecht et al., PNAS USA 88:8972).

Techniques for making fusion genes are well known. Essentially, the joining of various DNA fragments coding for different polypeptide sequences is performed in accordance with conventional techniques, employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed to generate a chimeric gene sequence (see, for example, Current Protocols in Molecular Biology, eds. Ausubel et al., John Wiley & Sons: 1992).

4. Exemplary Polypeptides

The present invention also makes available isolated and/or purified forms of PEM-3-like polypeptides, which are isolated from, or otherwise substantially free of, other intracellular proteins which might normally be associated with the protein or a particular complex including the protein. The present invention also makes available methods employing isolated and/or purified forms of PEM-3-like polypeptides, which are isolated from, or otherwise substantially free of, other intracellular proteins which might normally be associated with the protein or a particular complex including the protein. In certain embodiments, the PEM-3-like polypeptides have an amino acid sequence that is at least 60% identical to an amino acid sequence as set forth in any of SEQ ID NOS: 2, 4, 6, 8, 10, 12, 14, 16, 18, 20, 23, 26 and 27. In other embodiments, the polypeptide has an amino acid sequence at least 65%, 70%, 75%, 80%, 85%, 90%, 95%, 97%, 98%, 99% or 100% identical to an amino acid sequence as set forth in any of SEQ ID NOS: 2, 4, 6, 8, 10, 12, 14, 16, 18, 20, 23, 26 and 27.

Optionally, a method of the invention employing a PEM-3-like polypeptide will make use of the PEM-3-like polypeptide to function in place of an endogenous PEM-3-like polypeptide, for example by mitigating a partial or complete PEM-3-like loss of finction phenotype in a cell. For example, a PEM-3-like polypeptide may be produced in a cell in which endogenous PEM-3-like protein has been reduced by RNAi, and the introduced PEM-3-like polypeptide will mitigate a phenotype resulting from the RNAi. An exemplary PEM-3-like loss of function phenotype is a decrease in virus-like particle production in a cell transfected with a viral vector, optionally an HIV vector.

In certain embodiments, a PEM-3-like polypeptide of the invention interacts with a viral Gag protein. In additional embodiments, PEM-3-like polypeptides may also, or alternatively, function in ubiquitination in part through the activity of a RING domain.

In another aspect, the invention provides methods employing polypeptides that are agonists or antagonists of a PEM-3-like polypeptide. Variants and fragments of a PEM-3-like polypeptide may have a hyperactive or constitutive activity, or, alternatively, act to prevent PEM-3-like polypeptides from performing one or more functions. For example, a truncated form lacking one or more domain may have a dominant negative effect.

Another aspect of the invention relates to methods employing polypeptides derived from a full-length PEM-3-like polypeptide. Isolated peptidyl portions of the subject proteins can be obtained by screening polypeptides recombinantly produced from the corresponding fragment of the nucleic acid encoding such polypeptides. In addition, fragments can be chemically synthesized using techniques known in the art such as conventional Merrifield solid phase f-Moc or t-Boc chemistry. For example, any one of the subject proteins can be arbitrarily divided into fragments of desired length with no overlap of the fragments, or preferably divided into overlapping fragments of a desired length. The fragments can be produced (recombinantly or by chemical synthesis) and tested to identify those peptidyl fragments which can function as either agonists or antagonists of the formation of a specific protein complex, or more generally of a PEM-3-like protein complex, such as by microinjection assays.

It is also possible to modify the structure of PEM-3-like polypeptides for such purposes as enhancing therapeutic or prophylactic efficacy, or stability (e.g., ex vivo shelf life and resistance to proteolytic degradation in vivo). Such modified polypeptides, when designed to retain at least one activity of the naturally-occurring form of the protein, are considered functional equivalents of the PEM-3-like polypeptides described in more detail herein. Such modified polypeptides can be produced, for instance, by amino acid substitution, deletion, or addition.

For instance, it is reasonable to expect, for example, that an isolated replacement of a leucine with an isoleucine or valine, an aspartate with a glutamate, a threonine with a serine, or a similar replacement of an amino acid with a structurally related amino acid (i.e. conservative mutations) will not have a major effect on the biological activity of the resulting molecule. Conservative replacements are those that take place within a family of amino acids that are related in their side chains. Genetically encoded amino acids are can be divided into four families: (1) acidic=aspartate, glutamate; (2) basic=lysine, arginine, histidine; (3) nonpolar=alanine, valine, leucine, isoleucine, proline, phenylalanine, methionine, tryptophan; and (4) uncharged polar=glycine, asparagine, glutamine, cysteine, serine, threonine, tyrosine. Phenylalanine, tryptophan, and tyrosine are sometimes classified jointly as aromatic amino acids. In similar fashion, the amino acid repertoire can be, grouped as (1) acidic=aspartate, glutamate; (2) basic=lysine, arginine histidine, (3) aliphatic=glycine, alanine, valine, leucine, isoleucine, serine, threonine, with serine and threonine optionally be grouped separately as aliphatic-hydroxyl; (4) aromatic=phenylalanine, tyrosine, tryptophan; (5) amide=asparagine, glutamine; and (6) sulfur—containing=cysteine and methionine. (see, for example, Biochemistry, 2nd ed., Ed. by L. Stryer, W.H. Freeman and Co., 1981). Whether a change in the amino acid sequence of a polypeptide results in a functional homolog can be readily determined by assessing the ability of the variant polypeptide to produce a response in cells in a fashion similar to the wild-type protein. For instance, such variant forms of a PEM-3-like polypeptide can be assessed, e.g., for their ability to bind to another polypeptide, e.g., another PEM-3-like polypeptide or another protein involved in viral maturation. Polypeptides in which more than one replacement has taken place can readily be tested in the same manner.

This invention further contemplates a method of generating sets of combinatorial mutants of PEM-3-like polypeptides for use in aspects of the invention, as well as truncation mutants, and is especially useful for identifying potential variant sequences (e.g., homologs) that are functional in binding to a PEM-3-like polypeptide. The purpose of screening such combinatorial libraries is to generate, for example, PEM-3-like protein homologs which can act as either agonists or antagonist, or alternatively, which possess novel activities all together. Combinatorially-derived homologs can be generated which have a selective potency relative to a naturally occurring PEM-3-like polypeptide. Such proteins, when expressed from recombinant DNA constructs, can be used in gene therapy protocols.

Likewise, mutagenesis can give rise to homologs which have intracellular half-lives dramatically different than the corresponding wild-type protein. For example, the altered protein can be rendered either more stable or less stable to proteolytic degradation or other cellular process which result in destruction of, or otherwise inactivation of the PEM-3-like polypeptide of interest. Such homologs, and the genes which encode them, can be utilized to alter PEM-3-like protein levels by modulating the half-life of the protein. For instance, a short half-life can give rise to more transient biological effects and, when part of an inducible expression system, can allow tighter control of recombinant PEM-3-like protein levels within the cell. As above, such proteins, and particularly their recombinant nucleic acid constructs, can be used in gene therapy protocols.

In similar fashion, PEM-3-like protein homologs can be generated by the present combinatorial approach to act as antagonists, in that they are able to interfere with the ability of the corresponding wild-type protein to function.

In a representative embodiment of this method, the amino acid sequences for a population of PEM-3-like protein homologs are aligned, preferably to promote the highest homology possible. Such a population of variants can include, for example, homologs from one or more species, or homologs from the same species but which differ due to mutation. Amino acids which appear at each position of the aligned sequences are selected to create a degenerate set of combinatorial sequences. In a preferred embodiment, the combinatorial library is produced by way of a degenerate library of genes encoding a library of polypeptides which each include at least a portion of potential PEM-3-like sequences. For instance, a mixture of synthetic oligonucleotides can be enzymatically ligated into gene sequences such that the degenerate set of potential PEM-3-like nucleotide sequences are expressible as individual polypeptides, or alternatively, as a set of larger fusion proteins (e.g., for phage display).

There are many ways by which the library of potential homologs can be generated from a degenerate oligonucleotide sequence. Chemical synthesis of a degenerate gene sequence can be carried out in an automatic DNA synthesizer, and the synthetic genes then be ligated into an appropriate gene for expression. The purpose of a degenerate set of genes is to provide, in one mixture, all of the sequences encoding the desired set of potential PEM-3-like sequences. The synthesis of degenerate oligonucleotides is well known in the art (see for example, Narang, S A (1983) Tetrahedron 39:3; Itakura et al., (1981) Recombinant DNA, Proc. 3rd Cleveland Sympos. Macromolecules, ed. A G Walton, Amsterdam: Elsevier pp273-289; Itakura et al., (1984) Annu. Rev. Biochem. 53:323; Itakura et al., (1984) Science 198:1056; Ike et al., (1983) Nucleic Acid Res. 11:477). Such techniques have been employed in the directed evolution of other proteins (see, for example, Scott et al., (1990) Science 249:386-390; Roberts et al., (1992) PNAS USA 89:2429-2433; Devlin et al., (1990) Science 249: 404-406; Cwirla et al., (1990) PNAS USA 87: 6378-6382; as well as U.S. Pat. Nos: 5,223,409, 5,198,346, and 5,096,815).

Alternatively, other forms of mutagenesis can be utilized to generate a combinatorial library. For example, PEM-3-like homologs (both agonist and antagonist forms) can be generated and isolated from a library by screening using, for example, alanine scanning mutagenesis and the like (Ruf et al., (1994) Biochemistry 33:1565-1572; Wang et al., (1994) J. Biol. Chem. 269:3095-3099; Balint et al., (1993) Gene 137:109-118; Grodberg et al., (1993) Eur. J. Biochem. 218:597-601; Nagashima et al., (1993) J. Biol. Chem. 268:2888-2892; Lowman et al., (1991) Biochemistry 30:10832-10838; and Cunningham et al., (1989) Science 244:1081-1085), by linker scanning mutagenesis (Gustin et al., (1993) Virology 193:653-660; Brown et al., (1992) Mol. Cell Biol. 12:2644-2652; McKnight et al., (1982) Science 232:316); by saturation mutagenesis (Meyers et al., (1986) Science 232:613); by PCR mutagenesis (Leung et al., (1989) Method Cell Mol Biol 1:11-19); or by random mutagenesis, including chemical mutagenesis, etc. (Miller et al., (1992) A Short Course in Bacterial Genetics, CSHL Press, Cold Spring Harbor, N.Y.; and Greener et al., (1994) Strategies in Mol Biol 7:32-34). Linker scanning mutagenesis, particularly in a combinatorial setting, is an attractive method for identifying truncated (bioactive) forms of PEM-3-like polypeptides.

A wide range of techniques are known in the art for screening gene products of combinatorial libraries made by point mutations and truncations, and, for that matter, for screening cDNA libraries for gene products having a certain property. Such techniques will be generally adaptable for rapid screening of the gene libraries generated by the combinatorial mutagenesis of PEM-3-like homologs. The most widely used techniques for screening large gene libraries typically comprises cloning the gene library into replicable expression vectors, transforming appropriate cells with the resulting library of vectors, and expressing the combinatorial genes under conditions in which detection of a desired activity facilitates relatively easy isolation of the vector encoding the gene whose product was detected. Each of the illustrative assays described below are amenable to high through-put analysis as necessary to screen large numbers of degenerate sequences created by combinatorial mutagenesis techniques.

In an illustrative embodiment of a screening assay, candidate combinatorial gene products of one of the subject proteins are displayed on the surface of a cell or virus, and the ability of particular cells or viral particles to bind a PEM-3-like polypeptide is detected in a “panning assay”. For instance, a library of PEM-3-like variants can be cloned into the gene for a surface membrane protein of a bacterial cell (Ladner et al., WO 88/06630; Fuchs et al., (1991) Bio/Technology 9:1370-1371; and Goward et al., (1992) TIBS 18:136-140), and the resulting fusion protein detected by panning, e.g., using a fluorescently labeled molecule which binds the PEM-3-like polypeptide, to score for potentially functional homologs. Cells can be visually inspected and separated under a fluorescence microscope, or, where the morphology of the cell permits, separated by a fluorescence-activated cell sorter.

In similar fashion, the gene library can be expressed as a fusion protein on the surface of a viral particle. For instance, in the filamentous phage system, foreign peptide sequences can be expressed on the surface of infectious phage, thereby conferring two significant benefits. First, since these phage can be applied to affinity matrices at very high concentrations, a large number of phage can be screened at one time. Second, since each infectious phage displays the combinatorial gene product on its surface, if a particular phage is recovered from an affinity matrix in low yield, the phage can be amplified by another round of infection. The group of almost identical E. coli filamentous phages M13, fd, and fl are most often used in phage display libraries, as either of the phage gIII or gVIII coat proteins can be used to generate fusion proteins without disrupting the ultimate packaging of the viral particle (Ladner et al., PCT publication WO 90/02909; Garrard et al., PCT publication WO 92/09690; Marks et al., (1992) J. Biol. Chem. 267:16007-16010; Griffiths et al., (1993) EMBO J. 12:725-734; Clackson et al., (1991) Nature 352:624-628; and Barbas et al., (1992) PNAS USA 89:4457-4461).

The invention also provides for reduction of the subject PEM-3-like polypeptides employed in aspects of the invention to generate mimetics, e.g., peptide or non-peptide agents, which are able to mimic binding of the authentic protein to another cellular partner. Such mutagenic techniques as described above, as well as the thioredoxin system, are also particularly useful for mapping the determinants of a PEM-3-like polypeptide which participate in protein-protein interactions involved in, for example, binding of proteins involved in viral maturation to each other. To illustrate, the critical residues of a PEM-3-like polypeptide which are involved in molecular recognition of a substrate protein can be determined and used to generate PEM-3-like polypeptide-derived peptidomimetics which bind to the substrate protein, and by inhibiting PEM-3-like binding, act to inhibit its biological activity. By employing, for example, scanning mutagenesis to map the amino acid residues of a PEM-3-like polypeptide which are involved in binding to another polypeptide, peptidornimetic compounds can be generated which mimic those residues involved in binding. For instance, non-hydrolyzable peptide analogs of such residues can be generated using benzodiazepine (e.g., see Freidinger et al., in Peptides: Chemistry and Biology, G. R. Marshall ed., ESCOM Publisher: Leiden, Netherlands, 1988), azepine (e.g., see Huffman et al., in Peptides: Chemistry and Biology, G. R. Marshall ed., ESCOM Publisher: Leiden, Netherlands, 1988), substituted gamma lactam rings (Garvey et al., in Peptides: Chemistry and Biology, G. R. Marshall ed., ESCOM Publisher: Leiden, Netherlands, 1988), keto-methylene pseudopeptides (Ewenson et al., (1986) J. Med. Chem. 29:295; and Ewenson et al., in Peptides: Structure and Function (Proceedings of the 9th American Peptide Symposium) Pierce Chemical Co. Rockland, Ill., 1985), b-turn dipeptide cores (Nagai et al., (1985) Tetrahedron Lett 26:647; and Sato et al., (1986) J Chem Soc Perkin Trans 1:1231), and b-aminoalcohols (Gordon et al., (1985) Biochem Biophys Res Commun 126:419; and Dann et al., (1986) Biochem Biophys Res Commun 134:71).

5. Antibodies and Uses Thereof

Another aspect of the invention pertains to an antibody specifically reactive with a PEM-3-like polypeptide. For example, by using immunogens derived from a PEM-3-like polypeptide, e.g., based on the cDNA sequences, anti-protein/anti-peptide antisera or monoclonal antibodies can be made by standard protocols (See, for example, Antibodies: A Laboratory Manual ed. by Harlow and Lane (Cold Spring Harbor Press: 1988)). A mammal, such as a mouse, a hamster or rabbit can be immunized with an immunogenic form of the peptide (e.g., a PEM-3-like polypeptide or an antigenic fragment which is capable of eliciting an antibody response, or a fusion protein as described above). Techniques for conferring immunogenicity on a protein or peptide include conjugation to carriers or other techniques well known in the art. An immunogenic portion of a PEM-3-like polypeptide can be administered in the presence of adjuvant. The progress of immunization can be monitored by detection of antibody titers in plasma or serum. Standard ELISA or other immunoassays can be used with the immunogen as antigen to assess the levels of antibodies. In a preferred embodiment, the subject antibodies are immunospecific for antigenic determinants of a PEM-3-like polypeptide of a mammal, e.g., antigenic determinants of a protein set forth in any of SEQ ID NOS: 2, 4, 6, 8, 10, 12, 14, 16, 18, 20, 23, 26 or 27.

In one embodiment, antibodies are specific for a RING domain or a KH domain, and preferably the domain is part of a PEM-3-like polypeptide. In a more specific embodiment, the domain is part of an amino acid sequence set forth in any of SEQ ID NOS: 2, 4, 6, 8, 10, 12, 14, 16, 18, 20, 23, 26 or 27. In another embodiment, the antibodies are immunoreactive with one or more proteins having an amino acid sequence that is at least 80% identical to an amino acid sequence as set forth in SEQ ID NOS: 2, 4, 6, 8, 10, 12, 14, 16, 18,20,23,26 and/or 27. In other embodiments, an antibody is immunoreactive with one or more proteins having an amino acid sequence that is 85%, 90%, 95%, 98%, 99% or identical to an amino acid sequence as set forth in SEQ ID NOS: 2, 4, 6, 8, 10, 12, 14, 16, 18, 20, 23, 26 and/or 27.

Following immunization of an animal with an antigenic preparation of a PEM-3-like polypeptide, anti-PEM-3-like antisera can be obtained and, if desired, polyclonal anti-PEM-3-like antibodies isolated from the serum. To produce monoclonal antibodies, antibody-producing cells (lymphocytes) can be harvested from an immunized animal and fused by standard somatic cell fusion procedures with immortalizing cells such as myeloma cells to yield hybridoma cells. Such techniques are well known in the art, and include, for example, the hybridoma technique (originally developed by Kohler and Milstein, (1975) Nature, 256: 495-497), the human B cell hybridoma technique (Kozbar et al., (1983) Immunology Today, 4: 72), and the EBV-hybridoma technique to produce human monoclonal antibodies (Cole et al., (1985) Monoclonal Antibodies and Cancer Therapy, Alan R. Liss, Inc. pp. 77-96). Hybridoma cells can be screened immunochemically for production of antibodies specifically reactive with a mammalian PEM-3-like polypeptide of the present invention and monoclonal antibodies isolated from a culture comprising such hybridoma cells. In one embodiment anti-human PEM-3-like antibodies specifically react with the protein encoded by a nucleic acid having SEQ ID NOS: 1, 3, 5, 7, 9, 11, 13, 15, 17, 19, 22, 24 or 25.

The term antibody as used herein is intended to include fragments thereof which are also specifically reactive with one of the subject PEM-3-like polypeptides. Antibodies can be fragmented using conventional techniques and the fragments screened for utility in the same manner as described above for whole antibodies. For example, F(ab)₂ fragments can be generated by treating antibody with pepsin. The resulting F(ab)₂ fragment can be treated to reduce disulfide bridges to produce Fab fragments. The antibody of the present invention is further intended to include bispecific, single-chain, and chimeric and humanized molecules having affinity for a PEM-3-like polypeptide conferred by at least one CDR region of the antibody. In preferred embodiments, the antibodies, the antibody further comprises a label attached thereto and able to be detected (e.g., the label can be a radioisotope, fluorescent compound, enzyme or enzyme co-factor).

Anti-PEM-3-like antibodies can be used, e.g., to monitor PEM-3-like polypeptide levels in an individual, particularly the presence of PEM-3-like protein at the plasma membrane for determining whether or not said patient is infected with a virus such as an RNA virus, or allowing determination of the efficacy of a given treatment regimen for an individual afflicted with such a disorder. In addition, PEM-3-like polypeptides are expected to localize, occasionally, to the released viral particle. Viral particles may be collected and assayed for the presence of a PEM-3-like polypeptide. The level of PEM-3-like polypeptide may be measured in a variety of sample types such as, for example, cells and/or in bodily fluid, such as in blood samples.

Another application of anti-PEM-3-like antibodies of the present invention is in the immunological screening of cDNA libraries constructed in expression vectors such as gt11, gt18-23, ZAP, and ORF8. Messenger libraries of this type, having coding sequences inserted in the correct reading frame and orientation, can produce fusion proteins. For instance, gt11 will produce fusion proteins whose amino termini consist of β-galactosidase amino acid sequences and whose carboxy termini consist of a foreign polypeptide. Antigenic epitopes of a PEM-3-like polypeptide, e.g., other orthologs of a particular protein or other paralogs from the same species, can then be detected with antibodies, as, for example, reacting nitrocellulose filters lifted from infected plates with the appropriate anti-PEM-3-like antibodies. Positive phage detected by this assay can then be isolated from the infected plate. Thus, the presence of PEM-3-like homologs can be detected and cloned from other animals, as can alternate isoforms (including splice variants) from humans.

6. Homology Searching of Nucleotide and Polypeptide Sequences

The nucleotide or amino acid sequences of the invention may be used as query sequences against databases such as GenBank, SwissProt, BLOCKS, and Pima II. These databases contain previously identified and annotated sequences that can be searched for regions of homology (similarity) using BLAST, which stands for Basic Local Alignment Search Tool (Altschul S F (1993) J Mol Evol 36:290-300; Altschul, S F et al (1990) J Mol Biol 215:403-10).

BLAST produces alignments of both nucleotide and amino acid sequences to determine sequence similarity. Because of the local nature of the alignments, BLAST is especially useful in determining exact matches or in identifying homologs which may be of prokaryotic (bacterial) or eukaryotic (animal, fungal or plant) origin. Other algorithms such as the one described in Smith, R. F. and T. F. Smith (1992; Protein Engineering 5:35-51), incorporated herein by reference, can be used when dealing with primary sequence patterns and secondary structure gap penalties. As disclosed in this application, sequences have lengths of at least 49 nucleotides and no more than 12% uncalled bases (where N is recorded rather than A, C, G, or T).

The BLAST approach, as detailed in Karlin and Altschul (1993; Proc Nat Acad Sci 90:5873-7) and incorporated herein by reference, searches matches between a query sequence and a database sequence, to evaluate the statistical significance of any matches found, and to report only those matches which satisfy the user-selected threshold of significance. Preferably the threshold is set at 10-25 for nucleotides and 3-15 for peptides.

7. Transgenic Animals and Uses Thereof

Another aspect of the invention features transgenic non-human animals which express a heterologous PEM-3-like gene, preferentially a human PEM-3-like gene of the present invention, and/or which have had one or both copies of the endogenous PEM-3-like genes disrupted in at least one of the tissue or cell-types of the animal. Accordingly, the invention features an animal model for viral infection. In one embodiment, the transgenic non-human animals is a mammal such as a mouse, rat, rabbit, goat, sheep, dog, cat, cow, or non-human primate. Without being bound to theory, it is proposed that such an animal may be susceptible to infection with envelop viruses, retroid virus and RNA virus such as various rhabdoviruses, lentiviruses, and filoviruses. HIV Accordingly, such a transgenic animal may serve as a useful animal model to study the progression of diseases caused by such viruses. Alternatively, such an animal can be useful as a basis to introduce one or more other human transgenes, to create a transgenic animal carrying multiple human genes involved in infection caused by retroid viruses or other RNA viruses. Retroid viruses include lentiviruses such as HIV. Other RNA viruses include filoviruses such as Ebola virus. As a result of the introduction of multiple human transgenes, the transgenic animal may become susceptible to certain viral infection and therefore provide an useful animal model to study these viral infection.

In a preferred embodiment, the transgenic animal carrying human PEM-3-like gene is useful as a basis to introduce other human genes involved in HIV infection, such as Cyclin T1, CD34, CCR5, and fusin (CRCX4). In a further embodiment, the additional human transgene is a gene involved in a disease or condition that is associated with AIDS (e.g., hypertension, Kaposi's sarcoma, cachexia, etc.) Such an animal may be an useful animal model for studying HIV infection, AIDS and related disease development.

Another aspect of the present invention concerns transgenic animals which are comprised of cells (of that animal) which contain a transgene of the present invention and which preferably (though optionally) express an exogenous PEM-3-like protein in one or more cells in the animal. A PEM-3-like transgene can encode the wild-type form of the protein, or can encode homologs thereof, as well as antisense constructs. Moreover, it may be desirable to express the heterologous PEM-3-like transgene conditionally such that either the timing or the level of PEM-3-like gene expression can be regulated. Such conditional expression can be provided using prokaryotic promoter sequences which require prokaryotic proteins to be simultaneous expressed in order to facilitate expression of the PEM-3-like transgene. Exemplary promoters and the corresponding trans-activating prokaryotic proteins are given in U.S. Pat. No. 4,833,080.

Moreover, transgenic animals exhibiting tissue specific expression can be generated, for example, by inserting a tissue specific regulatory element, such as an enhancer, into the transgene. For example, the endogenous PEM-3-like gene promoter or a portion thereof can be replaced with another promoter and/or enhancer, e.g., a CMV or a Moloney murine leukemia virus (MLV) promoter and/or enhancer.

Alternatively, non-human transgenic animals that only express HIV transgenes in the brain can be generated using brain specific promoters (e.g., myelin basic protein (MBP) promoter, the neurofilament protein (NF-L) promoter, the gonadotropin-releasing hormone promoter, the vasopressin promoter and the neuron-specific enolase promoter, see So Forss-Petter et al., Neuron, 5, 187, (1990). Such animals can provide a useful in vivo model to evaluate the ability of a potential anti-HIV drug to cross the blood-brain barrier. Other target cells for which specific promoters can be used are, for example, macrophages, T cells and B cells. Other tissue specific promoters are well-known in the art, see e.g., R. Jaenisch, Science, 240, 1468 (1988).

Non-human transgenic animals containing an inducible PEM-3-like transgene can be generated using inducible regulatory elements (e.g., metallothionein promoter), which are well-known in the art. PEM-3-like transgene expression can then be initiated in these animals by administering to the animal a compound which induces gene expression (e.g., heavy metals). Another preferred inducible system comprises a tetracycline-inducible transcriptional activator (U.S. Pat. No. 5,654,168 issued Aug. 5, 1997 to Bujard and Gossen and U.S. Pat. No. 5,650,298 issued Jul. 22, 1997 to Bujard et al.).

In general, transgenic animal lines can be obtained by generating transgenic animals having incorporated into their genome at least one transgene, selecting at least one founder from these animals and breeding the founder or founders to establish at least one line of transgenic animals having the selected transgene incorporated into their genome.

Animals for obtaining eggs or other nucleated cells (e.g., embryonic stem cells) for generating transgenic animals can be obtained from standard commercial sources such as Charles River Laboratories (Wilmington, Mass.), Taconic (Germantown, N.Y.), Harlan Sprague Dawley (Indianapolis, Ind.).

Eggs can be obtained from suitable animals, e.g., by flushing from the oviduct or using techniques described in U.S. Pat. No. 5,489,742 issued Feb. 6, 1996 to Hammer and Taurog; U.S. Pat. No. 5,625,125 issued on Apr. 29, 1997 to Bennett et al.; Gordon et al., 1980, Proc. Natl. Acad. Sci. USA 77:7380-7384; Gordon & Puddle, 1981, Science 214: 1244-1246; U.S. Pat. No. 4,873,191 to T. E. Wagner and P. C. Hoppe; U.S. Pat. No. 5,604,131; Armstrong, et al. (1988) J. of Reproduction, 39:511 or PCT application No. PCT/FR93/00598 (WO 94/00568) by Mehtali et al. Preferably, the female is subjected to hormonal conditions effective to promote superovulation prior to obtaining the eggs.

Many techniques can be used to introduce DNA into an egg or other nucleated cell, including in vitro fertilization using sperm as a carrier of exogenous DNA (“sperm-mediated gene transfer”, e.g., Lavitrano et al., 1989, Cell 57: 717-723), microinjection, gene targeting (Thompson et al., 1989, Cell 56: 313-321), electroporation (Lo, 1983, Mol. Cell. Biol. 3: 1803-1814), transfection, or retrovirus mediated gene transfer (Van der Putten et al., 1985, Proc. Natl. Acad. Sci. USA 82: 6148-6152). For a review of such techniques, see Gordon (1989), Transgenic Animals, Intl. Rev. Cytol. 115:171-229.

Except for sperm-mediated gene transfer, eggs should be fertilized in conjunction with (before, during or after) other transgene transfer techniques. A preferred method for fertilizing eggs is by breeding the female with a fertile male. However, eggs can also be fertilized by in vitro fertilization techniques.

Fertilized, transgene containing eggs can than be transferred to pseudopregnant animals, also termed “foster mother animals”, using suitable techniques. Pseudopregnant animals can be obtained, for example, by placing 40-80 day old female animals, which are more than 8 weeks of age, in cages with infertile males, e.g., vasectomized males. The next morning females are checked for vaginal plugs. Females who have mated with vasectomized males are held aside until the time of transfer.

Recipient females can be synchronized, e.g., using GNRH agonist (GnRH-a): des-gly10, (D-Ala6)-LH-RH Ethylamide, SigmaChemical Co., St. Louis, Mo. Alternatively, a unilateral pregnancy can be achieved by a brief surgical procedure involving the “peeling” away of the bursa membrane on the left uterine horn. Injected embryos can then be transferred to the left uterine horn via the infundibulum. Potential transgenic founders can typically be identified immediately at birth from the endogenous litter mates. For generating transgenic animals from embryonic stem cells, see e.g., Teratocarcinomas and embryonic stem cells, a practical approach, ed. E. J. Robertson, (IRL Press 1987) or in Potter et al Proc. Natl. Acad. Sci. USA 81, 7161 (1984), the teachings of which are incorporated herein by reference.

Founders that express the gene can then bred to establish a transgenic line. Accordingly, founder animals can be bred, inbred, crossbred or outbred to produce colonies of animals of the present invention. Animals comprising multiple transgenes can be generated by crossing different founder animals (e.g., an HIV transgenic animal and a transgenic animal, which expresses human CD4), as well as by introducing multiple transgenes into an egg or embryonic cell as described above. Furthermore, embryos from A-transgenic animals can be stored as frozen embryos, which are thawed and implanted into pseudo-pregnant animals when needed (See e.g., Hirabayashi et al. (1997) Exp Anim 46: 111 and Anzai (1994) Jikken Dobutsu 43: 247).

The present invention provides for transgenic animals that carry the transgene in all their cells, as well as animals that carry the transgene in some, but not all cells, i.e., mosaic animals. The transgene can be integrated as a single transgene or in tandem, e.g., head to head tandems, or head to tail or tail to tail or as multiple copies.

The successful expression of the transgene can be detected by any of several means well known to those skilled in the art. Non-limiting examples include Northern blot, in situ hybridization of mRNA analysis, Western blot analysis, immunohistochemistry, and FACS analysis of protein expression.

In a further aspect, the invention features non-human animal cells containing a PEM-3-like transgene, preferentially a human PEM-3-like transgene. For example, the animal cell (e.g., somatic cell or germ cell (i.e. egg or sperm)) can be obtained from the transgenic animal. Transgenic somatic cells or cell lines can be used, for example, in drug screening assays. Transgenic germ cells, on the other hand, can be used in generating transgenic progeny, as described above.

The invention further provides methods for identifying (screening) or for determining the safety and/or efficacy of virus therapeutics, i.e. compounds which are useful for treating and/or preventing the development of diseases or conditions, which are caused by, or contributed to by viral infection (e.g., AIDS). In addition the assays are useful for further improving known anti-viral compounds, e.g, by modifying their structure to increase their stability and/or activity and/or toxicity.

In addition to providing cells for in vitro assays, the transgenic animals themselves can be used in in vivo assays to identify viral therapeutics. For example, the animals can be used in assays to identify compounds which reduce or inhibit any phase of the viral life cycle, e.g., expression of one or more viral genes, activity of one or more viral proteins, glycosylation of one or more viral proteins, processing of one or more viral proteins, viral replication, assembly of virions, and/or budding of infectious virions.

In an exemplary embodiment, the assay comprises administering a test compound to a transgenic animal of the invention infected with a virus including envelop viruses, DNA viruses, retrovirus and other RNA viruses, and comparing a phenotypic change in the animal relative to a transgenic animal which has not received the test compound. For example, where the animal is infected with HIV, the phenotypic change can be the amelioration in an AIDS related complex (ARC), cataracts, inflammatory lesions in the central nervous system (CNV), a mild kidney sclerotic lesion, or a skin lesion, such as psoratic dermatitis, hyperkerstotic lesions, Kaposi's sarcoma or cachexia. The effect of a compound on inhibition of Kaposi's sarcoma can be determined, as described, e.g., in PCT/US97/11202 (WO97/49373) by Gallo et al. These and other HIV related symptoms or phenotypes are further described in Leonard et al. (1988) Science 242:1665.

In another embodiment, the phenotypic change is release/budding of virus particles. In yet another embodiment, the phenotypic change is the number of CD4+ T cells or the ratio of CD4+ T cells versus CD8+ T cells. In HIV infected humans as well as in HIV transgenic mice, analysis of lymph nodes indicate that the number of CD4+ T cells decreases and the number of CD8+ T cells increases. Numbers of CD4+ and CD8+ T cells can be determined, for example, by indirect immunofluorescence and flow cytometry, as described, e.g., in Santoro et al., supra.

Alternatively, a phenotypic change, e.g., a change in the expression level of an HIV gene can be monitored. The HIV RNA can be selected from the group consisting of gag mRNA, gag-pro-pol mRNA, vif mRNA, vpr mRNA, tat mRNA, rev mRNA, vpu/env mRNA, nef mRNA, and vpx mRNA. The HIV protein can be selected from the group consisting of Pr55 Gag and fragments thereof (p17 MA, p24 CA, p7 NC, p1, p9, p6, and p2), Pr160 Gag-Pro-Pol, and fragments thereof (p10 PR, p5l RT, p66 RT, p32 IN), p23 Vif, p15 Vpr, p14 Tat, p19 Rev, p16 Vpu, gPr 160 Env or fragments thereof (gp120 SU and gp41TM), p27 Nef, and p14 Vpx. The level of any of these mRNAs or proteins can be determined in cells from a tissue sample, such as a skin biopsy, as described in, e.g., PCT/US97/11202 (W097/49373) by Gallo et al. Quantitation of HIV mRNA and protein is further described elsewhere herein and also in, e.g., Dickie et al. (1996) AIDS Res. Human Retroviruses 12:1103. In a preferred embodiment, the level of gp120 on the surface of PBMC is determined. This can be done, as described in the examples, e.g., by immunofluorescence on PBMC obtained from the animals.

A further phenotypic change is the production level or rate of viral particles in the serum and/or tissue of the animal. This can be determined, e.g., by determining reverse transcriptase (RT activity) or viral load as described elsewhere herein as well as in PCT/US97/11202 (WO97/49373) by Gallo et al., such as by determining p24 antigen.

Yet another phenotypic change, which can indicate HIV infection or AIDS progression is the production of inflammatory cytolines such as IL-6, IL-8 and TNF-.alpha.; thus, efficacy of a compound as an anti-HIV therapeutic can be assessed by ELISA tests for the reduction of serum levels of any or all of these cytokines.

A vaccine can be tested by administering a test antigen to a transgenic animal of the invention. The animal can optionally be boosted with the same or a different antigen. Such animal is then infected with a virus such as HIV. The production of viral particles or expression of viral proteins is then measured at various times following the administration of the test vaccine. A decrease in the amount of viral particles produced or viral expression will indicate that the test vaccine is efficient in reducing or inhibiting viral production and/or expression. The amount of antibody produced by the animal in response to the vaccine antigen can also be determined according to methods known in the art and provides a relative indication of the immunogenicity of the particular antigen.

Cells from the transgenic animals of the invention can be established in culture and immortalized to establish cell lines. For example, immortalized cell lines can be established from the livers of transgenic rats, as described in Bulera et al. (1997) Hepatology 25: 1192. Cell lines from other types of cells can be established according to methods known in the art.

In one cell-based assay, cells expressing a PEM-3-like transgene can be infected with a virus of interest and incubated in the presence a test compound or a control compound. The production of viral particles is then compared. This assay system thus provides a means of identifying molecular antagonists which, for example, function by interfering with viral release/budding.

Cell based assays can also be used to identify compounds which modulate expression of a viral gene, modulate translation of a viral mRNA, or which modulate the stability of a viral mRNA or protein. Accordingly, a cell which is capable of expressing a particular viral protein can be incubated with a test compound and the amount of the viral protein produced in the cell medium can be measured and compared to that produced from a cell which has not been contacted with the test compound. The specificity of the compound for regulating the expression of the particular virus gene can be confirmed by various control analyses, e.g., measuring the expression of one or more control genes. This type of cellular assay can be particularly useful for determining the efficacy of antisense molecules or ribozymes.

8. RNA Interference, Ribozymes Antisense and DNA Enzyme

In certain aspects, the invention relates to RNAi, ribozyme, antisense and other nucleic acid-related methods and compositions for manipulating (typically decreasing) a PEM-3-like protein activity. An exemplary RNAI target sequence is depicted in SEQ ID NO: 21.

Certain embodiments of the invention make use of materials and methods for effecting knockdown of one or more PEM-3-like genes by means of RNA interference (RNAi). RNAI is a process of sequence-specific post-transcriptional gene repression which can occur in eukaryotic cells. In general, this process involves degradation of an mRNA of a particular sequence induced by double-stranded RNA (dsRNA) that is homologous to that sequence. For example, the expression of a long dsRNA corresponding to the sequence of a particular single-stranded mRNA (ss mRNA) will labilize that message, thereby “interfering” with expression of the corresponding gene. Accordingly, any selected gene may be repressed by introducing a dsRNA which corresponds to all or a substantial part of the mRNA for that gene. It appears that when a long dsRNA is expressed, it is initially processed by a ribonuclease III into shorter dsRNA oligonucleotides of as few as 21 to 22 base pairs in length. Furthermore, Accordingly, RNAi may be effected by introduction or expression of relatively short homologous dsRNAs. Indeed the use of relatively short homologous dsRNAs may have certain advantages as discussed below.

Mammalian cells have at least two pathways that are affected by double-stranded RNA (dsRNA). In the RNAi (sequence-specific) pathway, the initiating dsRNA is first broken into short interfering (si) RNAs, as described above. The siRNAs have sense and antisense strands of about 21 nucleotides that form approximately 19 nucleotide si RNAs with overhangs of two nucleotides at each 3′ end. Short interfering RNAs are thought to provide the sequence information that allows a specific messenger RNA to be targeted for degradation. In contrast, the nonspecific pathway is triggered by dsRNA of any sequence, as long as it is at least about 30 base pairs in length. The nonspecific effects occur because dsRNA activates two enzymes: PKR, which in its active form phosphorylates the translation initiation factor eIF2 to shut down all protein synthesis, and 2′, 5′ oligoadenylate synthetase (2′, 5′-AS), which synthesizes a molecule that activates Rnase L, a nonspecific enzyme that targets all mRNAs. The nonspecific pathway may represents a host response to stress or viral infection, and, in general, the effects of the nonspecific pathway are preferably minimized under preferred methods of the present invention. Significantly, longer dsRNAs appear to be required to induce the nonspecific pathway and, accordingly, dsRNAs shorter than about 30 bases pairs are preferred to effect gene repression by RNAi (see Hunter et al. (1975) J Biol Chem 250: 409-17; Manche et al. (1992) Mol Cell Biol 12: 5239-48; Minks et al. (1979) J Biol Chem 254: 10180-3; and Elbashir et al. (2001) Nature 411: 494-8).

RNAi has been shown to be effective in reducing or eliminating the expression of a gene in a number of different organisms including Caenorhabditiis elegans (see e.g., Fire et al. (1998) Nature 391: 806-11), mouse eggs and embryos (Wianny et al. (2000) Nature Cell Biol 2: 70-5; Svoboda et al. (2000) Development 127: 4147-56), and cultured RAT-1 fibroblasts (Bahramina et al. (1999) Mol Cell Biol 19: 274-83), and appears to be an anciently evolved pathway available in eukaryotic plants and animals (Sharp (2001) Genes Dev. 15: 485-90). RNAI has proven to be an effective means of decreasing gene expression in a variety of cell types including HeLa cells, NIH/3T3 cells, COS cells, 293 cells and BHK-21 cells, and typically decreases expression of a gene to lower levels than that achieved using antisense techniques and, indeed, frequently eliminates expression entirely (see Bass (2001) Nature 411: 428-9). In mammalian cells, siRNAs are effective at concentrations that are several orders of magnitude below the concentrations typically used in antisense experiments (Elbashir et al. (2001) Nature 411: 494-8).

The double stranded oligonucleotides used to effect RNAi are preferably less than 30 base pairs in length and, more preferably, comprise about 25, 24, 23, 22, 21, 20, 19, 18 or 17 base pairs of ribonucleic acid. Optionally the dsRNA oligonucleotides of the invention may include 3′ overhang ends. Exemplary 2-nucleotide 3′ overhangs may be composed of ribonucleotide residues of any type and may even be composed of 2′-deoxythymidine resides, which lowers the cost of RNA synthesis and may enhance nuclease resistance of siRNAs in the cell culture medium and within transfected cells (see Elbashi et al. (2001) Nature 411: 494-8). Longer dsRNAs of 50, 75, 100 or even 500 base pairs or more may also be utilized in certain embodiments of the invention. Exemplary concentrations of dsRNAs for effecting RNAi are about 0.05 nM, 0.1 nM, 0.5 nM, 1.0 nM, 1.5 nM, 25 nM or 100 nM, although other concentrations may be utilized depending upon the nature of the cells treated, the gene target and other factors readily discernable the skilled artisan. Exemplary dsRNAs may be synthesized chemically or produced in vitro or in vivo using appropriate expression vectors. Exemplary synthetic RNAs include 21 nucleotide RNAs chemically synthesized using methods known in the art (e.g., Expedite RNA phophoramidites and thymidine phosphoramidite (Proligo, Germany). Synthetic oligonucleotides are preferably deprotected and gel-purified using methods known in the art (see e.g., Elbashir et al. (2001) Genes Dev. 15: 188-200). Longer RNAs may be transcribed from promoters, such as 17 RNA polymerase promoters, known in the art. A single RNA target, placed in both possible orientations downstream of an in vitro promoter, will transcribe both strands of the target to create a dsRNA oligonucleotide of the desired target sequence. Any of the above RNA species will be designed to include a portion of nucleic acid sequence represented in a PEM-3-like nucleic acid, such as, for example, a nucleic acid that hybridizes, under stringent and/or physiological conditions, to any of SEQ ID NOS: 1, 3, 5, 7, 9, 11, 13, 15, 17, 19, 22, 24 and 25 and complements thereof An exemplary RNAi target sequence is depicted in SEQ ID NO: 21. In certain embodiments, any of the above RNA species will be designed to include a portion of nucleic acid sequence represented in a PEM-3-like nucleic acid that encodes one or more N-terminal amino acids (e.g., one or more of the first 200 amino acids) of a PEM-3-like protein represented by any of SEQ ID NOS: 23, 26, and 27 or one or more of the nucleotides of the 5′ untranslated region of any of SEQ ID NOS: 22, 24, and 25.

The specific sequence utilized in design of the oligonucleotides may be any contiguous sequence of nucleotides contained within the expressed gene message of the target. Programs and algorithms, known in the art, may be used to select appropriate target sequences. In addition, optimal sequences may be selected utilizing programs designed to predict the secondary structure of a specified single stranded nucleic acid sequence and allowing selection of those sequences likely to occur in exposed single stranded regions of a folded mRNA. Methods and compositions for designing appropriate oligonucleotides may be found, for example, in U.S. Pat. Nos. 6,251,588, the contents of which are incorporated herein by reference. Messenger RNA (mRNA) is generally thought of as a linear molecule which contains the information for directing protein synthesis within the sequence of ribonucleotides, however studies have revealed a number of secondary and tertiary structures that exist in most mRNAs. Secondary structure elements in RNA are formed largely by Watson-Crick type interactions between different regions of the same RNA molecule. Important secondary structural elements include intramolecular double stranded regions, hairpin loops, bulges in duplex RNA and internal loops. Tertiary structural elements are formed when secondary structural elements come in contact with each other or with single stranded regions to produce a more complex three dimensional structure. A number of researchers have measured the binding energies of a large number of RNA duplex structures and have derived a set of rules which can be used to predict the secondary structure of RNA (see e.g., Jaeger et al. (1989) Proc. Natl. Acad. Sci. USA 86:7706 (1989); and Turner et al. (1988) Annu. Rev. Biophys. Biophys. Chem. 17:167) . The rules are useful in identification of RNA structural elements and, in particular, for identifying single stranded RNA regions which may represent preferred segments of the mRNA to target for silencing RNAi, ribozyme or antisense technologies. Accordingly, preferred segments of the mRNA target can be identified for design of the RNAi mediating dsRNA oligonucleotides as well as for design of appropriate ribozyme and hammerheadribozyme compositions of the invention.

The dsRNA oligonucleotides may be introduced into the cell by transfection with an heterologous target gene using carrier compositions such as liposomes, which are known in the art- e.g., Lipofectamine 2000 (Life Technologies) as described by the manufacturer for adherent cell lines. Transfection of dsRNA oligonucleotides for targeting endogenous genes may be carried out using Oligofectamine (Life Technologies). Transfection efficiency may be checked using fluorescence microscopy for mammalian cell lines after co-transfection of hGFP-encoding pAD3 (Kehlenback et al. (1998) J Cell Biol 141: 863-74). The effectiveness of the RNAi may be assessed by any of a number of assays following introduction of the dsRNAs. These include Western blot analysis using antibodies which recognize the PEM-3-like gene product following sufficient time for turnover of the endogenous pool after new protein synthesis is repressed, reverse transcriptase polymerase chain reaction and Northern blot analysis to determine the level of existing PEM-3-like target mRNA.

Further compositions, methods and applications of RNAi technology are provided in U.S. Application Pat. Nos. 6,278,039, 5,723,750 and 5,244,805, which are incorporated herein by reference.

Ribozyme molecules designed to catalytically cleave PEM-3-like mRNA transcripts can also be used to prevent translation of subject PEM-3-like mRNAs and/or expression of PEM-3-like protein (see, e.g., PCT International Publication WO90/11364, published Oct. 4, 1990; Sarver et al. (1990) Science 247:1222-1225 and U.S. Pat. No. 5,093,246). Ribozymes are enzymatic RNA molecules capable of catalyzing the specific cleavage of RNA. (For a review, see Rossi (1994) Current Biology 4: 469-471). The mechanism of ribozyme action involves sequence specific hybridization of the ribozyme molecule to complementary target RNA, followed by an endonucleolytic cleavage event. The composition of ribozyme molecules preferably includes one or more sequences complementary to a PEM-34-like mRNA, and the well known catalytic sequence responsible for mRNA cleavage or a functionally equivalent sequence (see, e.g., U.S. Pat. No. 5,093,246, which is incorporated herein by reference in its entirety).

While ribozymes that cleave mRNA at site specific recognition sequences can be used to destroy target mRNAs, the use of hammerhead ribozymes is preferred. Hammerhead ribozymes cleave mRNAs at locations dictated by flanking regions that form complementary base pairs with the target mRNA. Preferably, the target mRNA has the following sequence of two bases: 5′-UG-3′. The construction and production of hammerhead ribozymes is well known in the art and is described more fully in Haseloff and Gerlach ((1988) Nature 334:585-591; and see PCT Appln. No. WO89/05852, the contents of which are incorporated herein by reference). Hammerhead ribozyme sequences can be embedded in a stable RNA such as a transfer RNA (tRNA) to increase cleavage efficiency in vivo (Perriman et al. (1995) Proc. Natl. Acad. Sci. USA, 92: 6175-79; de Feyter, and Gaudron, Methods in Molecular Biology, Vol. 74, Chapter 43, “Expressing Ribozymes in Plants”, Edited by Turner, P. C, Humana Press Inc., Totowa, N.J.). In particular, RNA polymerase III-mediated expression of tRNA fusion ribozymes are well known in the art ( see Kawasaki et al. (1998) Nature 393: 284-9; Kuwabara et al. (1998) Nature Biotechnol. 16: 961-5; and Kuwabara et al. (1998) Mol. Cell 2: 617-27; Koseki et al. (1999) J Virol 73: 1868-77; Kuwabara et al. (1999) Proc Natl Acad Sci USA 96: 1886-91; Tanabe et al. (2000) Nature 406: 473-4). There are typically a number of potential hammerhead ribozyme cleavage sites within a given target cDNA sequence. Preferably the ribozyme is engineered so that the cleavage recognition site is located near the 5′ end of the target mRNA- to increase efficiency and minimize the intracellular accumulation of non-functional mRNA transcripts. Furthermore, the use of any cleavage recognition site located in the target sequence encoding different portions of the C-terminal amino acid domains of, for example, long and short forms of target would allow the selective targeting of one or the other form of the target, and thus, have a selective effect on one form of the target gene product.

Gene targeting ribozymes necessarily contain a hybridizing region complementary to two regions, each of at least 5 and preferably each 6, 7, 8, 9, 10, 11, 12, 13, 14, 15, 16, 17, 18, 19 or 20 contiguous nucleotides in length of a PEM-3-like mRNA, such as an mRNA of a sequence represented in any of SEQ ID NOS: 1, 3, 5, 7, 9, 11, 13, 15, 17, 19, 22, 24 and 25. In addition, ribozymes possess highly specific endoribonuclease activity, which autocatalytically cleaves the target sense mRNA. The present invention extends to ribozymes which hybridize to a sense mRNA encoding a PEM-3-like gene such as a therapeutic drug target candidate gene, thereby hybridising to the sense mRNA and cleaving it, such that it is no longer capable of being translated to synthesize a functional polypeptide product.

The ribozymes of the present invention also include RNA endoribonucleases (hereinafter “Cech-type ribozymes”) such as the one which occurs naturally in Tetrahymena thermophila (known as the IVS, or L-19 IVS RNA) and which has been extensively described by Thomas Cech and collaborators (Zaug, et al. (1984) Science 224:574-578; Zaug, et al. (1986) Science 231:470-475; Zaug, et al. (1986) Nature 324:429-433; published International patent application No. WO88/04300 by University Patents Inc.; Been, et al. (1986) Cell 47:207-216). The Cech-type ribozymes have an eight base pair active site which hybridizes to a target RNA sequence whereafter cleavage of the target RNA takes place. The invention encompasses those Cech-type ribozymes which target eight base-pair active site sequences that are present in a target gene or nucleic acid sequence.

Ribozymes can be composed of modified oligonucleotides (e.g., for improved stability, targeting, etc.) and should be delivered to cells which express the target gene in vivo. A preferred method of delivery involves using a DNA construct “encoding” the ribozyme under the control of a strong constitutive pol II or pol II promoter, so that transfected cells will produce sufficient quantities of the ribozyme to destroy endogenous target messages and inhibit translation. Because ribozymes, unlike antisense molecules, are catalytic, a lower intracellular concentration is required for efficiency.

In certain embodiments, a ribozyme may be designed by first identifying a sequence portion sufficient to cause effective knockdown by RNAi. The same sequence portion may then be incorporated into a ribozyme. In this aspect of the invention, the gene-targeting portions of the ribozyme or RNAi are substantially the same sequence of at least 5 and preferably 6, 7, 8, 9, 10, 11, 12, 13, 14, 15, 16, 17, 18, 19 or 20 or more contiguous nucleotides of a PEM-3-like nucleic acid, such as a nucleic acid of any of SEQ ID NOS: 1, 3, 5, 7, 9, 11, 13, 15, 17, 19, 22, 24 or 25. In a long target RNA chain, significant numbers of target sites are not accessible to the ribozyme because they are hidden within secondary or tertiary structures (Birikh et al. (1997) Eur J Biochem 245: 1-16). To overcome the problem of target RNA accessibility, computer generated predictions of secondary structure are typically used to identify targets that are most likely to be single-stranded or have an “open” configuration (see Jaeger et al. (1989) Methods Enzymol 183: 281-306). Other approaches utilize a systematic approach to predicting secondary structure which involves assessing a huge number of candidate hybridizing oligonucleotides molecules (see Milner et al. (1997) Nat Biotechnol 15: 537-41; and Patzel and Sczakiel (1998) Nat Biotechnol 16: 64-8). Additionally, U.S. Pat. No. 6,251,588, the contents of which are hereby incorporated herein, describes methods for evaluating oligonucleotide probe sequences so as to predict the potential for hybridization to a target nucleic acid sequence. The method of the invention provides for the use of such methods to select preferred segments of a target mRNA sequence that are predicted to be single-stranded and, further, for the opportunistic utilization of the same or substantially identical target mRNA sequence, preferably comprising about 10-20 consecutive nucleotides of the target mRNA, in the design of both the RNAi oligonucleotides and ribozymes of the invention.

A further aspect of the invention relates to the use of the isolated “antisense” nucleic acids to inhibit expression, e.g., by inhibiting transcription and/or translation of a subject PEM-3-like nucleic acid. The antisense nucleic acids may bind to the potential drug target by conventional base pair complementarity, or, for example, in the case of binding to DNA duplexes, through specific interactions in the major groove of the double helix. In general, these methods refer to the range of techniques generally employed in the art, and include any methods that rely on specific binding to oligonucleotide sequences.

An antisense construct of the present invention can be delivered, for example, as an expression plasmid which, when transcribed in the cell, produces RNA which is complementary to at least a unique portion of the cellular mRNA which encodes a PEM-3-like polypeptide. Alternatively, the antisense construct is an oligonucleotide probe, which is generated ex vivo and which, when introduced into the cell causes inhibition of expression by hybridizing with the mRNA and/or genomic sequences of a PEM-3-like nucleic acid. Such oligonucleotide probes are preferably modified oligonucleotides, which are resistant to endogenous nucleases, e.g., exonucleases and/or endonucleases, and are therefore stable in vivo. Exemplary nucleic acid molecules for use as antisense oligonucleotides are phosphoramidate, phosphothioate and methylphosphonate analogs of DNA (see also U.S. Pat. Nos. 5,176,996; 5,264,564; and 5,256,775). Additionally, general approaches to constructing oligomers useful in antisense therapy have been reviewed, for example, by Van der Krol et al. (1988) BioTechniques 6:958-976; and Stein et al. (1988) Cancer Res 48:2659-2668.

With respect to antisense DNA, oligodeoxyribonucleotides derived from the translation initiation site, e.g., between the −10 and +10 regions of the PEM-3-like gene, are preferred. Antisense approaches involve the design of oligonucleotides (either DNA or RNA) that are complementary to mRNA encoding the PEM-3-like polypeptide. The antisense oligonucleotides will bind to the mRNA transcripts and prevent translation. Absolute complementarity, although preferred, is not required. In the case of double-stranded antisense nucleic acids, a single strand of the duplex DNA may thus be tested, or triplex formation may be assayed. The ability to hybridize will depend on both the degree of complementarity and the length of the antisense nucleic acid. Generally, the longer the hybridizing nucleic acid, the more base mismatches with an RNA it may contain and still form a stable duplex (or triplex, as the case may be). One skilled in the art can ascertain a tolerable degree of mismatch by use of standard procedures to determine the melting point of the hybridized complex.

Oligonucleotides that are complementary to the 5′ end of the mRNA, e.g., the 5′ untranslated sequence up to and including the AUG initiation codon, should work most efficiently at inhibiting translation. However, sequences complementary to the 3′ untranslated sequences of mRNAs have recently been shown to be effective at inhibiting translation of mRNAs as well. (Wagner, R. 1994. Nature 372:333). Therefore, oligonucleotides complementary to either the 5′ or 3′ untranslated, non-coding regions of a gene could be used in an antisense approach to inhibit translation of that mRNA. Oligonucleotides complementary to the 5′ untranslated region of the mRNA should include the complement of the AUG start codon. Antisense oligonucleotides complementary to mRNA coding regions are less efficient inhibitors of translation but could also be used in accordance with the invention. Whether designed to hybridize to the 5′, 3′ or coding region of mRNA, antisense nucleic acids should be at least six nucleotides in length, and are preferably less that about 100 and more preferably less than about 50, 25, 17 or 10 nucleotides in length.

It is preferred that in vitro studies are first performed to quantitate the ability of the antisense oligonucleotide to inhibit gene expression. It is preferred that these studies utilize controls that distinguish between antisense gene inhibition and nonspecific biological effects of oligonucleotides. It is also preferred that these studies compare levels of the target RNA or protein with that of an internal control RNA or protein. Results obtained using the antisense oligonucleotide may be compared with those obtained using a control oligonucleotide. It is preferred that the control oligonucleotide is of approximately the same length as the test oligonucleotide and that the nucleotide sequence of the oligonucleotide differs from the antisense sequence no more than is necessary to prevent specific hybridization to the target sequence.

The antisense oligonucleotides can be DNA or RNA or chimeric mixtures or derivatives or modified versions thereof, single-stranded or double-stranded. The oligonucleotide can be modified at the base moiety, sugar moiety, or phosphate backbone, for example, to improve stability of the molecule, hybridization, etc. The oligonucleotide may include other appended groups such as peptides (e.g., for targeting host cell receptors), or agents facilitating transport across the cell membrane (see, e.g., Letsinger et al., 1989, Proc. Natl. Acad. Sci. U.S.A. 86:6553-6556; Lemaitre et al., 1987, Proc. Natl. Acad. Sci. 84:648-652; PCT Publication No. W088/09810, published December 15, 1988) or the blood- brain barrier (see, e.g., PCT Publication No. W089/10134, published Apr. 25, 1988), hybridization-triggered cleavage agents. (See, e.g., Krol et al., 1988, BioTechniques 6:958-976) or intercalating agents. (See, e.g., Zon, 1988, Pharm. Res. 5:539-549). To this end, the oligonucleotide may be conjugated to another molecule, e.g., a peptide, hybridization triggered cross-linking agent, transport agent, hybridization-triggered cleavage agent, etc.

The antisense oligonucleotide may comprise at least one modified base moiety which is selected from the group including but not limited to 5-fluorouracil, 5-bromouracil, 5-chlorouracil, 5-iodouracil, hypoxanthine, xantine, 4-acetylcytosine, 5-(carboxyhydroxytiethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine.

The antisense oligonucleotide may also comprise at least one modified sugar moiety selected from the group including but not limited to arabinose, 2-fluoroarabinose, xylulose, and hexose.

The antisense oligonucleotide can also contain a neutral peptide-like backbone. Such molecules are termed peptide nucleic acid (PNA)-oligomers and are described, e.g., in Perry-O'Keefe et al. (1996) Proc. Natl. Acad. Sci. U.S.A. 93:14670 and in Eglom et al. (1993) Nature 365:566. One advantage of PNA oligomers is their capability to bind to complementary DNA essentially independently from the ionic strength of the medium due to the neutral backbone of the DNA. In yet another embodiment, the antisense oligonucleotide comprises at least one modified phosphate backbone selected from the group consisting of a phosphorothioate, a phosphorodithioate, a phosphoramidothioate, a phosphoramidate, a phosphordiamidate, a methylphosphonate, an alkyl phosphotriester, and a formacetal or analog thereof.

In yet a further embodiment, the antisense oligonucleotide is an alpha-anomeric oligonucleotide. An alpha-anomeric oligonucleotide forms specific double-stranded hybrids with complementary RNA in which, contrary to the usual antiparallel orientation, the strands run parallel to each other (Gautier et al., 1987, Nucl. Acids Res. 15:6625-6641). The oligonucleotide is a 2′-0-methylribonucleotide (Inoue et al., 1987, Nucl. Acids Res. 15:6131-6148), or a chimeric RNA-DNA analogue (Inoue et al., 1987, FEBS Lett. 215:327-330).

While antisense nucleotides complementary to the coding region of a PEM-3-like mRNA sequence can be used, those complementary to the transcribed untranslated region may also be used.

In certain instances, it may be difficult to achieve intracellular concentrations of the antisense sufficient to suppress translation of endogenous mRNAs. Therefore a preferred approach utilizes a recombinant DNA construct in which the antisense oligonucleotide is placed under the control of a strong pol III or pol II promoter. The use of such a construct to transfect target cells will result in the transcription of sufficient amounts of single stranded RNAs that will form complementary base pairs with the endogenous potential drug target transcripts and thereby prevent translation. For example, a vector can be introduced such that it is taken up by a cell and directs the transcription of an antisense RNA. Such a vector can remain episomal or become chromosomally integrated, as long as it can be transcribed to produce the desired antisense RNA. Such vectors can be constructed by recombinant DNA technology methods standard in the art. Vectors can be plasmid, viral, or others known in the art, used for replication and expression in mammalian cells. Expression of the sequence encoding the antisense RNA can be by any promoter known in the art to act in mammalian, preferably human cells. Such promoters can be inducible or constitutive. Such promoters include but are not limited to: the SV40 early promoter region (Bernoist and Chambon, 1981, Nature 290:304-310), the promoter contained in the 3′ long terminal repeat of Rous sarcoma virus (Yamarnoto et al., 1980, Cell 22:787-797), the herpes thymidine kinase promoter (Wagner et al., 1981, Proc. Natl. Acad. Sci. U.S.A. 78:1441-1445), the regulatory sequences of the metallothionein gene (Brinster et al, 1982, Nature 296:39-42), etc. Any type of plasmid, cosmid, YAC or viral vector can be used to prepare the recombinant DNA construct, which can be introduced directly into the tissue site.

Alternatively, PEM-3-like gene expression can be reduced by targeting deoxyribonucleotide sequences complementary to the regulatory region of the gene (i.e., the promoter and/or enhancers) to form triple helical structures that prevent transcription of the gene in target cells in the body. (See generally, Helene, C. 1991, Anticancer Drug Des., 6(6):569-84; Helene, C., et al., 1992, Ann. N.Y. Acad. Sci., 660:27-36; and Maher, L. J., 1992, Bioassays 14(12):807-15).

Nucleic acid molecules to be used in triple helix formation for the inhibition of transcription are preferably single stranded and composed of deoxyribonucleotides. The base composition of these oligonucleotides should promote triple helix formation via Hoogsteen base pairing rules, which generally require sizable stretches of either purines or pyrimidines to be present on one strand of a duplex. Nucleotide sequences may be pyrimidine-based, which will result in TAT and CGC triplets across the three associated strands of the resulting triple helix. The pyrimidine-rich molecules provide base complementarity to a purine-rich region of a single strand of the duplex in a parallel orientation to that strand. In addition, nucleic acid molecules may be chosen that are purine- rich, for example, containing a stretch of G residues. These molecules will form a triple helix with a DNA duplex that is rich in GC pairs, in which the majority of the purine residues are located on a single strand of the targeted duplex, resulting in CGC triplets across the three strands in the triplex.

Alternatively, the potential PEM-3-like sequences that can be targeted for triple helix formation may be increased by creating a so called “switchback” nucleic acid molecule. Switchback molecules are synthesized in an alternating 5′-3′, 3′-5′ manner, such that they base pair with first one strand of a duplex and then the other, eliminating the necessity for a sizable stretch of either purines or pyrimidines to be present on one strand of a duplex.

A further aspect of the invention relates to the use of DNA enzymes to inhibit expression of a PEM-3-like gene. DNA enzymes incorporate some of the mechanistic features of both antisense and ribozyme technologies. DNA enzymes are designed so that they recognize a particular target nucleic acid sequence, much like an antisense oligonucleotide, however much like a ribozyme they are catalytic and specifically cleave the target nucleic acid.

There are currently two basic types of DNA enzymes, and both of these were identified by Santoro and Joyce (see, for example, U.S. Pat. No. 6,110,462). The 10-23 DNA enzyme comprises a loop structure which connect two arms. The two arms provide specificity by recognizing the particular target nucleic acid sequence while the loop structure provides catalytic function under physiological conditions.

Briefly, to design an ideal DNA enzyme that specifically recognizes and cleaves a target nucleic acid, one of skill in the art must first identify the unique target sequence. This can be done using the same approach as outlined for antisense oligonucleotides. Preferably, the unique or substantially sequence is a G/C rich of approximately 18 to 22 nucleotides. High G/C content helps insure a stronger interaction between the DNA enzyme and the target sequence.

When synthesizing the DNA enzyme, the specific antisense recognition sequence that will target the enzyme to the message is divided so that it comprises the two arms of the DNA enzyme, and the DNA enzyme loop is placed between the two specific arms.

Methods of making and administering DNA enzymes can be found, for example, in U.S. Pat. No. 6,110,462. Similarly, methods of delivery DNA ribozymes in vitro or in vivo include methods of delivery RNA ribozyme, as outlined in detail above. Additionally, one of skill in the art will recognize that, like antisense oligonucleotide, DNA enzymes can be optionally modified to improve stability and improve resistance to degradation.

Antisense RNA and DNA, ribozyme, RNAi and triple helix molecules of the invention may be prepared by any method known in the art for the synthesis of DNA and RNA molecules. These include techniques for chemically synthesizing oligodeoxyribonucleotides and oligoribonucleotides well known in the art such as for example solid phase phosphoramidite chemical synthesis. Alternatively, RNA molecules may be generated by in vitro and in vivo transcription of DNA sequences encoding the antisense RNA molecule. Such DNA sequences may be incorporated into a wide variety of vectors which incorporate suitable RNA polymerase promoters such as the T7 or SP6 polymerase promoters. Alternatively, antisense cDNA constructs that synthesize antisense RNA constitutively or inducibly, depending on the promoter used, can be introduced stably into cell lines. Moreover, various well-known modifications to nucleic acid molecules may be introduced as a means of increasing intracellular stability and half-life. Possible modifications include but are not limited to the addition of flanking sequences of ribonucleotides or deoxyribonucleotides to the 5′ and/or 3′ ends of the molecule or the use of phosphorothioate or 2′ O-methyl rather than phosphodiesterase linkages within the oligodeoxyribonucleotide backbone.

9. Drug Screening Assays

In certain aspects, the present invention also provides assays for identifying therapeutic agents which either interfere with or promote PEM-3-like protein function. In certain embodiments, agents of the invention are antiviral agents, optionally interfering with viral maturation, and preferably where the virus is a retrovirus, rhabdovirus or filovirus. In certain preferred embodiments, an antiviral agent interferes with the ubiquitin ligase catalytic activity of a PEM-3-like protein (e.g., PEM-3-like auto-ubiquitination or transfer to a target protein). In certain preferred embodiments, an antiviral agent interferes with the interaction between PEM-3-like protein and a target polypeptide. In certain embodiments, agents of the invention modulate the ubiquitin ligase activity of a PEM-3-like polypeptide and may be used to treat certain diseases related to ubiquitin ligase activity.

In certain embodiments, the invention provides assays to identify, optimize or otherwise assess agents that increase or decrease a ubiquitin-related activity of a PEM-3-like polypeptide. Ubiquitin-related activities of PEM-3-like polypeptides may include the self-ubiquitination activity of a PEM-3-like polypeptide, generally involving the transfer of ubiquitin from an E2 enzyme to the PEM-3-like polypeptide, and the ubiquitination of a target protein, generally involving the transfer of a ubiquitin from a PEM-3-like polypeptide to the target protein. In certain embodiments, a PEM-3-like protein activity is mediated, at least in part, by a PEM-3-like RING domain.

In certain embodiments, an assay comprises forming a mixture comprising a PEM-3-like polypeptide, an E2 polypeptide and a source of ubiquitin (which may be the E2 polypeptide pre-complexed with ubiquitin). Optionally the mixture comprises an E1 polypeptide and optionally the mixture comprises a target polypeptide. Additional components of the mixture may be selected to provide conditions consistent with the ubiquitination of the PEM-3-like polypeptide. One or more of a variety of parameters may be detected, such as PEM-3-like-ubiquitin conjugates, E2-ubiquitin thioesters, free ubiquitin and target polypeptide-ubiquitin complexes. The term “detect” is used herein to include a determination of the presence or absence of the subject of detection (e.g., PEM-3-like-ubiqutin, E2-ubiquitin, etc.), a quantitative measure of the amount of the subject of detection, or a mathematical calculation, based on the detection of other parameters, of the presence, absence or amount of the subject of detection. The term “detect” includes the situation wherein the subject of detection is determined to be absent or below the level of sensitivity. Detection may comprise detection of a label (e.g., fluorescent label, radioisotope label, and other described below), resolution and identification by size (e.g., SDS-PAGE, mass spectroscopy), purification and detection, and other methods that, in view of this specification, will be available to one of skill in the art. For instance, radioisotope labeling may be measured by scintillation counting, or by densitometry after exposure to a photographic emulsion, or by using a device such as a Phosphorimager. Likewise, densitometry may be used to measure bound ubiquitin following a reaction with an enzyme label substrate that produces an opaque product when an enzyme label is used. In a preferred embodiment, an assay comprises detecting the PEM-3-like-ubiquitin complex. In a screening assay, a test agent is added to the mixture. The parameter(s) detected in a screening assay may be compared to a suitable reference. A suitable reference may be an assay run previously, in parallel or later that omits the test agent. A suitable reference may also be an average of previous measurements in the absence of the test agent.

In certain embodiments, an assay comprises forming a mixture comprising a PEM-3-like polypeptide, a target polypeptide and a source of ubiquitin (which may be the PEM-3-like polypeptide pre-complexed with ubiquitin). Optionally the mixture comprises an E1 and/or E2 polypeptide and optionally the mixture comprises an E2-ubiquitin complex. Additional components of the mixture may be selected to provide conditions consistent with the ubiquitination of the target polypeptide. One or more of a variety of parameters may be detected, such as PEM-3-like-ubiquitin complexes and target polypeptide-ubiquitin complexes. In a preferred embodiment, an assay comprises detecting the target polypeptide-ubiquitin complex. In a screening assay, a test agent is added to the mixture. The parameter(s) detected in a screening assay may be compared to a suitable reference, as described above. In certain preferred embodiments, a screening assay for an antiviral agent employs a target polypeptide comprising an L domain, and preferably an HIV L domain.

In certain embodiments, an assay is performed in a high-throughput format. For example, one of the components of a mixture may be affixed to a solid substrate and one or more of the other components is labeled. For example, the PEM-3-like polypeptide may be affixed to a surface, such as a 96-well plate, and the ubiquitin is in solution and labeled. An E2 and E1 are also in solution, and the PEM-3-like-ubiquitin complex formation may be measured by washing the solid surface to remove uncomplexed labeled ubiquitin and detecting the ubiquitin that remains bound. Other variations may be used. For example, the amount of ubiquitin in solution may be detected. In certain embodiments, the formation of ubiquitin complexes may be measured by an interactive technique, such as FRET, wherein a ubiquitin is labeled with a first label and the desired complex partner (e.g., PEM-3-like polypeptide or target polypeptide) is labeled with a second label, wherein the first and second label interact when they come into close proximity to produce an altered signal. In FRET, the first and second labels are fluorophores. FRET is described in greater detail below. The formation of polyubiquitin complexes may be performed by mixing two or more pools of differentially labeled ubiquitin that interact upon formation of a polyubiquitin (see, e.g., US Patent Publication 20020042083). High-throughput may be achieved by performing an interactive assay, such as FRET, in solution as well. In addition, if a polypeptide in the mixture, such as the PEM-3-like polypeptide or target polypeptide, is readily purifiable (e.g., with a specific antibody or via a tag such as biotin, FLAG, polyhistidine, etc.), the reaction may be performed in solution and the tagged polypeptide rapidly isolated, along with any polypeptides, such as ubiquitin, that are associated with the tagged polypeptide. Proteins may also be resolved by SDS-PAGE for detection.

In certain embodiments, the ubiquitin is labeled, either directly or indirectly. This typically allows for easy and rapid detection and measurement of ligated ubiquitin, making the assay useful for high-throughput screening applications. As described above, certain embodiments may employ one or more tagged or labeled proteins. A “tag” is meant to include moieties that facilitate rapid isolation of the tagged polypeptide. A tag may be used to facilitate attachment of a polypeptide to a surface. A “label” is meant to include moieties that facilitate rapid detection of the labeled polypeptide. Certain moieties may be used both as a label and a tag (e.g., epitope tags that are readily purified and detected with a well-characterized antibody). Biotinylation of polypeptides is well known, for example, a large number of biotinylation agents are known, including amine-reactive and thiol-reactive agents, for the biotinylation of proteins, nucleic acids, carbohydrates, carboxylic acids; see chapter 4, Molecular Probes Catalog, Haugland, 6th Ed. 1996, hereby incorporated by reference. A biotinylated substrate can be attached to a biotinylated component via avidin or streptavidin. Similarly, a large number of haptenylation reagents are also known.

An “E1” is a ubiquitin activating enzyme. In a preferred embodiment, E1 is capable of transferring ubiquitin to an E2. In a preferred embodiment, E1 forms a high energy thiolester bond with ubiquitin, thereby “activating” the ubiquitin. An “E2” is a ubiquitin carrier enzyme (also known as a ubiquitin conjugating enzyme). In a preferred embodiment, ubiquitin is transferred from E1 to E2. In a preferred embodiment, the transfer results in a thiolester bond formed between E2 and ubiquitin. In a preferred embodiment, E2 is capable of transferring ubiquitin to a PEM-3-like polypeptide.

In an alternative embodiment, a PEM-3-like polypeptide, E2 or target polypeptide is bound to a bead, optionally with the assistance of a tag. Following ligation, the beads may be separated from the unbound ubiquitin and the bound ubiquitin measured. In a preferred embodiment, PEM-3-like polypeptide is bound to beads and the composition used includes labeled ubiquitin. In this embodiment, the beads with bound ubiquitin may be separated using a fluorescence-activated cell sorting (FACS) machine. Methods for such use are described in U.S. patent application Ser. No. 09/047,119, which is hereby incorporated in its entirety. The amount of bound ubiquitin can then be measured.

In a screening assay, the effect of a test agent may be assessed by, for example, assessing the effect of the test agent on kinetics, steady-state and/or endpoint of the reaction.

The components of the various assay mixtures provided herein may be combined in varying amounts. In a preferred embodiment, ubiquitin (or E2 complexed ubiquitin) is combined at a final concentration of from 5 to 200 ng per 100 microliter reaction solution. Optionally El is used at a final concentration of from 1 to 50 ng per 100 microliter reaction solution. Optionally E2 is combined at a final concentration of 10 to 100 ng per 100 .mu.l reaction solution, more preferably 10-50 ng per 100 microliter reaction solution. In a preferred embodiment, PEM-3-like polypeptide is combined at a final concentration of from 1 ng to 500 ng per 100 microliter reaction solution.

Generally, an assay mixture is prepared so as to favor ubiquitin ligase activity and/or ubiquitination acitivty. Generally, this will be physiological conditions, such as 50-200 mM salt (e.g., NaCl, KCl), pH of between 5 and 9, and preferably between 6 and 8. Such conditions may be optimized through trial and error. Incubations may be performed at any temperature which facilitates optimal activity, typically between 4 and 40 degrees C. Incubation periods are selected for optimum activity, but may also be optimized to facilitate rapid high through put screening. Typically between 0.5 and 1.5 hours will be sufficient. A variety of other reagents may be included in the compositions. These include reagents like salts, solvents, buffers, neutral proteins, e.g., albumin, detergents, etc. which may be used to facilitate optimal ubiquitination enzyme activity and/or reduce non-specific or background interactions. Also reagents that otherwise improve the efficiency of the assay, such as protease inhibitors, nuclease inhibitors, anti-microbial agents, etc., may be used. The compositions will also preferably include adenosine tri-phosphate (ATP). The mixture of components may be added in any order that promotes ubiquitin ligase activity or optimizes identification of candidate modulator effects. In a preferred embodiment, ubiquitin is provided in a reaction buffer solution, followed by addition of the ubiquitination enzymes. In an alternate preferred embodiment, ubiquitin is provided in a reaction buffer solution, a candidate modulator is then added, followed by addition of the ubiquitination enzymes.

In general, a test agent that decreases a PEM-3-like ubiquitin-related activity may be used to inhibit PEM-3-like protein function in vivo, while a test agent that increases a PEM-3-like ubiquitin-related activity may be used to stimulate PEM-3-like function in vivo. Test agent may be modified for use in vivo, e.g., by addition of a hydrophobic moiety, such as an ester.

Certain embodiments of the invention relate to assays for identifying agents that bind to a PEM-3-like polypeptide, optionally a particular domain of a PEM-3-like protein such as a KH domain or a RING domain. A wide variety of assays may be used for this purpose, including labeled in vitro protein-protein binding assays, electrophoretic mobility shift assays, immunoassays for protein binding, and the like. The purified protein may also be used for determination of three-dimensional crystal structure, which can be used for modeling intermolecular interactions and design of test agents. In one embodiment, an assay detects agents which inhibit interaction of one or more subject PEM-3-like polypeptides with a PEM-3-like-AP. In another embodiment, the assay detects agents which modulate the intrinsic biological activity of a PEM-3-like polypeptide or PEM-3-like protein complex, such as an enzymatic activity, binding to other cellular components, cellular compartmentalization, and the like.

In one aspect, the invention provides methods and compositions for the identification of compositions that interfere with the function of PEM-3-like polypeptides. Given the role of PEM-3-like polypeptides in viral production, compositions that perturb the formation or stability of the protein-protein interactions between PEM-3-like polypeptides and the proteins that they interact with, such as PEM-3-like-APs, and particularly PEM-3-like protein complexes comprising a viral protein, are candidate pharmaceuticals for the treatment of viral infections.

While not wishing to be bound to mechanism, it is postulated that PEM-3-like polypeptides promote the assembly of protein complexes that are important in release of virions. Complexes of the invention may include a combination of a PEM-3-like polypeptide and one or more of the following: a PEM-3-like-AP; a PEM-3-like polypeptide (as in the case of a PEM-3-like dimer, a heterodimer of two different PEM-3-like, homomultimers and heteromultimers); a Gag, particularly an HIV Gag; an E2 enzyme; a cullin; a clathrin; AP-1; AP-2; as well as, in certain embodiments, proteins known to be associated with clathrin-coated vesicles and or proteins involved in the protein sorting pathway.

The type of complex formed by a PEM-3-like polypeptide will depend upon the domains present in the protein. While not intended to be limiting, exemplary domains of potential interacting proteins are provided below. A RING domain is expected to interact with cullin, E2 enzymes, AP-1, AP-2, and/or a substrate for ubiquitynation (e.g., protein comprising a Gag L domain).

In a preferred assay for an antiviral agent, the test agent is assessed for its ability to disrupt or inhibit formation of a complex of a PEM-3-like polypeptide and a Gag polypeptide (especially a polypeptide comprising an HIV L domain).

A variety of assay formats will suffice and, in light of the present disclosure, those not expressly described herein will nevertheless be comprehended by one of ordinary skill in the art. Assay formats which approximate such conditions as formation of protein complexes, enzymatic activity, and even a PEM-3-like polypeptide-mediated membrane reorganization or vesicle formation activity, may be generated in many different forms, and include assays based on cell-free systems, e.g., purified proteins or cell lysates, as well as cell-based assays which utilize intact cells. Simple binding assays can also be used to detect agents which bind to PEM-3-like protein. Such binding assays may also identify agents that act by disrupting the interaction between a PEM-3-like polypeptide and a PEM-3-like interacting protein, or the transfer of ubiquitin to a PEM-3-like-AP or by disrupting the binding of a PEM-3-like polypeptide or complex to a substrate. Agents to be tested can be produced, for example, by bacteria, yeast or other organisms (e.g., natural products), produced chemically (e.g., small molecules, including peptidomimetics), or produced recombinantly. In a preferred embodiment, the test agent is a small organic molecule, e.g., other than a peptide or oligonucleotide, having a molecular weight of less than about 2,000 daltons.

In many drug screening programs which test libraries of compounds and natural extracts, high throughput assays are desirable in order to maximize the number of compounds surveyed in a given period of time. Assays of the present invention which are performed in cell-free systems, such as may be developed with purified or semi-purified proteins or with lysates, are often preferred as “primary” screens in that they can be generated to permit rapid development and relatively easy detection of an alteration in a molecular target which is mediated by a test compound. Moreover, the effects of cellular toxicity and/or bioavailability of the test compound can be generally ignored in the in vitro system, the assay instead being focused primarily on the effect of the drug on the molecular target as may be manifest in an alteration of binding affinity with other proteins or changes in enzymatic properties of the molecular target.

In preferred in vitro embodiments of the present assay, a reconstituted PEM-3-like protein complex comprises a reconstituted mixture of at least semi-purified proteins. By semi-purified, it is meant that the proteins utilized in the reconstituted mixture have been previously separated from other cellular or viral proteins. For instance, in contrast to cell lysates, the proteins involved in PEM-3-like protein complex formation are present in the mixture to at least 50% purity relative to all other proteins in the mixture, and more preferably are present at 90-95% purity. In certain embodiments of the subject method, the reconstituted protein mixture is derived by mixing highly purified proteins such that the reconstituted mixture substantially lacks other proteins (such as of cellular or viral origin) which might interfere with or otherwise alter the ability to measure PEM-3-like protein complex assembly and/or disassembly.

Assaying PEM-3-like protein complexes, in the presence and absence of a candidate inhibitor, can be accomplished in any vessel suitable for containing the reactants. Examples include microtitre plates, test tubes, and micro-centrifuge tubes.

In one embodiment of the present invention, drug screening assays can be generated which detect inhibitory agents on the basis of their ability to interfere with assembly or stability of the PEM-3-like protein complex. In an exemplary binding assay, the compound of interest is contacted with a mixture comprising a PEM-3-like polypeptide and at least one interacting polypeptide. Detection and quantification of PEM-3-like protein complexes provides a means for determining the compound's efficacy at inhibiting (or potentiating) interaction between the two polypeptides. The efficacy of the compound can be assessed by generating dose response curves from data obtained using various concentrations of the test compound. Moreover, a control assay can also be performed to provide a baseline for comparison. In the control assay, the formation of complexes is quantitated in the absence of the test compound.

Complex formation between the PEM-3-like polypeptides and a substrate polypeptide may be detected by a variety of techniques, many of which are effectively described above. For instance, modulation in the formation of complexes can be quantitated using, for example, delectably labeled proteins (e.g., radiolabeled, fluorescently labeled, or enzymatically labeled), by immunoassay, or by chromatographic detection. Surface plasmon resonance systems, such as those available from Biacore International AB (Uppsala, Sweden), may also be used to detect protein-protein interaction Often, it will be desirable to immobilize one of the polypeptides to facilitate separation of complexes from uncomplexed forms of one of the proteins, as well as to accommodate automation of the assay. In an illustrative embodiment, a fusion protein can be provided which adds a domain that permits the protein to be bound to an insoluble matrix. For example, GST-PEM-3-like fusion proteins can be adsorbed onto glutathione sepharose beads (Sigma Chemical, St. Louis, Mo.) or glutathione derivatized microtitre plates, which are then combined with a potential interacting protein, e.g., an 35S-labeled polypeptide, and the test compound and incubated under conditions conducive to complex formation. Following incubation, the beads are washed to remove any unbound interacting protein, and the matrix bead-bound radiolabel determined directly (e.g., beads placed in scintillant), or in the supernatant after the complexes are dissociated, e.g., when microtitre plate is used. Alternatively, after washing away unbound protein, the complexes can be dissociated from the matrix, separated by SDS-PAGE gel, and the level of interacting polypeptide found in the matrix-bound fraction quantitated from the gel using standard electrophoretic techniques.

In a further embodiment, agents that bind to a PEM-3-like polypeptide may be identified by using an immobilized PEM-3-like polypeptide. In an illustrative embodiment, a fusion protein can be provided which adds a domain that permits the protein to be bound to an insoluble matrix. For example, GST-PEM-3-like fusion proteins can be adsorbed onto glutathione sepharose beads (Sigma Chemical, St. Louis, Mo.) or glutathione derivatized microtitre plates, which are then combined with a potential labeled binding agent and incubated under conditions conducive to binding. Following incubation, the beads are washed to remove any unbound agent, and the matrix bead-bound label determined directly, or in the supernatant after the bound agent is dissociated.

In yet another embodiment, the PEM-3-like polypeptide and potential interacting polypeptide can be used to generate an interaction trap assay (see also, U.S. Pat. No. 5,283,317; Zervos et al. (1993) Cell 72:223-232; Madura et al. (1993) J Biol Chem 268:12046-12054; Bartel et al. (1993) Biotechniques 14:920-924; and Iwabuchi et al. (1993) Oncogene 8:1693-1696), for subsequently detecting agents which disrupt binding of the proteins to one and other.

In particular, the method makes use of chimeric genes which express hybrid proteins. To illustrate, a first hybrid gene comprises the coding sequence for a DNA-binding domain of a transcriptional activator can be fused in frame to the coding sequence for a “Ibait” protein, e.g., a PEM-3-like polypeptide of sufficient length to bind to a potential interacting protein. The second hybrid protein encodes a transcriptional activation domain fused in frame to a gene encoding a “fish” protein, e.g., a potential interacting protein of sufficient length to interact with the PEM-3-like polypeptide portion of the bait fusion protein. If the bait and fish proteins are able to interact, e.g., form a PEM-3-like protein complex, they bring into close proximity the two domains of the transcriptional activator. This proximity causes transcription of a reporter gene which is operably linked to a transcriptional regulatory site responsive to the transcriptional activator, and expression of the reporter gene can be detected and used to score for the interaction of the bait and fish proteins.

In accordance with the present invention, the method includes providing a host cell, preferably a yeast cell, e.g., Kluyverei lactis, Schizosaccharomyces pombe, Ustilago maydis, Saccharomyces cerevisiae, Neurospora crassa, Aspergillus niger, Aspergillus nidulans, Pichia pastoris, Candida tropicalis, and Hansenula polymorpha, though most preferably S cerevisiae or S. pombe. The host cell contains a reporter gene having a binding site for the DNA-binding domain of a transcriptional activator used in the bait protein, such that the reporter gene expresses a detectable gene product when the gene is transcriptionally activated. The first chimeric gene may be present in a chromosome of the host cell, or as part of an expression vector. Interaction trap assays may also be performed in mammalian and bacterial cell types.

The host cell also contains a first chimeric gene which is capable of being expressed in the host cell. The gene encodes a chimeric protein, which comprises (i) a DNA-binding domain that recognizes the responsive element on the reporter gene in the host cell, and (ii) a bait protein, such as a PEM-3-like polypeptide sequence.

A second chimeric gene is also provided which is capable of being expressed in the host cell, and encodes the “fish” fusion protein. In one embodiment, both the first and the second chimeric genes are introduced into the host cell in the form of plasmids. Preferably, however, the first chimeric gene is present in a chromosome of the host cell and the second chimeric gene is introduced into the host cell as part of a plasmid.

Preferably, the DNA-binding domain of the first hybrid protein and the transcriptional activation domain of the second hybrid protein are derived from transcriptional activators having separable DNA-binding and transcriptional activation domains. For instance, these separate DNA-binding and transcriptional activation domains are known to be found in the yeast GAL4 protein, and are known to be found in the yeast GCN4 and ADR1 proteins. Many other proteins involved in transcription also have separable binding and transcriptional activation domains which make them useful for the present invention, and include, for example, the LexA and VP16 proteins. It will be understood that other (substantially) transcriptionally-inert DNA-binding domains may be used in the subject constructs; such as domains of ACE1, 1cI, lac repressor, jun or fos. In another embodiment, the DNA-binding domain and the transcriptional activation domain may be from different proteins. The use of a LexA DNA binding domain provides certain advantages. For example, in yeast, the LexA moiety contains no activation function and has no known effect on transcription of yeast genes. In addition, use of LexA allows control over the sensitivity of the assay to the level of interaction (see, for example, the Brent et al. PCT publication WO94/10300).

In preferred embodiments, any enzymatic activity associated with the bait or fish proteins is inactivated, e.g., dominant negative or other mutants of a PEM-3-like polypeptide can be used.

Continuing with the illustrated example, the PEM-3-like polypeptide-mediated interaction, if any, between the bait and fish fusion proteins in the host cell, therefore, causes the activation domain to activate transcription of the reporter gene. The method is carried out by introducing the first chimeric gene and the second chimeric gene into the host cell, and subjecting that cell to conditions under which the bait and fish fusion proteins and are expressed in sufficient quantity for the reporter gene to be activated. The formation of a PEM-3-like-PEM-3-like-AP complex results in a detectable signal produced by the expression of the reporter gene. Accordingly, the level of formation of a complex in the presence of a test compound and in the absence of the test compound can be evaluated by detecting the level of expression of the reporter gene in each case. Various reporter constructs may be used in accord with the methods of the invention and include, for example, reporter genes which produce such detectable signals as selected from the group consisting of an enzymatic signal, a fluorescent signal, a phosphorescent signal and drug resistance.

One aspect of the present invention provides reconstituted protein preparations including a PEM-3-like polypeptide and one or more interacting polypeptides.

In still further embodiments of the present assay, the PEM-3-like protein complex is generated in whole cells, taking advantage of cell culture techniques to support the subject assay. For example, as described below, the PEM-3-like protein complex can be constituted in a eukaryotic cell culture system, including mammalian and yeast cells. Often it will be desirable to express one or more viral proteins (e.g., Gag or Env) in such a cell along with a subject PEM-3-like polypeptide. It may also be desirable to infect the cell with a virus of interest. Advantages to generating the subject assay in an intact cell include the ability to detect inhibitors which are functional in an environment more closely approximating that which therapeutic use of the inhibitor would require, including the ability of the agent to gain entry into the cell. Furthermore, certain of the in vivo embodiments of the assay, such as examples given below, are amenable to high through-put analysis of candidate agents.

The components of the PEM-3-like protein complex can be endogenous to the cell selected to support the assay. Alternatively, some or all of the components can be derived from exogenous sources. For instance, fusion proteins can be introduced into the cell by recombinant techniques (such as through the use of an expression vector), as well as by microinjecting the fusion protein itself or mRNA encoding the fusion protein.

In many embodiments, a cell is manipulated after incubation with a candidate agent and assayed for a PEM-3-like protein activity. In certain embodiments a PEM-3-like protein activity is represented by production of virus like particles. As demonstrated herein, an agent that disrupts PEM-3-like protein activity can cause a decrease in the production of virus like particles. In certain embodiments, PEM-3-like protein activities may include, without limitation, complex formation, ubiquitination and membrane fusion events (e.g., release of viral buds or fusion of vesicles). PEM-3-like protein complex formation may be assessed by immunoprecipitation and analysis of co-immunoprecipiated proteins or affinity purification and analysis of co-purified proteins. Fluorescence Resonance Energy Transfer (FRET)-based assays may also be used to determine complex formation. Fluorescent molecules having the proper emission and excitation spectra that are brought into close proximity with one another can exhibit FRET. The fluorescent molecules are chosen such that the emission spectrum of one of the molecules (the donor molecule) overlaps with the excitation spectrum of the other molecule (the acceptor molecule). The donor molecule is excited by light of appropriate intensity within the donor's excitation spectrum. The donor then emits the absorbed energy as fluorescent light. The fluorescent energy it produces is quenched by the acceptor molecule. FRET can be manifested as a reduction in the intensity of the fluorescent signal from the donor, reduction in the lifetime of its excited state, and/or re-emission of fluorescent light at the longer wavelengths (lower energies) characteristic of the acceptor. When the fluorescent proteins physically separate, FRET effects are diminished or eliminated. (U.S. Pat. No. 5,981,200).

For example, a cyan fluorescent protein is excited by light at roughly 425-450 nm wavelength and emits light in the range of 450-500 nm. Yellow fluorescent protein is excited by light at roughly 500-525 nm and emits light at 525-500 nm. If these two proteins are placed in solution, the cyan and yellow fluorescence may be separately visualized. However, if these two proteins are forced into close proximity with each other, the fluorescent properties will be altered by FRET. The bluish light emitted by CFP will be absorbed by YFP and re-emitted as yellow light. This means that when the proteins are stimulated with light at wavelength 450 nm, the cyan emitted light is greatly reduced and the yellow light, which is not normally stimulated at this wavelength, is greatly increased. FRET is typically monitored by measuring the spectrum of emitted light in response to stimulation with light in the excitation range of the donor and calculating a ratio between the donor-emitted light and the acceptor-emitted light. When the donor:acceptor emission ratio is high, FRET is not occurring and the two fluorescent proteins are not in close proximity. When the donor: acceptor emission ratio is low, FRET is occurring and the two fluorescent proteins are in close proximity. In this manner, the interaction between a first and second polypeptide may be measured.

The occurrence of FRET also causes the fluorescence lifetime of the donor fluorescent moiety to decrease. This change in fluorescence lifetime can be measured using a technique termed fluorescence lifetime imaging technology (FLM) (Verveer et al. (2000) Science 290: 1567-1570; Squire et al. (1999) J. Microsc. 193: 36; Verveer et al. (2000) Biophys. J. 78: 2127). Global analysis techniques for analyzing FLIM data have been developed. These algorithms use the understanding that the donor fluorescent moiety exists in only a limited number of states each with a distinct fluorescence lifetime. Quantitative maps of each state can be generated on a pixel-by-pixel basis.

To perform FRET-based assays, the PEM-3-like polypeptide and the interacting protein of interest are both fluorescently labeled. Suitable fluorescent labels are, in view of this specification, well known in the art. Examples are provided below, but suitable fluorescent labels not specifically discussed are also available to those of skill in the art. Fluorescent labeling may be accomplished by expressing a polypeptide as a fusion protein with a fluorescent protein, for example fluorescent proteins isolated from jellyfish, corals and other coelenterates. Exemplary fluorescent proteins include the many variants of the green fluorescent protein (GFP) of Aequoria victoria. Variants may be brighter, dimmer, or have different excitation and/or emission spectra. Certain variants are altered such that they no longer appear green, and may appear blue, cyan, yellow or red (termed BFP, CFP, YFP and RFP, respectively). Fluorescent proteins may be stably attached to polypeptides through a variety of covalent and noncovalent linkages, including, for example, peptide bonds (e.g., expression as a fusion protein), chemical cross linking and biotin-streptavidin coupling. For examples of fluorescent proteins, see U.S. Pat. Nos. 5,625,048; 5,777,079; 6,066,476; 6,124,128; Prasher et al. (1992) Gene, 111:229-233; Heim et al. (1994) Proc. Natl. Acad. Sci., USA, 91:12501-04; Ward et al. (1982) Photochem. Photobiol., 35:803-808 ; Levine et al. (1982) Comp. Biochem. Physiol., 72B:77-85; Tersikh et al. (2000) Science 290: 1585-88.

Other exemplary fluorescent moieties well known in the art include derivatives of fluorescein, benzoxadioazole, coumarin, eosin, Lucifer Yellow, pyridyloxazole and rhodamine. These and many other exemplary fluorescent moieties may be found in the Handbook of Fluorescent Probes and Research Chemicals (2000, Molecular Probes, Inc.), along with methodologies for modifying polypeptides with such moieties. Exemplary proteins that fluoresce when combined with a fluorescent moiety include, yellow fluorescent protein from Vibrio fischeri Baldwin et al. (1990) Biochemistry 29:5509-15), peridinin-chlorophyll a binding protein from the dinoflagellate Symbiodiniuin sp. (Morris et al. (1994) Plant Molecular Biology 24:673:77) and phycobiliproteins from marine cyanobacteria such as Synechococcus, e.g., phycoerythrin and phycocyanin (Wilbanks et al. (1993) J. Biol. Chem. 268:1226-35). These proteins require flavins, peridinin-chlorophyll a and various phycobilins, respectively, as fluorescent co-factors.

FRET-based assays may be used in cell-based assays and in cell-free assays. FRET-based assays are amenable to high-throughput screening methods including Fluorescence Activated Cell Sorting and fluorescent scanning of microtiter arrays.

In a further embodiment, transcript levels may be measured in cells having higher or lower levels of PEM-3-like protein activity in order to identify genes that are regulated by PEM-3-like protein. Promoter regions for such genes (or larger portions of such genes) may be operatively linked to a reporter gene and used in a reporter gene-based assay to detect agents that enhance or diminish PEM-3-like-regulated gene expression. Transcript levels may be determined in any way known in the art, such as, for example, Northern blotting, RT-PCR, microarray, etc. Increased PEM-3-like protein activity may be achieved, for example, by introducing a strong PEM-3-like expression vector. Decreased PEM-3-like protein activity may be achieved, for example, by RNAi, antisense, ribozyme, gene knockout, etc.

In general, where the screening assay is a binding assay (whether protein-protein binding, agent-protein binding, etc.), one or more of the molecules may be joined to a label, where the label can directly or indirectly provide a detectable signal. Various labels include radioisotopes, fluorescers, chemiluminescers, enzymes, specific binding molecules, particles, e.g., magnetic particles, and the like. Specific binding molecules include pairs, such as biotin and streptavidin, digoxin and antidigoxin etc. For the specific binding members, the complementary member would normally be labeled with a molecule that provides for detection, in accordance with known procedures.

A variety of other reagents may be included in the screening assay. These include reagents like salts, neutral proteins, e.g., albumin, detergents, etc that are used to facilitate optimal protein-protein binding and/or reduce nonspecific or background interactions. Reagents that improve the efficiency of the assay, such as protease inhibitors, nuclease inhibitors, anti- microbial agents, etc. may be used. The mixture of components are added in any order that provides for the requisite binding. Incubations are performed at any suitable temperature, typically between 4° and 40° C. Incubation periods are selected for optimum activity, but may also be optimized to facilitate rapid high-throughput screening.

In, certain embodiments, a test agent may be assessed for its ability to perturb the localization of a PEM-3-like polypeptide, e.g., preventing PEM-3-like polypeptide localization to the nucleus.

10. Methods and Compositions for Treatment of Viral Disorders

In a further aspect, the invention provides methods and compositions for treatment of viral disorders, and particularly disorders caused by envelop viruses, retroid viruses and RNA viruses, including but not limited to retroviruses, rhabdoviruses, lentiviruses, and filoviruses. Preferred therapeutics of the invention function by disrupting the biological activity of a PEM-3-like polypeptide or PEM-3-like protein complex in viral maturation.

Exemplary therapeutics of the invention include nucleic acid therapies such as for example RNAi constructs, antisense oligonucleotides, ribozyme, and DNA enzymes. Other PEM-3-like protein therapeutics include polypeptides, peptidomimetics, antibodies and small molecules.

Antisense therapies of the invention include methods of introducing antisense nucleic acids to disrupt the expression of PEM-3-like polypeptides or proteins that are necessary for PEM-3-like protein function.

RNAi therapies include methods of introducing RNAi constructs to downregulate the expression of PEM-3-like polypeptides or proteins that are necessary for PEM-3-like protein function. An exemplary RNAi target sequence is depicted in SEQ ID NO: 21.

Therapeutic polypeptides may be generated by designing polypeptides to mimic certain protein domains important in the formation of PEM-3-like protein complexes, such as, for example KH domains or RING domains. In one embodiment, a binding partner may be Gag. In a further embodiment, a polypeptide that resembles an L domain may disrupt recruitment of Gag to the PEM-3-like protein complex.

In view of the specification, methods for generating antibodies directed to epitopes of PEM-3-like proteins and PEM-3-like-interacting proteins are known in the art. Antibodies may be introduced into cells by a variety of methods. One exemplary method comprises generating a nucleic acid encoding a single chain antibody that is capable of disrupting a PEM-3-like protein complex. Such a nucleic acid may be conjugated to antibody that binds to receptors on the surface of target cells. It is contemplated that in certain embodiments, the antibody may target viral proteins that are present on the surface of infected cells, and in this way deliver the nucleic acid only to infected cells. Once bound to the target cell surface, the antibody is taken up by endocytosis, and the conjugated nucleic acid is transcribed and translated to produce a single chain antibody that interacts with and disrupts the targeted PEM-3-like protein complex. Nucleic acids expressing the desired single chain antibody may also be introduced into cells using a variety of more conventional techniques, such as viral transfection (e.g., using an adenoviral system) or liposome-mediated transfection.

Small molecules of the invention may be identified for their ability to modulate the formation of PEM-3-like protein complexes, as described above.

In view of the teachings herein, one of skill in the art will understand that the methods and compositions of the invention are applicable to a wide range of viruses such as for example retroid viruses and RNA viruses. In a preferred embodiment, the present invention is applicable to retroid viruses. In a more preferred embodiment, the present invention is further applicable to retroviruses (retroviridae). In another more preferred embodiment, the present invention is applicable to lentivirus, including primate lentivirus group.

While not intended to be limiting, relevant retroviruses include: C-type retrovirus which causes lymphosarcoma in Northern Pike, the C-type retrovirus which infects mink, the caprine lentivirus which infects sheep, the Equine Infectious Anemia Virus (EIAV), the C-type retrovirus which infects pigs, the Avian Leukosis Sarcoma Virus (ALSV), the Feline Leukemia Virus (FeLV), the Feline Aids Virus, the Bovine Leukemia Virus (BLV), the Simian Leukemia Virus (SLV), the Simian Immuno-deficiency Virus (SIV), the Human T-cell Leukemia Virus type-I (HTLV-I), the Human T-cell Leukemia Virus type-II (HTLV-II), Human Immunodeficiency virus type-2 (HIV-2) and Human Immunodeficiency virus type-1 (HIV-1).

The method and compositions of the present invention are further applicable to RNA viruses, including ssRNA negative-strand viruses. In a preferred embodiment, the present invention is applicable to mononegavirales, including filoviruses. Filoviruses further include Ebola viruses and Marburg viruses.

Other RNA viruses include picornaviruses such as enterovirus, poliovirus, coxsackievirus and hepatitis A virus, the caliciviruses, including Norwalk-like viruses, the rhabdoviruses, including rabies virus, the togaviruses including alphaviruses, Semliki Forest virus, denguevirus, yellow fever virus and rubella virus, the orthomyxoviruses, including Type A, B, and C influenza viruses, the bunyaviruses, including the Rift Valley fever virus and the hantavirus, the filoviruses such as Ebola virus and Marburg virus, and the paramyxoviruses, including mumps virus and measles virus. Additional viruses that may be treated include herpes viruses.

11. Effective Dose

Toxicity and therapeutic efficacy of such compounds can be determined by standard pharmaceutical procedures in cell cultures or experimental animals, e.g., for determining The Ld₅₀ (The Dose Lethal To 50% Of The Population) And The Ed₅₀ (the dose therapeutically effective in 50% of the population). The dose ratio between toxic and therapeutic effects is the therapeutic index and it can be expressed as the ratio LD₅₀/ED₅₀. Compounds which exhibit large therapeutic induces are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

12. Formulation and Use

Pharmaceutical compositions for use in accordance with the present invention may be formulated in conventional manner using one or more physiologically acceptable carriers or excipients. Thus, the compounds and their physiologically acceptable salts and solvates may be formulated for administration by, for example, injection, inhalation or insufflation (either through the mouth or the nose) or oral, buccal, parenteral or rectal administration.

An exemplary composition of the invention comprises an RNAi mixed with a delivery system, such as a liposome system, and optionally including an acceptable excipient. In a preferred embodiment, the composition is formulated for topical administration for, e.g., herpes virus infections.

For such therapy, the compounds of the invention can be formulated for a variety of loads of administration, including systemic and topical or localized administration. Techniques and formulations generally may be found in Remmington's Pharmaceutical Sciences, Meade Publishing Co., Easton, Pa. For systemic administration, injection is preferred, including intramuscular, intravenous, intraperitoneal, and subcutaneous. For injection, the compounds of the invention can be formulated in liquid solutions, preferably in physiologically compatible buffers such as Hank's solution or Ringer's solution. In addition, the compounds may be formulated in solid form and redissolved or suspended immediately prior to use. Lyophilized forms are also included.

For oral administration, the pharmaceutical compositions may take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., ationd oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate.

Preparations for oral administration may be suitably formulated to give controlled release of the active compound. For buccal administration the compositions may take the form of tablets or lozenges formulated in conventional manner. For administration by inhalation, the compounds for use according to the present invention are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g., gelatin for use in an inhaler or insufflator may be formulated containing a powder mix of the compound and a suitable powder base such as lactose or starch.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

The compounds may also be formulated in rectal compositions such as suppositories or retention enemas, e.g., containing conventional suppository bases such as cocoa butter or other glycerides.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration bile salts and fusidic acid derivatives. In addition, detergents may be used to facilitate permeation. Transmucosal administration may be through nasal sprays or using suppositories. For topical administration, the oligomers of the invention are formulated into ointments, salves, gels, or creams as generally known in the art. A wash solution can be used locally to treat an injury or inflammation to accelerate healing.

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration.

For therapies involving the administration of nucleic acids, the oligomers of the invention can be formulated for a variety of modes of administration, including systemic and topical or localized administration. Techniques and formulations generally may be found in Remmington's Pharmaceutical Sciences, Meade Publishing Co., Easton, Pa. For systemic administration, injection is preferred, including intramuscular, intravenous, intraperitoneal, intranodal, and subcutaneous for injection, the oligomers of the invention can be formulated in liquid solutions, preferably in physiologically compatible buffers such as Hank's solution or Ringer's solution. In addition, the oligomers may be formulated in solid form and redissolved or suspended immediately prior to use. Lyophilized forms are also included.

Systemic administration can also be by transmucosal or transdermal means, or the compounds can be administered orally. For transmucosal or transdermal administration, penetrants appropriate to the barrier to be permeated are used in the formulation. Such penetrants are generally known in the art, and include, for example, for transmucosal administration bile salts and fusidic acid derivatives. In addition, detergents may be used to facilitate permeation. Transmucosal administration may be through nasal sprays or using suppositories. For oral administration, the oligomers are formulated into conventional oral administration forms such as capsules, tablets, and tonics. For topical administration, the oligomers of the invention are formulated into ointments, salves, gels, or creams as generally known in the art.

Exemplification

The invention now being generally described, it will be more readily understood by reference to the following examples, which are included merely for purposes of illustration of certain aspects and embodiments of the present invention, and are not intended to limit the invention.

### EXAMPLES

1. Involvement of PEM-3-Like Protein in HIV-1 gRNA Packaging

1. Day 1: plate 2×6-wells plate with HeLa-SS6 cells at 4.5×10⁵ cells/well (50% confluence on the next day).

2. Day 2-4: transfect as indicated in the table. (0.25 ml OptiMEM+5 μl Lipofectamine2000)+0.25 ml OptiMEM+DNA as indicated in the table). SiRNA: 187; scramble, 153; POSH, 193; PRT14-1, 225; PEM-3-like protein, 213; PRT15. Plasmids: #95; empty vector, #111-pNlenv-1, #387; mNC- pNlenv-1 (mutation in the nuclear capsid renders it unable to bind HIV RNA).

Transfections

| Day 4: | Transfection day 3; | | |
|---|---|---|---|
| VLP | 100 nM siRNA (12.5 ul) + | Transfection day 2 | |
| assay | 0.75 ug #111 or #387 or #95 | 100 nM siRNA (12.5 ul) | Well |
| | 187 + #111 | 187 | A1 |
| | 187 + #387 | 187 | A2 |
| | 153 + #111 | 153 | A3 |
| | 193 + #111 | 193 | A4 |
| | 225 + #111 | 225 | A5 |
| | 213 + #111 | 213 | A6 |
| | 187 + #95 | 187 | A7 |

Day 3: VLP assay
Steady state VLP assay

A. Cell extracts

1. Collect 2 ml medium and pellet floating cells by centrifugation (1 min, 14000 rpm at 4° C.), save sup (continue with sup immediately to step B), scrape cells in ice-cold PBS, add to the corresponding floated cell pellet and centrifuge for 5 min 1800 rpm at 4° C.

2. Wash cell pellet once with ice-cold PBS.

3. Resuspend cell pellet (from 6 well) in 100 μl NP40-DOC lysis buffer and incubate 10 minutes on ice.

4. Centrifuge at 14,000rpm for 15 min. Transfer supernatant to a clean eppendorf.

5. Prepare samples for SDS-PAGE by adding them sample buffer and boil for 10 min —take the same volume for each reaction (15 μl).

B. Purification of VLP From Cell Media

1. Filtrate the supernatant through a 0.45μ filter.

2. Centrifuge supernatant at 14,000 rpm at 4° C. for at least 2 h.

3. Resuspend VLP pellet of A1-A7 in 50 μl 1× sample buffer and boil for 10 min. Load 25 μl of each sample.

4. VLP pellets from B1-B7: continue to the Dot-blot assay.

C. Western Blot Analysis

1. Run all samples from stages A and B on Tris-Gly SDS-PAGE 12.5%.

2. Transfer samples to nitrocellulose membrane (100V for 1.15 h.).

3. Dye membrane with ponceau solution.

4. Block with 10% low fat milk in TBS-t for 1 h.

5. Incubate with anti p24 rabbit 1:500 in TBS-t 2 hour (room temperature)—o/n (4° C.).

6. Wash 3 times with TBS-t for 7 min each wash.

7. Incubate with secondary antibody anti rabbit cy5 1:500 for 30 min.

8. Wash five times for 10 min in TBS-t.

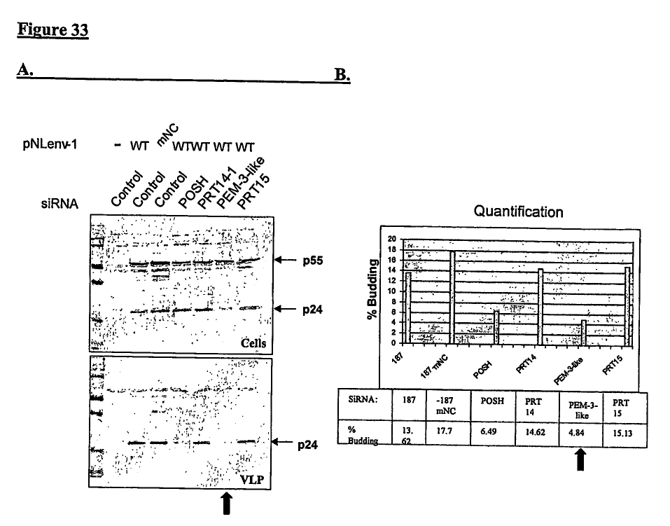

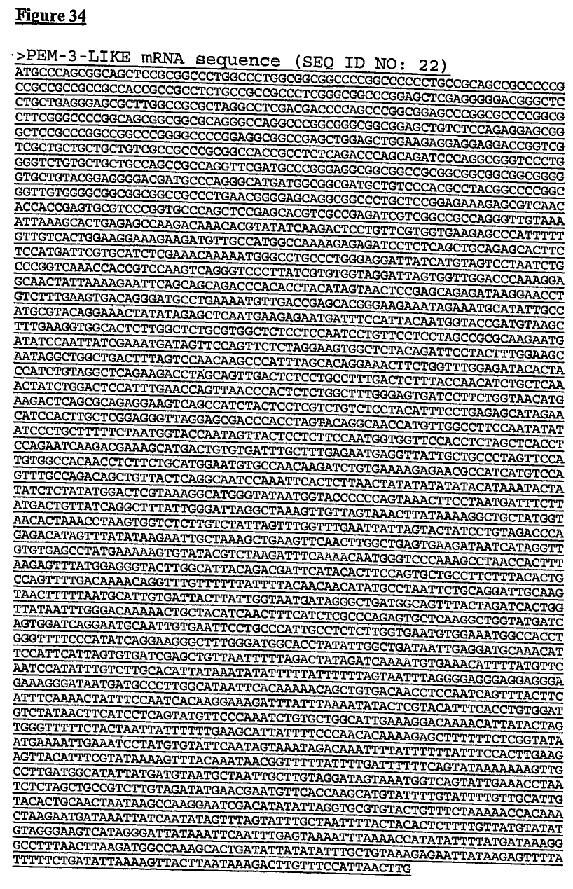

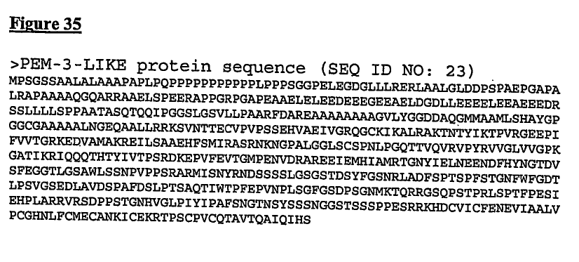

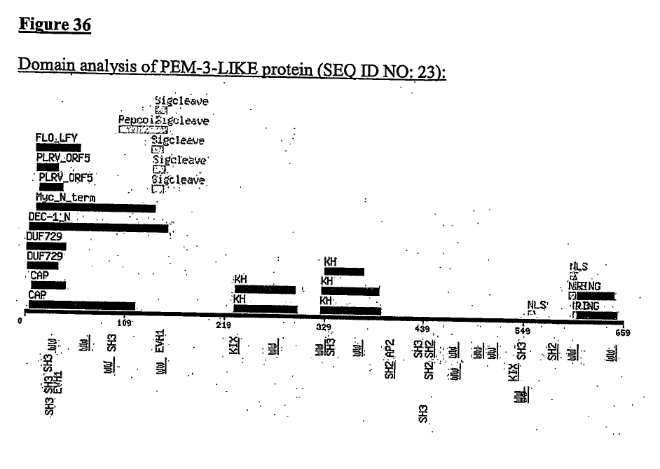

9. View in Typhoon for fluorescence signal (650). Results are depicted in FIG. 33.

Lysis buffer

| Tris-HCl pH 7.6 | 50 | mM |
|---|---|---|
| MgCl₂ | 1.5 | mM |
| NaCl | 150 | mM |
| Glycerol | 10% | |
| NP-40 | 0.5% | |
| DOC | 0.5% | |
| EDTA | 1 | mM |
| EGTA | 1 | mM |

Add PI₃C 1:200.

2. Exemplary siRNA Target Sequence

TAGDA-225:

(SEQ ID NO: 21)

PEM-3-like (117) AACCACCGTCCAAGTCAGGGT

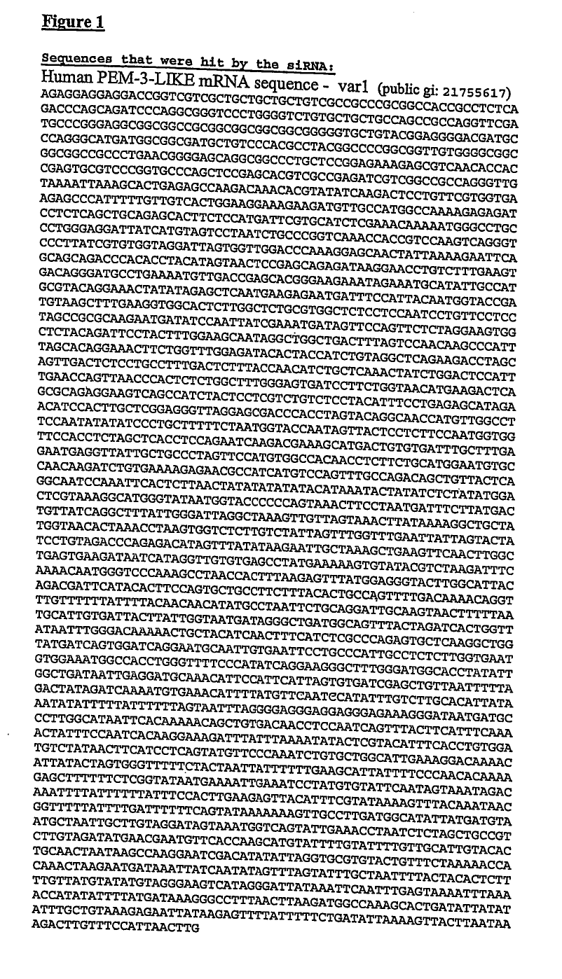

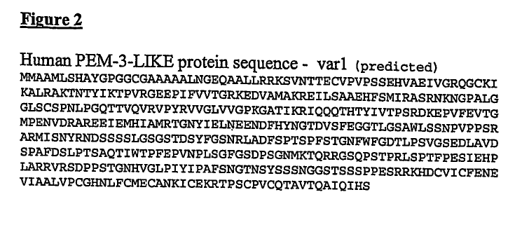

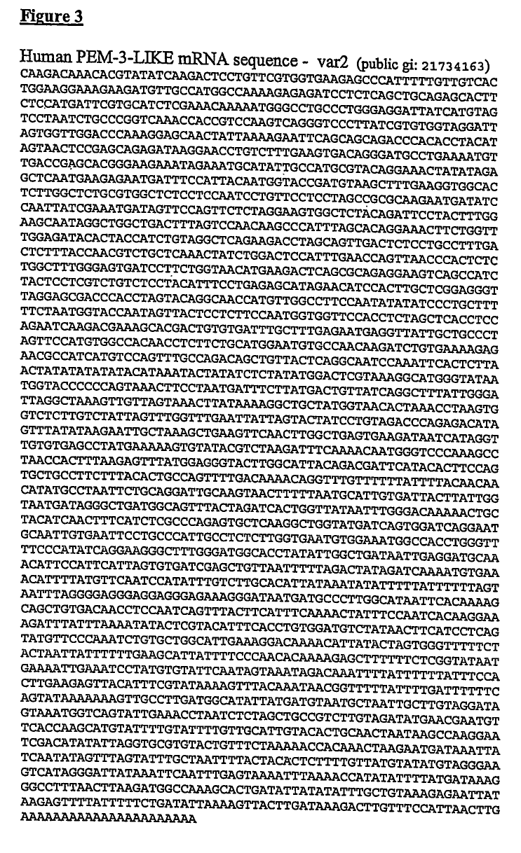

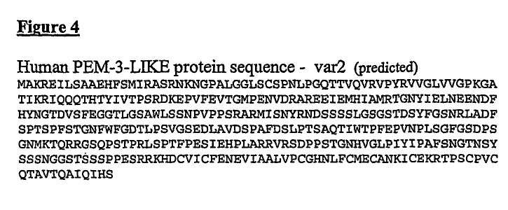

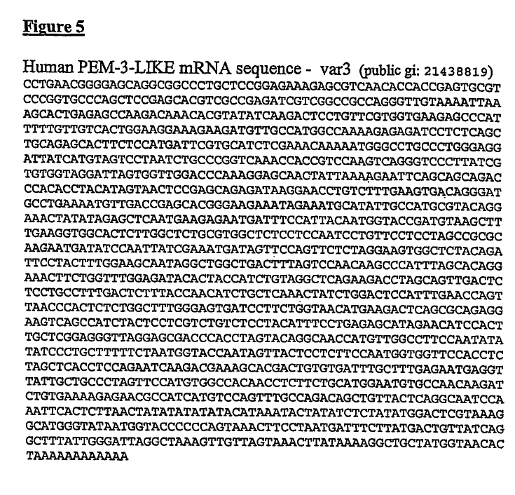

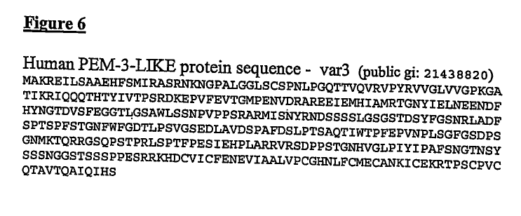

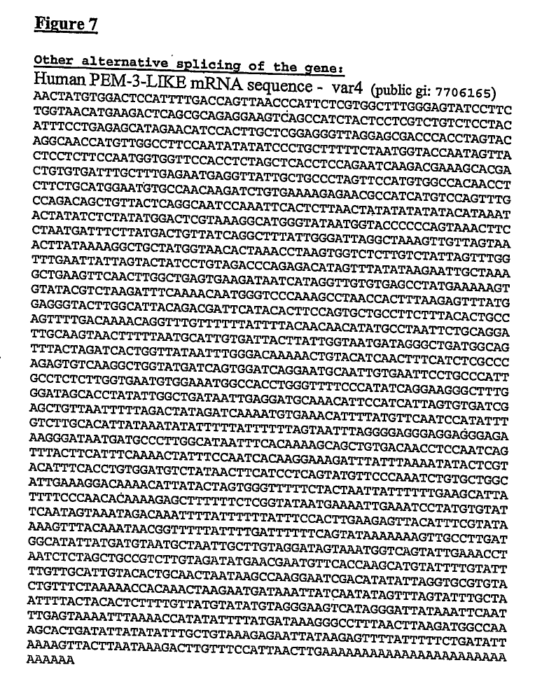

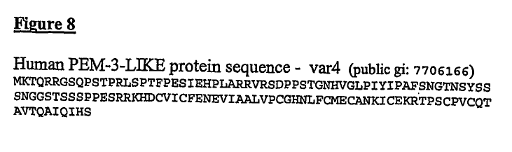

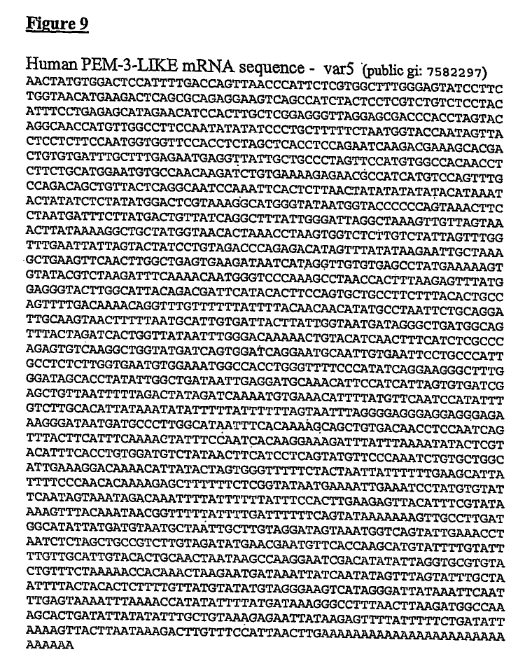

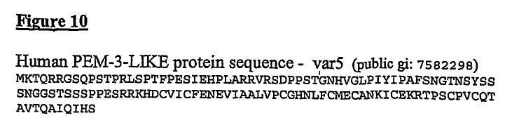

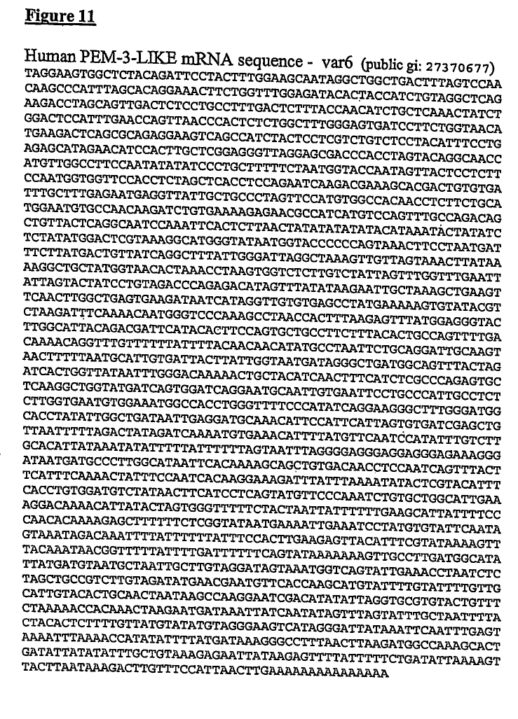

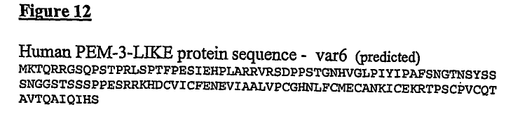

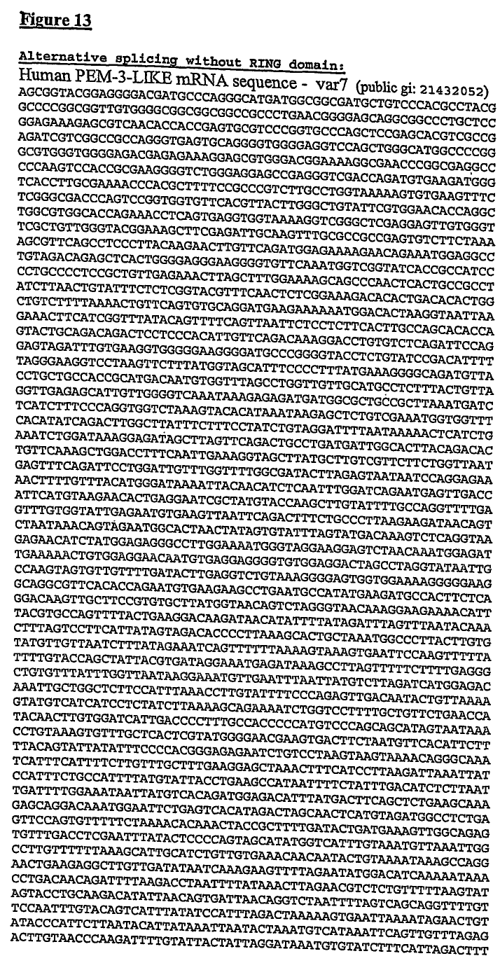

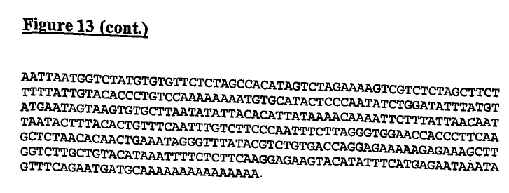

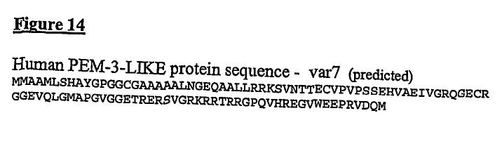

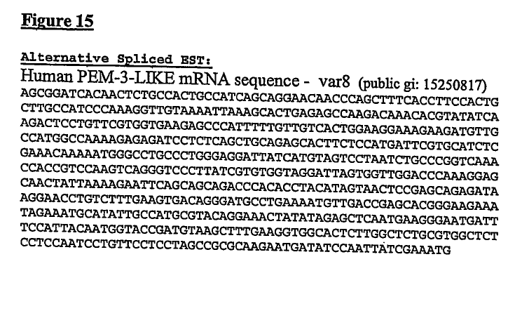

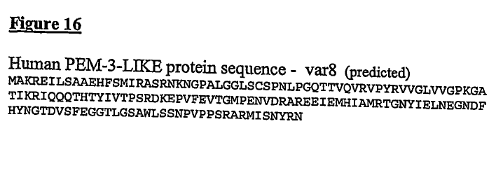

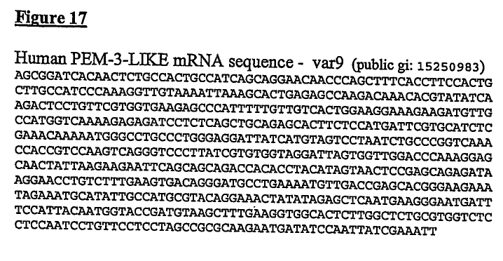

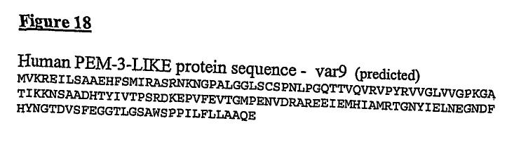

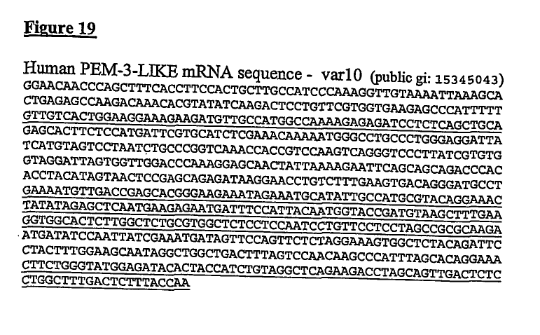

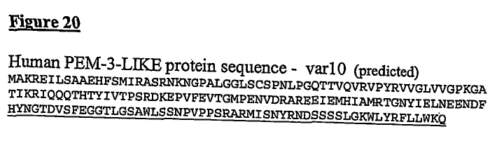

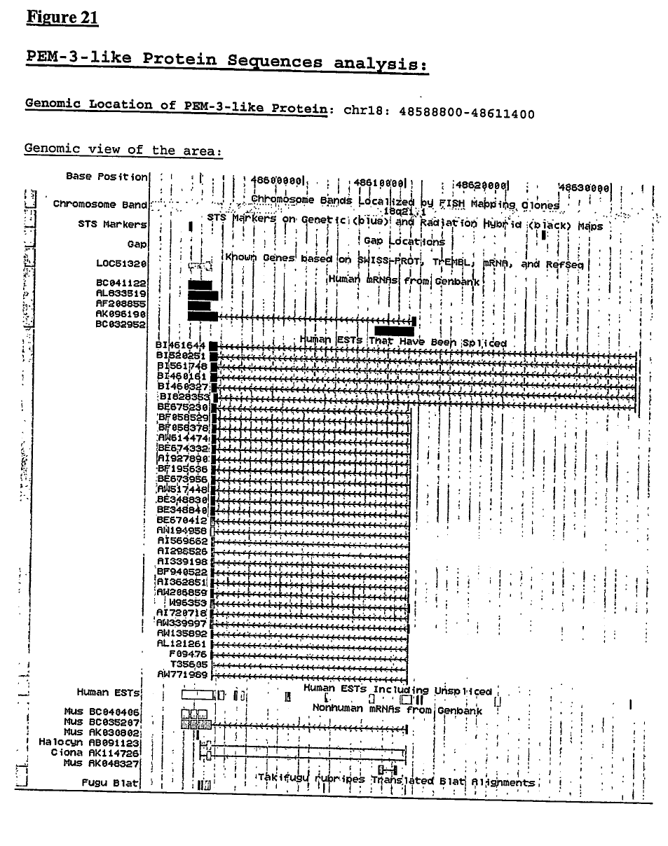

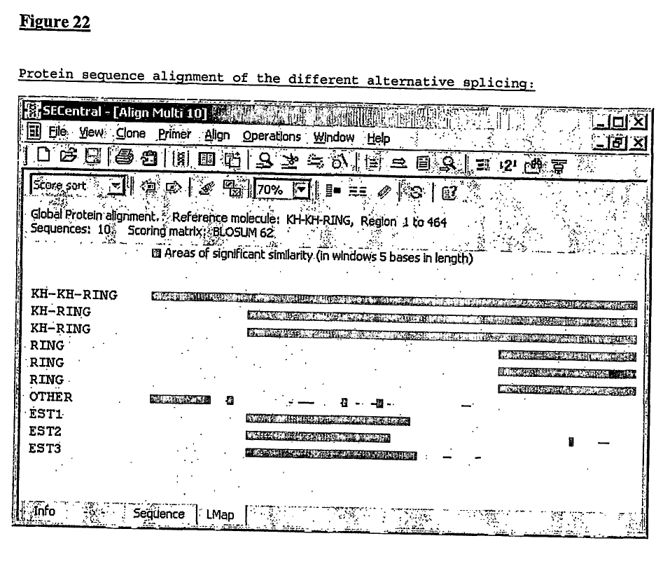

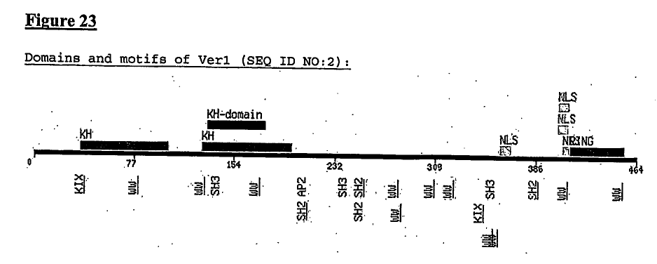

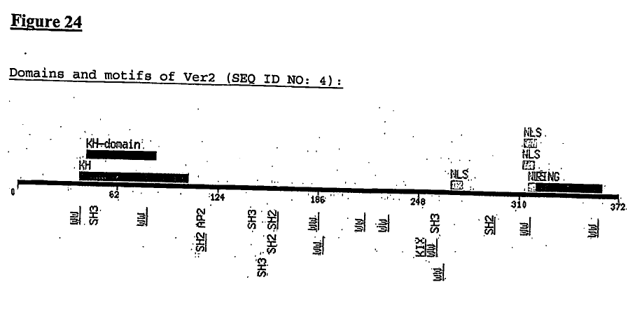

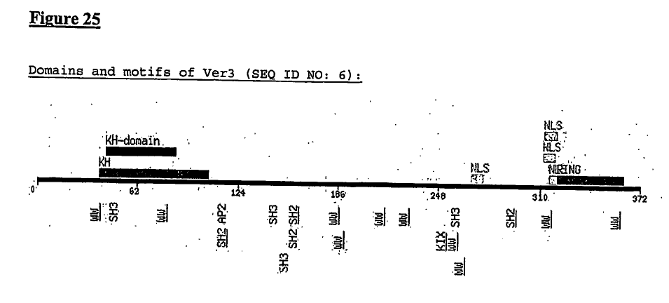

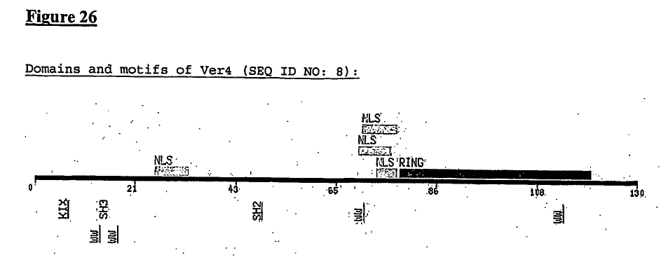

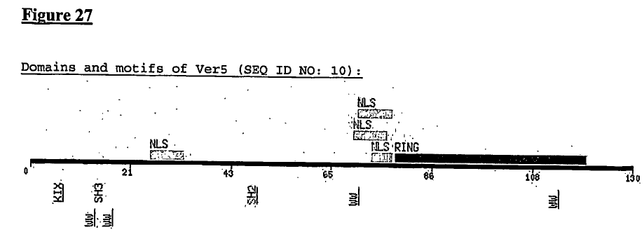

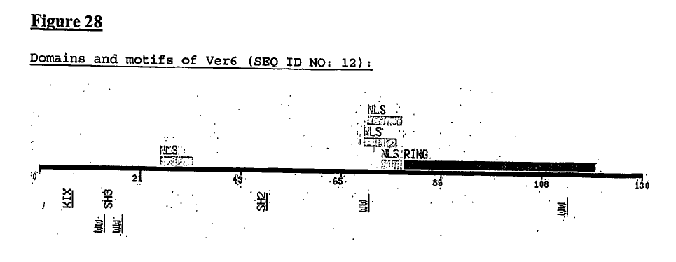

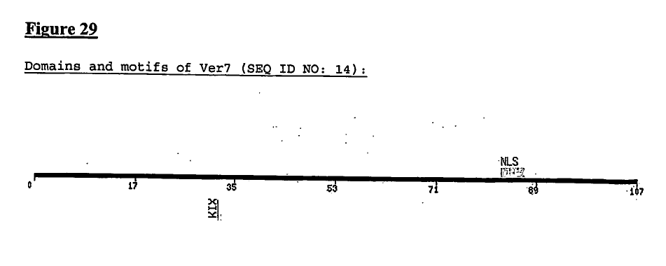

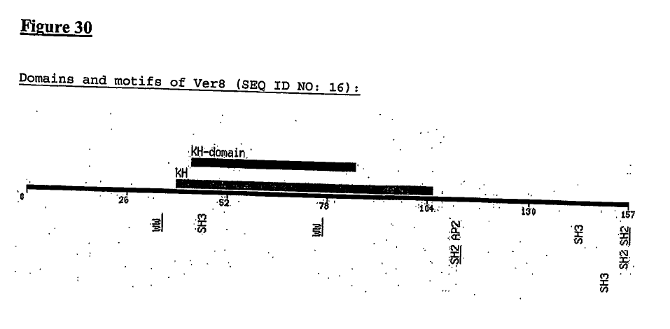

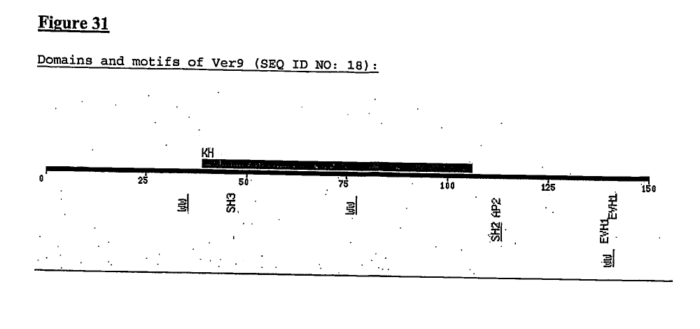

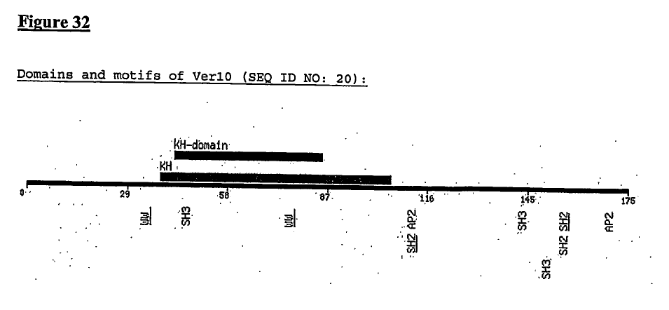

See FIGS. 1, 3, and 5 for examples of sequences that were hit by the siRNA.

3. PEM-3-Like Reduction Inhibits Viral Release and Infectivity

PEM-3-like reduction reduces reverse transcriptase (RT) activity in release virus-like-particles (VLP):

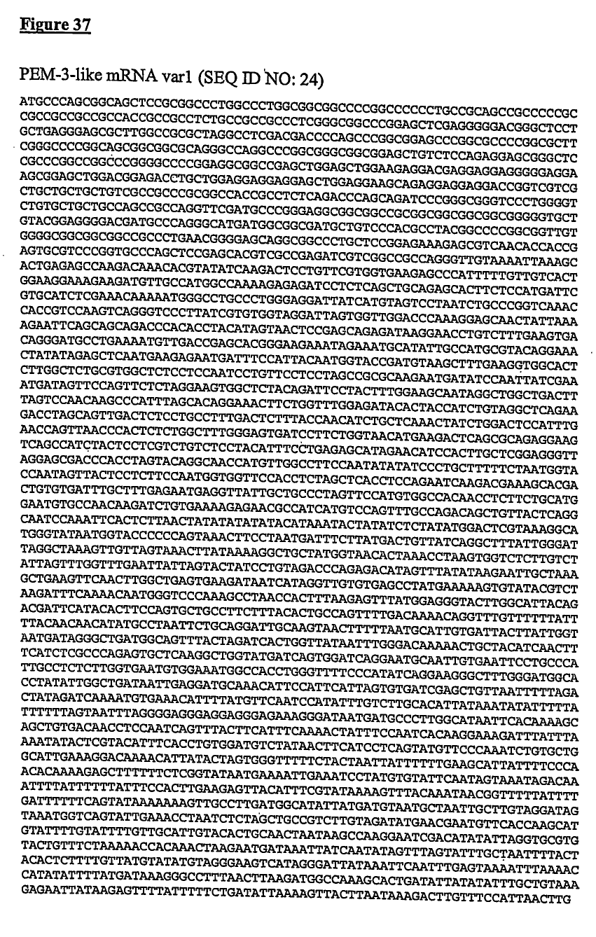

HeLa SS6 cell cultures (in triplicates) were transfected with siRNA targeting PEM-3-like or with a control siRNA. Following gene silencing by siRNA, cells were transfected with pNLenvl, encoding an envelope-deficient subviral Gag-Pol expression system (Schubert, U., Clouse, K. A., and Strebel, K. (1995). Augmentation of virus secretion by the human immunodeficiency virus type 1 Vpu protein is cell type independent and occurs in cultured human primary macrophages and lymphocytes. J Virol 69, 7699-7711) and RT activity in VLP released into the culture medium was determined (FIG. 37). Cells treated with PEM-3-like-specific siRNA reduced RT activity by 90 percent.

PEM-3-like protein acts upstream to virus budding at the cell surface:

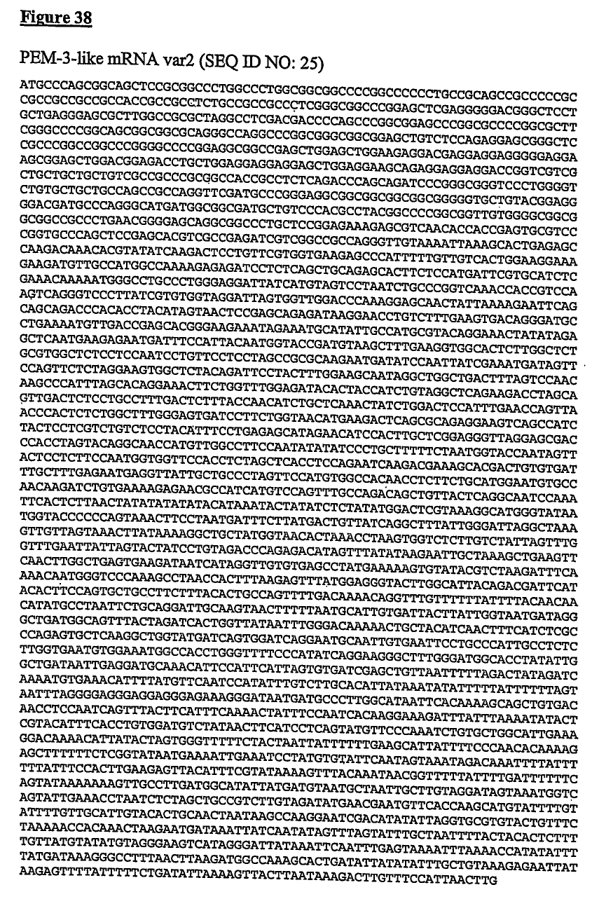

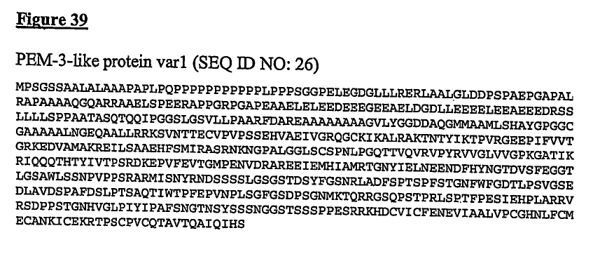

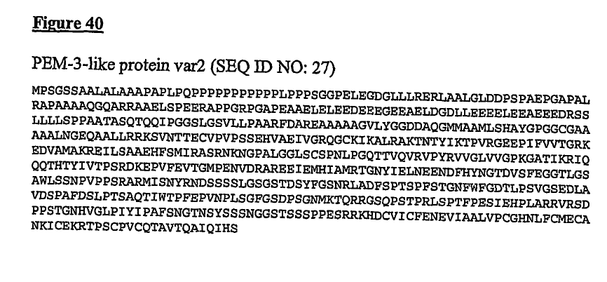

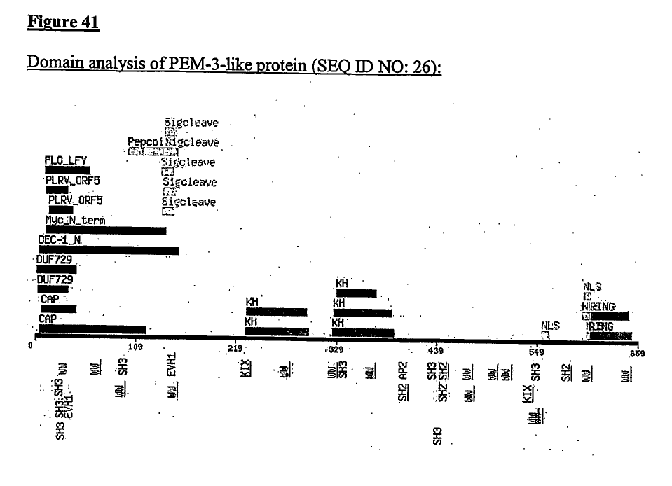

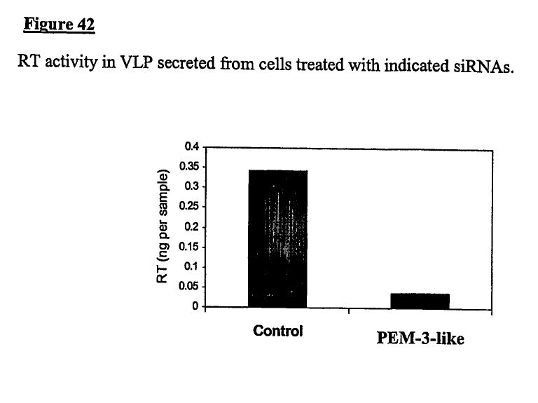

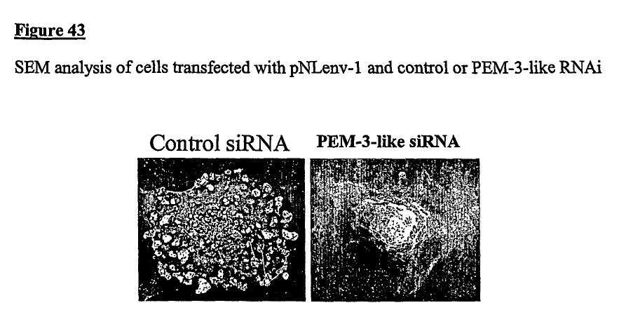

Scanning electron microscopy (SEM) revealed numerous cell surface-tethered virus particles, consistent with inhibition of virus release. Pre-treatment with PEM-3-like siRNA ablated virus budding, indicating that it functiones independently of the virus L-domain and upstream of virus budding at the cell membrane (FIG. 38 compare control and PEM-3-like RNAi).

Cell Culture and Transfections:

Hela SS6 cells were grown in Dulbecco's modified Eagle's medium (DMEM) supplemented with 10% heat-inactivated fetal calf serum and 100 units/ml penicillin and 100 μg/ml streptomycin. For transfections, HeLa SS6 cells were grown to 50% confluency in DMEM containing 10% FCS without antibiotics. Cells were then transfected with the relevant double-stranded siRNA (50-100 nM) using lipofectamin 2000 (Invitrogen, Paisley, UK). On the day following the initial transfection, cells were split 1:3 in complete medium and co-transfected 24 hours later with HIV-1NLenv1 (2 μg per 6-well) (Schubert, U., Clouse, K. A., and Strebel, K. (1995). Augmentation of virus secretion by the human immunodeficiency virus type 1 Vpu protein is cell type independent and occurs in cultured human primary macrophages and lymphocytes. J Virol 69, 7699-7711) and a second portion of double-stranded siRNA.

Assays for Virus Release by RT Activity:

Virus and virus-like particle (VLP) release was determined one day after transfection with the pro-viral DNA as previously described (Adachi, A., Gendelman, H. E., Koenig, S., Folks, T., Willey, R., Rabson, A., and Martin, M. A. (1986) Production of acquired immunodeficiency syndrome-associated retrovirus in human and nonhuman cells transfected with an infectious molecular clone. J Virol 59:284-291; Fukumori, T., Akari, H., Yoshida, A., Fujita, M., Koyama, A. H., Kagawa, S., and Adachi, A. (2000). Regulation of cell cycle and apoptosis by human immunodeficiency virus type 1 Vpr. Microbes Infect 2, 1011-1017; Lenardo, M. J., Angleman, S. B., Bounkeua, V., Dimas, J., Duvall, M. G., Graubard, M. B., Hornung, F., Selkirk, M. C., Speirs, C. K., Trageser, C., et al. (2002). Cytopathic killing of peripheral blood CD4(+) T lymphocytes by human immunodeficiency virus type 1 appears necrotic rather than apoptotic and does not require env. J Virol 76, 5082-5093). The culture medium of virus-expressing cells was collected and centrifuged at 500× g for 10 minutes. The resulting supernatant was passed through a 0.45 μm-pore filter and the filtrate was centrifuged at 14,000× g for 2 hours at 4° C. The resulting supernatant was removed and the viral-pellet was re-suspended in cell solubilization buffer (50 mM Tris-HCl, pH7.8, 80 mM potassium chloride, 0.75 mM EDTA and 0.5% Triton X-100, 2.5 mM DTT and protease inhibitors). The corresponding cells were washed three times with phosphate-buffered saline (PBS) and then solubilized by incubation on ice for 15 minutes in cell solubilization buffer. The cell detergent extract was then centrifuged for 15 minutes at 14,000× g at 4° C. The sample of the cleared extract (normally 1:10 of the initial sample) were resolved on a 12.5% SDS-polyacrylamide gel, then transferred onto nitrocellulose paper and subjected to immunoblot analysis with rabbit anti-CA antibodies. The CA was detected after incubation with a secondary anti-rabbit antibody conjugated to Cy5 (Jackson Laboratories, West Grove, Pa.) and detected by fluorescence imaging (Typhoon instrument, Molecular Dynamics, Sunnyvale, Calif.). The Pr55 and CA were then quantified by densitometry. A colorimetric reverse transcriptase assay (Roche Diagnostics GmbH, Mannenheim, Germany) was used to measure reverse transcriptase activity in VLP extracts. RT activity was normalized to amount of PrS5 and CA produced in the cells.

Scanning Electron Microscopy:

HeLa cells were fixed for two hours in 0.1M phosphate buffer (PB) (pH 7.2) containing 2.5% glutaraldehyde and then washed three times with PB. The cells were then dehydrated by gradual increase of the ethanol concentration (25%, 75%, 95%, 100%). The samples at 100% ethanol were dried in a critical point dryer BIO-RAD (C.P.D750) and then coated with gold. Images were taken on a Jeol 5410 LV scanning electron microscope at 25 kV.

### References:

Naldini, L., Blomer, U., Gage, F. H., Trono, D., and Verma, I. M. (1996a). Efficient transfer, integration, and sustained long-term expression of the transgene in adult rat brains injected with a lentiviral vector. Proc Natl Acad Sci USA 93, 11382-11388.
Naldini, L., Blomer, U., Gallay, P., Ory, D., Mulligan, R., Gage, F. H., Verma, I. M., and Trono, D. (1996b). In vivo gene delivery and stable transduction of nondividing cells by a lentiviral vector. Science 272, 263-267.
   4 . PEM-3-Like is Required for HIV-1 Infectivity, and PEM-3-Like is an E3

PEM-3-like is required for HIV-1 infectivity

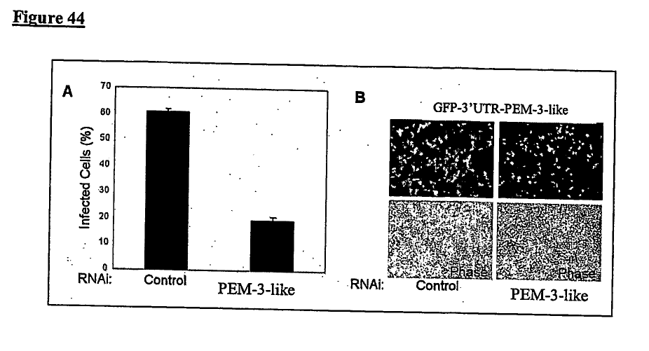

The production of infectious virus over a single cycle of HIV-1 replication, in the presence of normal or reduced levels of PEM-3-like was compared (FIG. 44A). To this end, cells were initially transfected with either a control or PEM-3-like specific siRNA (225) and then co-transfected with three plasmids encoding HIV-1 gag-pol, HIV-LTR-GFP and VSV-G-. Hence, the virus-producing cells release pseudotyped virions that contain VSV-G but do not by themselves encode an envelope protein and therefore, can infect target cells only once. Viruses were collected twenty-four hours post-transfection and used to infect HEK-293T cells. Infected target cells are detected by FACS analysis of GFP-positive cells. PEM-3-like reduction resulted in 60% reduction of virus infectivity (FIG. 44A), which correlated with the reduction in PEM-3-like levels as detected in parallel cultures co-transfected with RNAi and GFP-PEM-3-like tester plasmid (FIG. 44B), indicating that PEM-3-like is important for HIV-1 release.

PEM-3 -Like is a Ubiquitin Protein E3 Ligase

The presence of a RING finger domain in PEM-3-like suggested that it might be a ubiquitin protein ligase (E3) (Pickart, 2001). Three enzymes carry out covalent attachment of ubiquitin to target proteins: the ubiquitin-activating enzyme, El; a ubiquitin-conjugating enzyme, E2; and an E3. The E3 serves two roles: it specifically recognizes ubiquitination substrates and simultaneously recruits an E2. Ligation of ubiquitin is initiated by the formation of an isopeptide bond between the carboxyl terminus of ubiquitin and an s-amino group of a lysine residue on the target protein. Additional ubiquitin molecules can then be ligated to the initial ubiquitin molecule to form a poly-ubiquitinated protein (Hershko and Ciechanover, 1998). In the absence of an external substrate, E3's can catalyze self-ubiquitination, that is, transfer activated ubiquitin to a lysine side chain in the E3 polypeptide itself. Similar to trans-ubiquitination, self-ubiquitination is also dependent on the action of E1 and an E2 (Lorick et al., 1999).

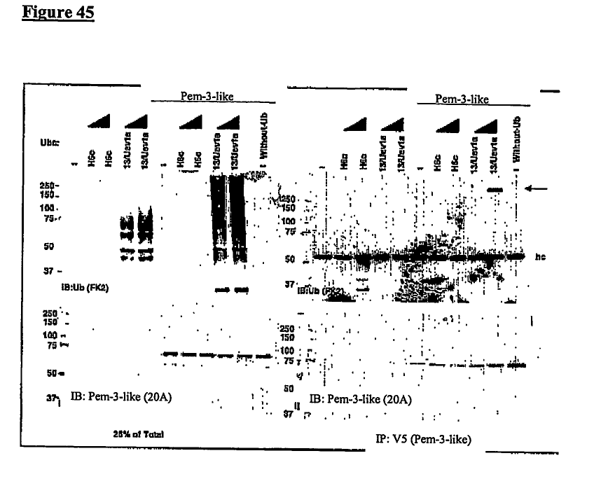

When a bacterially expressed glutathione-S-transferase protein (GST)-PEM-3-like fusion protein was incubated in vitro with E1, UBC13/Uev1 (E2), ubiquitin and ATP, high molecular weight PEM-3-like-ubiquitin adducts were detected by anti-ubiquitin immunoblot analysis (FIG. 45, right upper panel). In addition, free polyubiquitin chains were only generated in the presence of UBC13/Uev1 heterodimer and a complete ubiquitin conjugation system (FIG. 45, left upper panel).

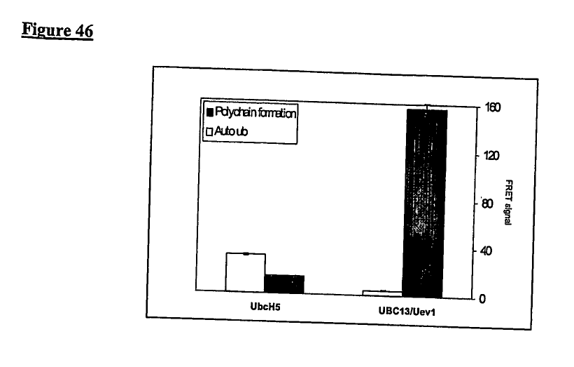

Analysis of PEM-3-like ubiquitin ligase was assessed also by FET analysis (FIG. 46). The results indicate that PEM-3-like acts as an E3 ligase with both UbcH5 and UBC13/Uev1.

Materials and Methods

Cell Culture and Transfections

Hela SS6 cells were grown in Dulbecco's modified Eagle's medium (DMEM) supplemented with 10% heat-inactivated fetal calf serum and 100 units/ml penicillin and 100 μg/ml streptomycin. For transfections, HeLa SS6 cells were grown to 50% confluency in DMEM containing 10% FCS without antibiotics. Cells were then transfected with the relevant double-stranded siRNA (50-100 nM) using lipofectamin 2000 (Invitrogen, Paisley, UK). On the day following the initial transfection, cells were split 1:3 in complete medium and co-transfected 24 hours later with HIV-1_{NLenv1} (2 μg per 6-well) (Schubert et al., 1995) and a second portion of double-stranded siRNA.

Infectivity Assay

HeLa SS6 cells were grown to 50% confluency in DMEM containing 10% FCS without antibiotics. Cells were then transfected (in duplicates) with the relevant double-stranded siRNA (50-100 nM) using lipofectamin 2000 (Invitrogen, Paisley, UK). On the day following the initial transfection, cells were co-transfected with pCMVΔ8.2 (Naldini et al., 1996a), encoding HIV-1 gag-pol (5 μg), pHR′-CMV-GFP (4 μg) (Naldini et al., 1996b), pMD.G (Naldini et al., 1996a), encoding VSV-G (1.5 μg) and a second portion of double-stranded siRNA (20 nM). Infection was performed twenty-four hours post-transfection, as follows: medium was collected from HeLa SS6 cells, polybrene was added to a final concentration of 8 μg/ml and the medium was palced on HEK-293T cells. Seventy-two hours post-infection cells were collected by trypsinization. Cells were fixed with 4% paraformaldehyde and analyzed for GFP-expression by FACS analysis.

In Vitro Ubiquitination Assays

Purified recombinant E1 (100 ng), UbcH5c or UbcH6c (E2) (250 ng), GST-PEM-3-like (400 ng) were incubated for 30 minutes at 37° C. in final volume of 20 μl containing 50 mM Hepes-NaOH pH 7.5, 1 mM DTT, 2 mM ATP, 5 mM MgCl₂, and 2.5 μg ubiquitin and 0.5 mg ubiquitin-biotin. Ubiqutinated PEM-3-like was separated by incubation with GSH-agarose. Both total and purified samples were were resolved on a 10% SDS gel and subjected to western blot analysis with anti-ubiquitin (Covance Research Products, Inc.) or anti-PEM-3-like antibodies.

For FRET analysis, self-ubiquitnation was determined by homogenous time-resolved fluorescence resonance energy transfer assay (TR-FRET). The conjugation of ubiquitin cryptate to GST-PEM-3-like and the binding of anti-GST tagged XL665 bring the two fluorophores into close proximity, which allows the FRET reaction to occur. To measure GST-PEM-3-like ubiquitination activity, GST-PEM-3-like (3ng) was incubated in reaction buffer (40 mM Hepes-NaOH, pH 7.5, 1 mM DTT, 2 mM ATP, 5 mM MgCl₂), with recombinant E1 (4 ng), UbcHSc or UBC13/Uev1 (10 ng), ubiquitin (1 ng) and ubiquitin-cryptate (2 ng) (CIS bio International) for 30 minutes at 37° C. Reactions were stopped with 0.5M EDTA. Anti-GST-XL₆₆₅ (CIS bio International) (50 nM) was then added to the reaction mixture for further 45 minutes incubation at room temperature. Emission at 620 nm and 665 nm was obtained after excitation at 380 nm in a fluorescence reader (RUBYstar, BMG Labtechnologies). The generation of PEM-3-like-ubiquitin-cryptate adducts was then determined by calculating the fluorescence resonance energy transfer (FRET, ΔF) using the following formula:
ΔF=[(S₆₆₅/S₆₂₀−B₆₆₅/B₆₂₀)/(B₆₆₅/B₆₂₀)]*100

S=actual fluorescence

B=Fluorescence obtained in parallel incubation without PEM-3-like.

Poly-ubiquitin chain formation was determined by homogenous time-resolved fluorescence resonance energy transfer assay (TR-FRET). The conjugation of polyubiquitin chains, formed by ubiquitin-cryptate and ubiquitin-biotin, was identified by streptavidin tagged XL665, which brings the two fluorophores into close proximity, and allows the FRET reaction to occur. All other reaction conditions were as detailed above.

5. Construction of PEM-3-Like Plasmids

A mammalian expression plasmid containing the C-terminal 464 amino acids of PEM-3-like identical to the protein translated from the mRNA AK096190..[gi:21755617] (CDS 188..1582). was constructed by joining together two I.M.A.G.E. clones (IMAGE:2748036 and IMAGE:5272241) into pcDNA3.1V5-His A (Invitrogen). The primers (518) CCGGGGATCCGGCATGATGGCGGCGATGCTGTCCCACG CCTACGGCCCCGGCGGTTGTGGGGCGGCGGCAGCCGCCCTGAACGGGGA and (516) GGTGTGGGTCTGCTGCTGAA were used to amplify clone IMAGE:2748036 and primers (394) CCATGATTCGTGCATCTCG and (515) CCGGTCTAGACTCGAGAGAGTGAATTTGGATTGCCTG were used to amplify clone IMAGE:5272241. The two overlapping PCR products were amplified with primers (514) CCGGGGATCCGAAATGATGGCGGCGATGCTGTC and 515 to create one 1421 bp product which was digested with BamHI and XhoI and ligated into pcDNA-V5-HisA (invitrogen) digested with same restriction enzymes to obtain pcDNA- PEM-3-like-V5-His in which the 464 aa protein is in frame with V-5-His tag from the vector. The plasmid was sequenced to verify that no mutations were introduced during the cloning procedure.

A bacterial expression plasmid was constructed by isolating a 1.5 kb BamHI-PmeI fragment from pcDNA- PEM-3-like-V5-His containing the 464 aa protein followed with the V5-His tag and ligating it into pGEX-6P-2 (Amersham Biosciences) digested with BamHI and SmaI to create pGEX-PEM-3-like-V5-His that codes for a fusion protein of PEM-3-like with GST (Glutathione-S-transferase) at the N-terminus and V5-His at the C-terminus. This plasmid was induced in BL21 E. coli cells by addition of 1 mM IPTG for 16 h at 16° C. The cells were lysed and the protein purified by glutathione sepharose chromatography.

6. Immunoprecipitation and Immunoblot of PEM-3-Like Protein

Materials and Methods

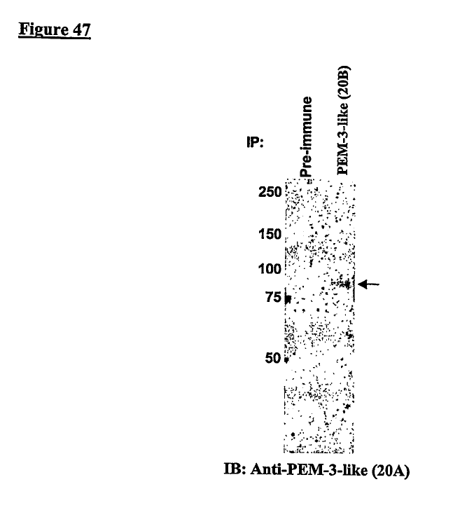

HeLa-SS6 cells from two 10 cm plates were washed three times with phosphate-buffered saline (PBS) and then solubilized by incubation on ice for 15 minutes in lysis buffer, 50 mM HEPES-NaOH, (pH 7.5), 150 mM NaCl, 1.5 mM MgC₂, 0.5% NP-40, 0.5% sodium deoxycholate, 1 mM EDTA, 1 mM EGTA and 1:100 dilution of protease inhibitor cocktail (Sigma.). The cell detergent extract was then centrifuged for 15 minutes at 14,000× g at 4° C. and subjected to immunoprecipitation with pre-immune or anti-PEM-3-like antibodies (20B, directed to the RING domain) cross-linked with DSS to Protein A-Sepharose beads (Amersham Biosciences, Corp.) using Seize X immunoprecipitation kit (Pierce). Beads were washed twice with high-salt buffer, once with medium-salt buffer and once with low-salt buffer. Bound proteins were resolved on a liner 8.5-12% gradient SDS-polyacrylamide gel, then transferred onto nitrocellulose membrane and subjected to immunoblot analysis with rabbit anti-PEM-3-like antibodies (20A, directed to the RING domain). The PEM-3-like protein was detected with a secondary Protein-A conjugated to horseradish peroxidase and detected by Enhanced Chemi-Luminescence (ECL) (Amersham Biosciences, Corp). (See FIG. 47).

7. Exemplary PEM-3-Like siRNA Target Sequences

siRNAs for PEM-3-like

| | | | |
|---|---|---|---|
| Number | Target sequence | siRNA sense strand | siRNA complementary strand |

| | | | |
|---|---|---|---|
| 225 | AACCACCGTCCAAGTCAGGGT | CCACCGUCCAAGUCAGGGUdTdT | ACCCUGACUUGGACGGUGGdTdT |
| | | (SEQ ID NO: 28) | (SEQ ID NO: 29) |
| 393 | AATGATAGTTCCAGTTCTCTA | UGAUAGUUCCAGUUCUCUAdTdT | UAGAGAACUGGAACUAUCAdTdT |
| | | (SEQ ID NO: 30) | (SEQ ID NO: 31) |
| 395 | GATAGTTCCAGTTCTCTAGGA | UAGUUCCAGUUCUCUAGGAdTdT | UCCUAGAGAACUGGAACUAdTdT |
| | | (SEQ ID NO: 32) | (SEQ ID NO: 33) |
| 397 | TAGGAAGTGGCTCTACAGATT | GGAAGUGGCUCUACAGAUUdTdT | AAUCUGUAGAGCCACUUCCdTdT |
| | | (SEQ ID NO: 34) | (SEQ ID NO: 35) |
| 399 | AAGTGGCTCTACAGATTCCTA | GUGGCUCUACAGAUUCCUAdTdT | UAGGAAUCUGUAGAGCCACdTdT |
| | | (SEQ ID NO: 36) | (SEQ ID NO: 37) |
| 401 | GACTTTAGTCCAACAAGCCCA | CUUUAGUCCAACAAGCCCAdTdT | UGGGCUUGUUGGACUAAAGdTdT |
| | | (SEQ ID NO: 38) | (SEQ ID NO: 39) |
| 403 | TAGTCCAACAAGCCCATTTAG | GUCCAACAAGCCCAUUUAGdTdT | CUAAAUGGGCUUGUUGGACdTdT |
| | | (SEQ ID NO: 40) | (SEQ ID NO: 41) |
| 405 | CAAGCCCATTTAGCACAGGAA | AGCCCAUUUAGCACAGGAAdTdT | UUCCUGUGCUAAAUGGGCUdTdT |
| | | (SEQ ID NO: 42) | (SEQ ID NO: 43) |
| 407 | AAGCCCATTTAGCACAGGAAA | GCCCAUUUAGCACAGGAAAdTdT | UUUCCUGUGCUAAAUGGGCdTdT |
| | | (SEQ ID NO: 44) | (SEQ ID NO: 45) |
| 409 | GAACCAGTTAACCCACTCTCT | ACCAGUUAACCCACUCUCUdTdT | AGAGAGUGGGUUAACUGGUdTdT |
| | | (SEQ ID NO: 46) | (SEQ ID NO: 47) |
| 411 | AACCATGTTGGCCTTCCAATA | CCAUGUUGGCCUUCCAAUAdTdT | UAUUGGAAGGCCAACAUGGdTdT |
| | | (SEQ ID NO: 48) | (SEQ ID NO: 49) |

8. PEM-3-Like/Nedd8 Fusion Protein Construction

In certain embodiments, the application relates to PEM-3-like polypeptides that are involved in neddylation, including PEM-3-like polypeptides that are neddylated. Neddylation of PEM-3-like polypeptides can be carried out as described in Amir, R E et al (2002) J Biol Chem 277:23253-23259.

Furthermore, a variety of PEM-3-like/Nedd8 fusion proteins can be created. One type of fusion protein is such that the Nedd8 sequence (underlined in the Examples below) is added at the C-terminus of PEM-3-like, either a full-length PEM-3-like (Example 1 below) or a partial region of PEM-3-like can be used (Example 2 below). A second type is such that the Nedd8 sequence is added at the N-terminus of PEM-3-like, this can be the natural N-terminus (Example 3 below) or the N-terminus of a partial region of PEM-3-like (Example 4 below). Construction of such fusion proteins can be performed by a variety of sub-cloning techniques known to one skilled in the art, such as overlaping PCR that was used to construct the plasmid pcDNA-PEM-3-like-V5-His, described above, from two separate clones.

Example 1 (SEQ ID NO: 50):
MPSGSSAALALAAAPAPLPQPPPPPPPPPPPLPPPSGGPELEGDGLLLRE
RLAALGLDDPSPAEPGAPALRAPAAAAQGQARRAAELSPEERAPPGRPGA
PEAAELELEEDEEEGEEAELDGDLLEEEELEEAEEEDRSSLLLLSPPAAT
ASQTQQIPGGSLGSVLLPAARFDAREAAAAAGVLYGGDDAQGMMAAMLSH
AYGPGGCGAAAAALNGEQAALLRRKSVNTTECVPVPSSEHVAEIVGRQGC
KIKALRAKTNTYIKTPVRGEEPIFVVTGRKEDVAMAKREILSAAEHFSMI
RASRNKNGPALGGLSCSPNLPGQTTVQVRVPYRVVGLVVGPKGATIKRIQ
QQTHTYIVTPSRDKEPVFEVTGMPENVDRAREEIEMHIAMRTGNYIELNE
ENDFHYNGTDVSFEGGTLGSAWLSSNPVPPSRARMISNYRNDSSSSLGSG
STDSYFGSNRLADFSPTSPFSTGNFWFGDTLPSVGSEDLAVDSPAFDSLP
TSAQTIWTPFEPVNPLSGFGSDPSGNMKTQRRGSQPSTPRLSPTFPESIE
HPLARRVRSDPPSTGNHVGLPIYIPAFSNGTNSYSSSNGGSTSSSPPESR
RKHDCVICFENEVIAALVPCGHNLFCMECANKICEKRTPSCPVCQTAVTQ
AIQIHSMLIKVKTLTGKEIEIDIEPTDKVERIKERVEEKEGIPPQQQRLI
YSGKQMNDEKTAADYKILGGSVLHLVLALRGGGGLRQ
Example 2 (SEQ ID NO: 51):
MMAAMLSHAYGPGGCGAAAAALNGEQAALLRRKSVNTTECVPVPSSEHVA
EIVGRQGCKIKALRAKTNTYIKTPVRGEEPIFVVTGRKEDVAMAKREILS
AAEHFSMIRASRNKNGPALGGLSCSPNLPGQTTVQVRVPYRVVGLVVGPK
GATIKRIQQQTHTYIVTPSRDKEPVFEVTGMPENVDRAREEIEMHIAMRT
GNYIELNEENDFHYNGTDVSFEGGTLGSAWLSSNPVPPSRARMISNYRND
SSSSLGSGSTDSYFGSNRLADFSPTSPFSTGNFWFGDTLPSVGSEDLAVD
SPAFDSLPTSAQTIWTPFEPVNPLSGFGSDPSGNMKTQRRGSQPSTPRLS
PTFPESIEHPLARRVRSDPPSTGNHVGLPIYIPAFSNGTNSYSSSNGGST
SSSPPESRRKHDCVICFENEVIAALVPCGHNLFCMECANKICEKRTPSCP
VCQTAVTQAIQIHSMLIKVKTLTGKIEIEIDIEPTDKVERIKERVEEKEG
IPPQQQRLIYSGKQMNDEKTAADYKILGGSVLHLVLALRGGGGLRQ
Example 3 (SEQ ID NO: 52):
MLIKVKTLTGKEIEIDIEPTDKVERIKERVEEKEGIPPQQQRLIYSGKQM
NDEKTAADYKILGGSVLHLVLALRGGGGLRQMPSGSSAALALAAAPAPLP
QPPPPPPPPPPPLPPPSGGPELEGDGLLLRERLAALGLDDPSPAEPGAPA
LRAPAAAAQGQARRAAELSPEERAPPGRPGAPEAAELELEEDEEEGEEAE
LDGDLLEEEELEEAEEEDRSSLLLLSPPAATASQTQQIPGGSLGSVLLPA
ARFDAREAAAAAGVLYGGDDAQGMMAAMLSHAYGPGGCGAAAAALNGEQA
ALLRRKSVNTTECVPVPSSEHVAEIVGRQGCKIKALRAKTNTYIKTPVRG
EEPIFVVTGRKEDVAMAKRRILSAARHFSMIRASRNKNGPALGGLSCSPN
LPGQTTVQVRVPYRVVGLVVGPKGATIKRIQQQTHTYIVTPSRDKEPVFE
VTGMPENVDRAREEIEMHIAMRTGNYIELNEENDFHYNGTDVSFEGGTLG
SAWLSSNPVPPSRARMISNYRNDSSSSLGSGSTDSYFGSNRLADFSPTSP
FSTGNFWFGDTLPSVGSEDLAVDSPAFDSLPTSAQTIWTPFEPVNPLSGF
GSDPSGNMKTQRRGSQPSTPRLSPTFPESIEHPLARRVRSDPPSTGNHVG
LPIYIPAFSNGTNSYSSSNGGSTSSSPPESRRKHDCVICFENEVIAALVP
CGHNLFCMECANKICEKRTPSCPVCQTAVTQAIQIHS
Example 4 (SEQ ID NO: 53):
MVKILTGKTLTGKEIEIDIEPTDKVERIKERVEEKEGIPPQQQRLIYSGK
QMNDEKTAADYKILGGSVLHLVLALRGGGGLRQMMAAMLSHAYGPGGCGA
AAAALNGEQAALLRRKSVNTTECVPVPSSEHVAEIVGRQGCKIKALRAKT
NTYIKTPVRGEEPIFVVTGRKEDVAMAKREILSAAEHFSMIRASRNKNGP
ALGGLSCSPNLPGQTTVQVRVPYRVVGLVVGPKGATIKRIQQQTHTYIVT
PSRDKEPVFEVTGMPENVDRAREEIEMHIAMRTGNYIELNEENDFHYNGT
DVSFEGGTLGSAWLSSNPVPPSRARMISNYRNDSSSSLGSGSTDSYFGSN
RLADFSPTSPFSTGNFWFGDTLPSVGSEDLAVDSPAFDSLPTSAQTIWTP
FEPVNPLSGFGSDPSGNMKTQRRGSQPSTPRLSPTFPESIEHPLARRVRS
DPPSTGNHVGLPIYIPAFSNGTNSYSSSNGGSTSSSPPESRRKHDCVICF
ENEVIAALVPCGHNLFCMECANKICEKRTPSCPVCQTAVTQAIQIHS

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

Equivalents

Those skilled in the art will recognize, or be able to ascertain using no more than routine experimentation, many equivalents to the specific embodiments of the invention described herein. Such equivalents are intended to be encompassed by the following claims.

1-7. (canceled)

8. A method for identifying an antiviral agent comprising:
(a) providing a PEM-3-like nucleic acid and a test agent; and
(b) identifying a test agent that binds to the PEM-3-like nucleic acid.

9. The method of claim 8, wherein the PEM-3-like nucleic acid is selected from the group consisting of SEQ ID NOS: 1, 3, 5, 7, 9, 11, 13, 15, 17, 19, 22, 24 and 25.

10. The method of claim 8, wherein the test agent is selected from the group consisting of: a ribonucleic acid, an antisense oligonucleotide, an RNAi construct, a DNA enzyme, and a ribozyme.

11. The method of claim 8, wherein binding of the test agent to said PEM-3-like nucleic acid decreases the level of a PEM-3-like transcript.

12. The method of claim 8, further comprising:
(a) administering a composition comprising the test agent to a cell transfected with at least a portion of a viral genome; and
(b) measuring the effect of the test agent on the production of viral or virus-like particles.

13. The method of claim 8, wherein the antiviral agent is effective against a virus selected from the group consisting of: an envelope virus, a retroid virus and a RNA virus.

14-40. (canceled)

41. A method for testing a ubiquitin-related activity of a PEM-3-like polypeptide comprising:
(a) forming a mixture compatible with the ubiquitin-related activity comprising: a ubiquitin; an El; an E2; and a PEM-3-like polypeptide; and
(b) detecting whether said ubiquitin binds to said PEM-3-like polypeptide.

42. The method of claim 41, wherein the PEM-3-like polypeptide is selected from the group consisting of SEQ ID NOS: 2, 4, 6, 8, 10, 12, 14, 16, 18, 20, 23, 26 and 27.

43-44. (canceled)

45. The method of claim 41, wherein the ubiquitin is detectably labeled.

46. The method of claim 41, wherein the PEM-3-like polypeptide is detectably labeled.

47. (canceled)

48. The method of claim 41, wherein the mixture further comprises NEDD8.

49. The method of claim 41, wherein the PEM-3-like polypeptide is neddylated.

50-68. (canceled)

69. The method of claim 10, wherein the RNAi construct is selected from the group consisting of: SEQ ID NOS: 28-49.

70-92. (canceled)

93. An isolated PEM-3-like nucleic acid comprising a nucleic acid sequence at least 85% identical to a nucleic acid sequence in selected from the group consisting of: SEQ ID NO: 22, SEQ ID NO: 24, and SEQ ID NO: 25.

94. The isolated PEM-3-like nucleic acid of claim 93, wherein the nucleic acid comprises the nucleic acid sequence depicted in SEQ ID NO: 22.

95. An isolated PEM-3-like polypeptide comprising an amino acid sequence encoded by a nucleic acid sequence according to claim 93, wherein the amino acid sequence comprises the amino acid sequence depicted in SEQ ID NO: 23.

96. (canceled)

97. The isolated PEM-3-like nucleic acid of claim 93, wherein the nucleic acid comprises the nucleic acid sequence depicted in SEQ ID NO: 24.

98. An isolated PEM-3-like polypeptide comprising an amino acid sequence encoded by a nucleic acid sequence according to claim 93, wherein the amino acid sequence comprises the amino acid sequence depicted in SEQ ID NO: 26.

99. (canceled)

100. The isolated PEM-3-like nucleic acid of claim 93, wherein the nucleic acid comprises the nucleic acid sequence depicted in SEQ ID NO: 25.

101. An isolated PEM-3-like polypeptide comprising an amino acid sequence encoded by a nucleic acid sequence according to claim 93, wherein the amino acid sequence comprises the amino acid sequence depicted in SEQ ID NO: 27.
